(19) United States
(12) Patent Application Publication
(10) Pub. No.: US 2017/0058026 A1
(43) Pub. Date: Mar. 2, 2017

(54) METHODS FOR MODULATING IMMUNE RESPONSES DURING CHRONIC IMMUNE CONDITIONS BY TARGETING IL-27 INDUCED PATHWAYS

(71) Applicant: The Brigham and Women's Hospital, Inc., Boston, MA (US)

(72) Inventors: Vijay K. Kuchroo, Newton, MA (US); Chen Zhu, Brookline, MA (US); Ana Carrizosa Anderson, Brookline, MA (US)

(73) Assignee: The Brigham and Women's Hospital, Inc., Boston, MA (US)

(21) Appl. No.: 15/339,218

(22) Filed: Oct. 31, 2016

Related U.S. Application Data
(62) Division of application No. 14/437,354, filed on Apr. 21, 2015.
(60) Provisional application No. 61/720,463, filed on Oct. 31, 2012.

(51) Int. Cl. C07K 16/24; C12N 15/113

(52) U.S. Cl. CPC C07K 16/244; C12N 15/1136; C07K 2317/76; C12N 2310/16; C12N 2310/11; C12N 2310/14

(57) ABSTRACT

Described herein are novel compositions comprising IL-27 or NFIL-3 modulators (i e , inhibitors or activators), and methods using these agents for targeting cells, such as functionally exhausted or unresponsive immune cells, and modulating TIM-3 activity or expression. These compositions, methods, and uses are useful for the treatment of chronic immune conditions, such as persistent infections, cancer, and autoimmune diseases.

### CROSS-REFERENCE TO RELATED APPLICATIONS

This application is a divisional application of U.S. application Ser. No. 14/437,354, filed on Apr. 21, 2015, which is a 35 U.S.C. 371 National Stage Application of International Application No. PCT/US2013/067481 filed on Oct. 30, 2013, which designates the United States, and which claims benefit under 35 U.S.C. §119(e) of U.S. Provisional Patent Application Ser. No. 61/720,463 filed on Oct. 31, 2012, the contents of which are herein incorporated by reference in their entirety.

### GOVERNMENT SUPPORT

This invention was made with Government Support under Grant Nos. P01 AI073748 and R01 NS045937 awarded by the National Institutes of Health. The Government has certain rights in the invention.

### SEQUENCE LISTING

The instant application contains a Sequence Listing which has been submitted in ASCII format via EFS-Web and is hereby incorporated by reference in its entirety. Said ASCII copy, created on Oct. 31, 2016, is named 043214-074833-DIV_SL.txt and is 27,012 bytes in size.

### FIELD OF THE INVENTION

The invention relates to compositions, methods, and uses for targeting IL-27 and IL-27 induced signaling pathways in the treatment of chronic immune conditions.

### BACKGROUND

T cell exhaustion is manifested by the progressive loss of function of antigen-specific T cells during chronic viral infections and cancers. Typically, antigen-specific T cells first lose IL-2 production, robust proliferation, and CTL function. Then the cells gradually stop secreting TNF, IFN-γ, and are eventually depleted by apoptosis (1-3).

Inhibitory receptors have been shown to play key roles in the regulation of T cell exhaustion. PD-1 is the prototypic molecule whose inhibitory function is essential to the induction of T cell exhaustion during chronic LCMV infection in mice and during chronic HIV infection in humans (4-7), and PD-1 expression is regarded as a benchmark for exhausted T cells. Control of T cell exhaustion has been shown to exhibit a hierarchical pattern, with increased expression of other inhibitory receptors delineating T cells with more deeply exhausted phenotypes (8, 9).

### SUMMARY OF THE INVENTION

The compositions, methods, and uses described herein are based, in part, on the novel discovery that IL-27 is a potent inducer of TIM-3 expression, and that IL-27-mediated induction of TIM-3 plays a critical role in functionally suppressing IFNγ secreting T cells and inducing T cell exhaustion during chronic immune conditions. TIM-3 is an inhibitory receptor and sustained TIM-3 expression has been shown to directly result in exhausted/dysregulated phenotype of antigen-specific T cells during chronic viral infections and cancers. As demonstrated herein, in response to IL-27, transcription factors NFIL3 and T-bet synergistically activate TIM-3 expression. In addition, IL-27 signaling results in profound permissive chromatin remodeling of the TIM-3 locus, favoring TIM-3 transcription. Thus, IL-27 signaling suppresses Type I effector T cell function via induction of TIM-3 expression and other anti-inflammatory molecules, including IL-10. Further, as demonstrated herein, IL-27R deficient (WSX-1^{-/-}) mice exhibit significant resistance to tumor growth that is accompanied by a failure to generate TIM-3 + exhausted T cells. Accordingly, the data provided herein identify IL-27 as a critical inducer of TIM-3-mediated T cell exhaustion/dysfunction during chronic conditions, and indicate that this induction is mediated, in part, by transcription factor NFIL3 induction.

Accordingly, provided herein, in some aspects are methods and uses for decreasing T-cell exhaustion in a subject in need thereof, the methods comprising administering to a subject an effective amount of a pharmaceutical composition comprising an IL-27 inhibitor.

In some embodiments of these methods and all such methods described herein, the IL-27 inhibitor binds IL-27 and inhibits its binding to IL-27R.

In some embodiments of these methods and all such methods described herein, the IL-27 inhibitor reduces expression of IL-27, an IL-27 subunit, or IL-27Ra.

In some embodiments of these methods and all such methods described herein, the IL-27 inhibitor decreases IL-27 mediated transcription factor induction or activation. In some embodiments, the transcription factor is NFIL-3 (nuclear factor, interleukin-3 regulated).

In some embodiments of these methods and all such methods described herein, the IL-27 inhibitor decreases NFIL-3 binding to a sequence at the TIM-3 locus. In some embodiments, the sequence at the TIM-3 locus comprises a sequence selected from any one of SEQ ID NO: 46-SEQ ID NO: 70.

In some embodiments of these methods and all such methods described herein, the IL-27 inhibitor decreases histone acetylation at a sequence at the TIM-3 locus. In some embodiments, the sequence at the TIM-3 locus comprises a sequence selected from any one of SEQ ID NO: 46-SEQ ID NO: 70.

In some embodiments of these methods and all such methods described herein, the IL-27 inhibitor decreases TIM-3 mRNA or protein upregulation or expression.

In some embodiments of these methods and all such methods described herein, the IL-27 inhibitor is an anti-IL-27 antibody or antigen-binding fragment thereof, a small molecule IL-27 inhibitor, an RNA or DNA aptamer that binds or physically interacts with IL-27 or IL-27R, an IL-27 or IL-27 receptor structural analog, a soluble IL-27 receptor, an IL-27 specific antisense molecule, or an IL-27 specific siRNA molecule.

In some embodiments of these methods and all such methods described herein, the subject being administered the IL-27 inhibitor is diagnosed as having a cancer or tumor. In some embodiments of these methods and all such methods described herein, the methods further comprise administering the subject diagnosed as having a cancer or tumor an anti-cancer therapy or agent.

In some embodiments of these methods and all such methods described herein, the subject being administered the IL-27 inhibitor is diagnosed as having a persistent infection.

In some embodiments of these methods and all such methods described herein, the subject being administered the IL-27 inhibitor has a chronic immune condition that comprises a population of functionally exhausted T cells. In some embodiments of these methods, the population of functionally exhausted T cells comprises a CD4+ T cell population.

In some aspects, provided herein are methods for decreasing T-cell exhaustion in a subject in need thereof, the methods comprising administering to a subject an effective amount of a pharmaceutical composition comprising an NFIL-3 inhibitor.

In some embodiments of these methods and all such methods described herein, the NFIL-3 inhibitor binds NFIL-3 and inhibits its binding to a target DNA sequence.

In some embodiments of these methods and all such methods described herein, the NFIL-3 inhibitor reduces expression of NFIL-3.

In some embodiments of these methods and all such methods described herein, the NFIL-3 inhibitor decreases NFIL-3 binding to a sequence at the TIM-3 locus. In some embodiments, the sequence at the TIM-3 locus comprises a sequence selected from any one of SEQ ID NO: 46-SEQ ID NO: 70.

In some embodiments of these methods and all such methods described herein, the NFIL-3 inhibitor decreases histone acetylation at a sequence at the TIM-3 locus. In some embodiments, the sequence at the TIM-3 locus comprises a sequence selected from any one of SEQ ID NO: 46-SEQ ID NO: 70.

In some embodiments of these methods and all such methods described herein, the NFIL-3 inhibitor decreases TIM-3 mRNA or protein upregulation or expression.

In some embodiments of these methods and all such methods described herein, the NFIL-3 inhibitor is an anti-NFIL-3 antibody or antigen-binding fragment thereof, a small molecule NFIL-3 inhibitor, an RNA or DNA aptamer that binds or physically interacts with NFIL-3, an NFIL-3 structural analog, an NFIL-3 specific antisense molecule, or an NFIL-3 specific siRNA molecule.

In some embodiments of these methods and all such methods described herein, the subject being administered the NFIL-3 inhibitor is diagnosed as having a cancer or tumor. In some embodiments of these methods and all such methods described herein, the methods further comprise administering the subject diagnosed as having a cancer or tumor an anti-cancer therapy or agent.

In some embodiments of these methods and all such methods described herein, the subject being administered the NFIL-3 inhibitor is diagnosed as having a persistent infection.

In some embodiments of these methods and all such methods described herein, the subject being administered the NFIL-3 inhibitor has a chronic immune condition that comprises a population of functionally exhausted T cells. In some embodiments of these methods, the population of functionally exhausted T cells comprises a CD4+ T cell population.

Also provided herein, in some aspects, are methods for promoting T cell exhaustion in a subject in need thereof, the methods comprising administering to a subject an effective amount of a pharmaceutical composition comprising an IL-27 activator.

In some embodiments of these methods and all such methods described herein, the IL-27 activator binds IL-27 and enhances its binding to IL-27R.

In some embodiments of these methods and all such methods described herein, the IL-27 activator increases expression of IL-27, an IL-27 subunit, or IL-27Ra.

In some embodiments of these methods and all such methods described herein, the IL-27 activator increases IL-27 mediated transcription factor induction or activation. In some embodiments, the transcription factor is NFIL-3 (nuclear factor, interleukin-3 regulated).

In some embodiments of these methods and all such methods described herein, the IL-27 activator increases NFIL-3 binding to a sequence at the TIM-3 locus. In some embodiments, the sequence at the TIM-3 locus comprises a sequence selected from any one of SEQ ID NO: 46-SEQ ID NO: 70.

In some embodiments of these methods and all such methods described herein, the IL-27 activator increases histone acetylation at a sequence at the TIM-3 locus. In some embodiments, the sequence at the TIM-3 locus comprises a sequence selected from any one of SEQ ID NO: 46-SEQ ID NO: 70.

In some embodiments of these methods and all such methods described herein, the IL-27 activator increases TIM-3 mRNA or protein upregulation or expression.

In some embodiments of these methods and all such methods described herein, the IL-27 activator is an anti-IL-27 antibody or antigen-binding fragment thereof, a small molecule IL-27 activator, an RNA or DNA aptamer that binds or physically interacts with IL-27 or IL-27R, or an IL-27 structural analog.

In some embodiments of these methods and all such methods described herein, the subject being administered the IL-27 activator is diagnosed as having an autoimmune disorder.

In some embodiments of these methods and all such methods described herein, the subject being administered the IL-27 activator is diagnosed as having graft versus host disease or is a transplant recipient.

In some aspects, provided herein are methods for for promoting T cell exhaustion in a subject in need thereof, the methods comprising administering to a subject an effective amount of a pharmaceutical composition comprising an NFIL-3 activator.

In some embodiments of these methods and all such methods described herein, the NFIL-3 activator binds NFIL-3 and enhances its binding to a target DNA sequence.

In some embodiments of these methods and all such methods described herein, the NFIL-3 activator increases expression of NFIL-3.

In some embodiments of these methods and all such methods described herein, the NFIL-3 activator increases NFIL-3 binding to a sequence at the TIM-3 locus. In some embodiments, the sequence at the TIM-3 locus comprises a sequence selected from any one of SEQ ID NO: 46-SEQ ID NO: 70.

In some embodiments of these methods and all such methods described herein, the NFIL-3 activator increases histone acetylation at a sequence at the TIM-3 locus. In some embodiments, the sequence at the TIM-3 locus comprises a sequence selected from any one of SEQ ID NO: 46-SEQ ID NO: 70.

In some embodiments of these methods and all such methods described herein, the NFIL-3 activator increases TIM-3 mRNA or protein upregulation or expression.

In some embodiments of these methods and all such methods described herein, the NFIL-3 activator is an anti-NFIL-3 antibody or antigen-binding fragment thereof, a small molecule NFIL-3 activator, an RNA or DNA aptamer that binds or physically interacts with NFIL-3, or an NFIL-3 structural analog.

In some embodiments of these methods and all such methods described herein, the subject being administered the NFIL-3 activator is diagnosed as having an autoimmune disorder.

In some embodiments of these methods and all such methods described herein, the subject being administered the NFIL-3 activator is diagnosed as having graft versus host disease or is a transplant recipient.

Also provided herein, in some aspects, are pharmaceutical compositions comprising an IL-27 inhibitor for use in decreasing T-cell exhaustion.

In some embodiments of these uses and all such uses described herein, the IL-27 inhibitor binds IL-27 and inhibits its binding to IL-27R.

In some embodiments of these uses and all such uses described herein, the IL-27 inhibitor reduces expression of IL-27, an IL-27 subunit, or IL-27Ra.

In some embodiments of these uses and all such uses described herein, the IL-27 inhibitor decreases IL-27 mediated transcription factor induction or activation.

In some embodiments of these uses and all such uses described herein, the transcription factor is NFIL-3 (nuclear factor, interleukin-3 regulated).

In some embodiments of these uses and all such uses described herein, the IL-27 inhibitor decreases NFIL-3 binding to a sequence at the TIM-3 locus.

In some embodiments of these uses and all such uses described herein, the IL-27 inhibitor decreases histone acetylation at a sequence at the TIM-3 locus.

In some embodiments of these uses and all such uses described herein, the sequence at the TIM-3 locus comprises a sequence selected from any one of SEQ ID NO: 46-SEQ ID NO: 70.

In some embodiments of these uses and all such uses described herein, the IL-27 inhibitor decreases TIM-3 mRNA or protein upregulation or expression.

In some embodiments of these uses and all such uses described herein, the IL-27 inhibitor is an anti-IL-27 antibody or antigen-binding fragment thereof, a small molecule IL-27 inhibitor, an RNA or DNA aptamer that binds or physically interacts with IL-27 or IL-27R, an IL-27 or IL-27 receptor structural analog, a soluble IL-27 receptor, an IL-27 specific antisense molecule, or an IL-27 specific siRNA molecule.

In some aspects, provided herein are pharmaceutical compositions comprising an NFIL-3 inhibitor for use in decreasing T-cell exhaustion.

In some embodiments of these uses and all such uses described herein, the NFIL-3 inhibitor binds NFIL-3 and inhibits its binding to a target DNA sequence.

In some embodiments of these uses and all such uses described herein, the NFIL-3 inhibitor reduces expression of NFIL-3.

In some embodiments of these uses and all such uses described herein, the NFIL-3 inhibitor decreases NFIL-3 binding to a sequence at the TIM-3 locus

In some embodiments of these uses and all such uses described herein, the NFIL-3 inhibitor decreases histone acetylation at a sequence at the TIM-3 locus.

In some embodiments of these uses and all such uses described herein, the sequence at the TIM-3 locus comprises a sequence selected from any one of SEQ ID NO: 46-SEQ ID NO: 70.

In some embodiments of these uses and all such uses described herein, the NFIL-3 inhibitor decreases TIM-3 mRNA or protein upregulation or expression.

In some embodiments of these uses and all such uses described herein, the NFIL-3 inhibitor is an anti-NFIL-3 antibody or antigen-binding fragment thereof, a small molecule NFIL-3 inhibitor, an RNA or DNA aptamer that binds or physically interacts with NFIL-3, an NFIL-3 structural analog, an NFIL-3 specific antisense molecule, or an NFIL-3 specific siRNA molecule.

In some embodiments of these uses and all such uses described herein, the T-cell exhaustion is caused or mediated by a cancer or tumor.

In some embodiments of these uses and all such uses described herein, the T-cell exhaustion is caused or meditated by a persistent infection.

In some embodiments of these uses and all such uses described herein, the T-cell exhaustion is caused or mediated by a chronic immune condition that comprises a population of functionally exhausted T cells.

In some embodiments of these uses and all such uses described herein, the population of functionally exhausted T cells comprises a CD4+ T cell population.

Also provided herein in some aspects are pharmaceutical compositions comprising an IL-27 activator for use in promoting T cell exhaustion.

In some embodiments of these uses and all such uses described herein, the IL-27 activator binds IL-27 and enhances its binding to IL-27R.

In some embodiments of these uses and all such uses described herein, the IL-27 activator increases expression of IL-27, an IL-27 subunit, or IL-27Ra.

In some embodiments of these uses and all such uses described herein, the IL-27 activator increases IL-27 mediated transcription factor induction or activation.

In some embodiments of these uses and all such uses described herein, the transcription factor is NFIL-3 (nuclear factor, interleukin-3 regulated).

In some embodiments of these uses and all such uses described herein, IL-27 activator increases NFIL-3 binding to a sequence at the TIM-3 locus

In some embodiments of these uses and all such uses described herein, the IL-27 activator increases histone acetylation at a sequence at the TIM-3 locus.

In some embodiments of these uses and all such uses described herein, the sequence at the TIM-3 locus comprises a sequence selected from any one of SEQ ID NO: 46-SEQ ID NO: 70.

In some embodiments of these uses and all such uses described herein, the IL-27 activator increases TIM-3 mRNA or protein upregulation or expression.

In some embodiments of these uses and all such uses described herein, the IL-27 activator is an anti-IL-27 antibody or antigen-binding fragment thereof, a small molecule IL-27 activator, an RNA or DNA aptamer that binds or physically interacts with IL-27 or IL-27R, or an IL-27 structural analog.

In some aspects, provided herein are pharmaceutical compositions comprising an NFIL-3 activator for use in promoting T cell exhaustion.

In some embodiments of these uses and all such uses described herein, the NFIL-3 activator binds NFIL-3 and enhances its binding to a target DNA sequence.

In some embodiments of these uses and all such uses described herein, the NFIL-3 activator increases expression of NFIL-3.

In some embodiments of these uses and all such uses described herein, the NFIL-3 activator increases NFIL-3 binding to a sequence at the TIM-3 locus

In some embodiments of these uses and all such uses described herein, the NFIL-3 activator increases histone acetylation at a sequence at the TIM-3 locus.

In some embodiments of these uses and all such uses described herein, the sequence at the TIM-3 locus comprises a sequence selected from any one of SEQ ID NO: 46-SEQ ID NO: 70.

In some embodiments of these uses and all such uses described herein, the NFIL-3 activator increases TIM-3 mRNA or protein upregulation or expression.

In some embodiments of these uses and all such uses described herein, the NFIL-3 activator is an anti-NFIL-3 antibody or antigen-binding fragment thereof, a small molecule NFIL-3 activator, an RNA or DNA aptamer that binds or physically interacts with NFIL-3, or an NFIL-3 structural analog.

In some embodiments of these uses and all such uses described herein, the promotion of T cell exhaustion is for treating an autoimmune disorder.

In some embodiments of these uses and all such uses described herein, the promotion of T cell exhaustion is for treating graft versus host disease or a transplant recipient.

### BRIEF DESCRIPTION OF THE FIGURES

FIGS. 1A-1C demonstrate that IL-27 is the most potent cytokine to induce Tim-3 expression. FIG. 1A. Naïve CD4 T cells were activated with anti-CD3 and anti-CD28 antibodies in the presence of different cytokine stimulations. The cells were harvested for quantitative PCR analysis for the expression of Tim-3 (Havcr2) 72 hours after activation. Havcr2 expression was normalized to that of β-actin. FIGS. 1B-1C. Naive CD4+ T cells were activated with anti-CD3 and anti-CD28 antibodies under neutral (Th0) or Thl culture conditions with or without the presence of IL-27. The cells were harvested to analyze the transcription of Tim-3 (Havcr2) and IL-10 by quantitative PCR 72 hours after activation (1B). To analyze the protein expression of Tim-3 and IL-10, the cells were restimulated by anti-CD3 and anti-CD28 antibodies for 24 to 36 hours and were subjected to Tim-3 and IL-10 detection by flow cytometry (1C). Data are representative of at least 3 independent experiments with similar results (1A, 1B: mean±s.d).

FIGS. 2A-2D demonstrate that transcription factor NFIL3 is required for the expression of both Tim-3 and IL-10. FIG. 2A. Naïve CD4+ T cells from C57BL/6 mice were stimulated with IL-27 and IL-12 during TcR activation. Five days later, the cells were restimulated with anti-CD3 and anti-CD28 for 24 hours and were subjected to detection of Tim-3 and IL-10 expression; FIG. 2B and FIG. 2D. Naïve CD4+ T cells from C57BL/6 mice were activated with anti-CD3 and anti-CD28 antibodies under neutral (Th0) or Th1 culture conditions with or without the presence of IL-27. RNA was isolated at 12, 24, 48, and 72 hours after TcR activation for quantitative PCR to determine the expression kinetics of IL-10, Tim-3 (Havcr2), T-bet (Tbx21), and NFIL3. Data are representative of at least 3 independent experiments with similar results. FIG. 2C. Gene profile studies. Naive CD4+ T cells from C57BL/6 mice were stimulated with anti-CD3 and anti-CD28 antibodies in the presence of IL-27 for 60 hours. The cells were then subjected to gene profile analysis. The scatter plot represents comparative transcriptome analysis between cells under neutral culture condition and cells treated with IL-27.

FIGS. 3A-3D demonstrate that IL-27-mediated Tim-3 expression is dependent on the functional cooperation between T-bet and NFIL3. FIG. 3A Naive CD4⁺ T cells were transduced with retrovirus carrying NFIL3 cDNA (NFIL3), T-bet cDNA (T-bet), or both NFIL3 and T-bet. Retrovirus empty vector transduced T cells were used as controls (GFP for NFIL3, Thy1.1 for T-bet). Four days after transduction, the expression of Tim-3, IL-10 and PD-1 was determined by flow cytometry. FIGS. 3B-3C Naïve CD4⁺ T cells were activated by anti-CD3 and anti-CD28 under neutral (Th0) and Th1 culture condition with or without the presence of IL-27. Three days after TcR activation, the expression of T-bet and NFIL3 was determined by quantitative PCR (3B) and Western blot (3C). FIG. 3D. Naïve CD4⁻ T cells from wild type (WT) C56BL/6 mice and NFIL3^{-/-} mice were activated with anti-CD3 and anti-CD28 under neutral (Th0) and Th1 culture condition with or without the presence of IL-27. Five days after TcR activation, cells were restimulated with anti-CD3 and CD28 for 24 hours and subjected to detection of the expression of Tim-3 and IL-10 by flow cytometry. Data are representative of at least 3 independent experiments with similar results (3B: mean±s.d)

FIGS. 4A-4D demonstrate that two non-redundant pathways through STAT1/T-bet and STAT3/NFIL3 involve IL-27-induced Tim-3 and IL-10 expression. FIGS. 4A-4B. Naïve CD4+ T cells purified from STAT1−/− mice (STAT1−/−) (4A) or STAT3fl/fl×CD4-Cre mice (STAT3 cko) (4B) and their wild type littermates (WT for STAT1−/− and STAT3fl/fl for STAT3 cko) were activated with anti-CD3 and anti-CD28 antibodies under neutral condition, or under IL-12 or IL-27 stimulation. Five days after TcR activation, the cells were restimulated by anti-CD3 and anti-CD28 for 24 hours and were subjected to the detection of Tim-3 and IL-10 by flow cytometry. FIGS. 4C-4D. Naïve CD4+ T cells as described in 4A and 4B were stimulated with anti-CD3 and anti-CD28 antibodies for 72 hours and were subjected to real time PCR analysis. The expression of T-bet (Tbx21) and NFIL3 was normalized to the (3-actin signal.

FIGS. 5A-5F demonstrate that IL-27-induced permissive chromatin modification in the Tim-3 locus is both NFIL3 and T-bet dependent. FIG. 5A. Mouse Tim-3 locus and the location of PCR primers for ChIP-QPCR analysis. FIG. 5B. Naïve CD4+ T cells from B6 mice were activated by plate-bound anti-CD3 and anti-CD28 antibodies under Th0 or Th1+IL-27 culture condition. The cells were restimulated on day 5 for 24 to 36 hours and were subjected to ChIP-PCR for detection of H3Ac enrichment in the Tim-3 locus. FIGS. 5C-5D. In the similar experiments, NFIL3^{-/-} CD4+ T cells and T-bet−/− CD4+ T cells were used to analyze the impact caused by their absence on H3Ac enrichment to the Tim-3 locus. FIG. 5E. NFIL3−/− and WT CD4+ T cells were activated by anti-CD3 and anti-CD28 antibodies under Th1 culture condition in the presence of IL-27. Four days after T cell activation, the cells were subjected to ChIP-QPCR to analyze NFIL3 enrichment to the Tim-3 locus. FIG. 5F. Expression plasmids for NFIL3 and T-bet were transiently transfected into 293T cells. Whole cell lysates were harvested 48 hours after transfection for two-way co-IP to detect the interaction between NFIL3 and T-bet. Data are representative of at least 2 independent experiments with similar results (FIGS. 5A-5F: mean±s.d).

FIGS. 6A-6F demonstrate that IL-27R deficient mice (WSX-1−/−) are resistant to tumor growth. FIG. 6A. B16F10 melanoma cells were implanted into C57BL/6 (WT) and WSX-1−/− mice and tumor growth was monitored. WSX-1−/− mice exhibited dramatically reduced tumor burden. FIG. 6B. Tumor-infiltrating lymphoctyes (TILs) from these mice were isolated and were subjected to real time PCR analysis for NFIL3 expression.FIG. 6C. The expression of Tim-3 and PD-1 on CD8+ TILs from WT and WSX-1−/− recipients tumor-bearing mice was determined by flow cytometry. FIG. 6D. The production of IL-2, IFN-γ and TNF in peripheral CD8+ T cells from WT and WSX-1−/− mice exhibited much higher production of IL-2, indicating that WSX-1−/− CD8+ T cells have more robust activation than wild type CD8+ T cells from control recipients. FIG. 6E. Total WT or NFIL3−/− T cells were transferred into Rag-1−/− recipients that were subsequently implanted with B16F10 melanoma. Tumor growth was compared between the recipients that received WT or NFIL3−/− T cells. FIG. 6F. In addition, TILs derived from WT and NFIL3−/− T cell-transferred recipients were examined for the expression of Tim-3 on PD-1+ TILs, and vice versa.

FIGS. 7A-7C demonsrate that ectopic expression of NFIL3 in CD4+ T cells attenuates the gut pathology in adoptive transferred colitis. FIG. 7A. Naïve CD4+ T cells from C57BL/6 mice were transduced with NFIL3-expressing retrovirus (NFIL3) or control empty retrovirus (GFP). Cells were i.p. injected into Rag1−/− recipient mice to induce gut inflammation. Wasting disease was monitored for 10 weeks after transfer. Statistics is based on the combination of total animals from two independent experiments. Data are shown as mean±SEM Mann Whiteny test two-tailed P=0.0064. FIG. 7B. Hematoxylin and eosin staining of small intestine tissue sections 10 weeks after adoptive transfer. FIG. 7C. The recipient mice were sacrificed 6 weeks after T cell transfer for ex vivo analysis of cytokine production and Tim-3 expression by flow cytometry.

FIG. 8 demonstrates that IL-27 is one of most potent cytokines to induce NFIL3 transcription. Naïve CD4+ T cells were activated by anti-CD3 and anti-CD28 antibodies in the presence of various cytokines for 48 hours. cDNA was prepared for real time PCR to quantify the expression of NFIL3 (mean±s.d). NFIL3 expression was normalized to the β-actin signal. Results represent at least 3 independent experiments.

FIGS. 9A-9B demonstrate that ectopic expression of NFIL3 results in cell death and induction of Tim-3 expression. FIG. 9A. Naïve CD4+ and CD8+ T cells were labeled with CellTrace™ Violet and were subjected to TcR activation by anti-CD3 and anti-CD28 antibodies. Shortly after activation, the cells were transduced with NFIL3 expressing retrovirus (NFIL3) or empty control retrovirus (GFP). Cell division was monitored by flow cytometry to detect the dilution of Violet signal. FIG. 9B. Naïve CD4+ T cells were activated with anti-CD3 and anti-CD28 antibodies and were subsequently transduced with NFIL3 expressing retrovirus (NFIL3) or empty control retrovirus (GFP). The expression of inhibitory receptors on transduced cells was examined by flow cytometry.

FIG. 10. Naïve CD4+ T cells were activated with anti-CD3 and anti-CD28 antibodies with or without the presence of IL-12 and IL-27 for 48 hours. Cells were then rested for 3 days post TCR activation, and were restimulated with anti-CD3 and anti-CD28 antibodies for 24 hours before subjected to ChIP-PCR analysis for Histone 3 Lysine 4 trimethylation (H3K3me) enrichment in the Tim-3 locus. A significant H3K3me enrichment was found in the Tim-3 promoter in the cells that were treated with IL-12 and IL-27.

FIGS. 11A-11B demonstrate that NFIL3 is important for Tim-3 expression in CD8⁺ T cells. FIG. 11A. Naïve CD8⁺ T cells were activated by anti-CD3 and anti-CD28 with or without the presence of IL-27. Three days after TcR activation, the expression of Tim-3 (Havcr2) was determined by quantitative PCR. FIG. 11B. To examine Tim-3 expression, cells were restimulated with anti-CD3 and CD28 for 24 hours and subjected to detection of the expression of Tim-3 by flow cytometry. Data are representative of at least 3 independent experiments with similar results (FIG. 11A: mean±s.d)

FIGS. 12A-12B show computational analysis of the human and mouse Tim-3 loci (Havcr2). FIG. 12A. Conserved non-coding sequence (CNS)s in the Tim-3 locus. By aligning the human and mouse Tim-3 locus in the ECR Browser (found on the worldwide web at dcode.org), 36 CNSs, having 70% or greater identity over at least 100 bp in length, were identified between human and mouse Tim-3 locus. CNSs that were determined to have potential NFIL3-binding sites were marked in bold. FIG. 12B. CNSs with significant NFIL3 enrichment that were identified by ChIP-QPCR. Numbers are the positions relative to the start of predicated CNS sequence identified by the ECR Browser. Bold sequences are putative NFIL3 binding sites.

FIGS. 13A-13B demonstrate that WSX-1^{-/-} mice are resistant to tumor growth. FIG. 13A. Lewis Lung carcinoma (LLC) cells were implanted into C57BL/6 (WT) and WSX-1^{-/-} mice and tumor growth was monitored in two dimensions. Statistics was based on combination of total animals from two independent experiments. FIG. 13B. The expression of Tim-3 and PD-1 on CD8⁺ TILs from WT and WSX-1^{-/-} recipient tumor-bearing mice was determined by flow cytometry. (13A and 13B: mean±SEM).

### DETAILED DESCRIPTION

Described herein are compositions, methods, and uses for modulating immune responses during chronic conditions by targeting IL-27, and IL-27-mediated induction of NFIL3 and TIM-3. These compositions, methods, and uses are based, in part, on the novel discovery that IL-27 is a potent inducer of TIM-3 expression, and that IL-27-mediated induction of TIM-3 has been shown to play a critical role in functionally suppressing IFNy secreting T cells and T cell exhaustion during chronic immune conditions. TIM-3 is an inhibitory receptor the expression of which on effector IFN-γ-producing T cells plays an important role in dampening T cell immunity. Sustained TIM-3 expression has been shown to directly result in exhausted/dysregulated phenotype of antigen-specific T cells during chronic viral infections and cancers. As shown herein, in response to IL-27, transcription factors NFIL3 and T-bet synergistically activate TIM-3 expression. In addition, IL-27 signaling results in profound permissive chromatin remodeling of the TIM-3 locus, favoring TIM-3 transcription. Thus, IL-27 signaling suppresses Type I effector T cell function via induction of TIM-3 expression and other anti-inflammatory molecules, including IL-10. Further, as demonstrated herein, IL-27R deficient (WSX-1−/−) mice exhibit significant resistance to tumor growth that is accompanied by a failure to generate TIM-3+ exhausted T cells. Accordingly, the data provided herein identify IL-27 as a critical inducer of TIM-3-mediated T cell exhaustion/dysfunction during chronic conditions, and indicate that this induction is mediated, in part, by transcription factor NFIL3 induction.

Accordingly, provided herein are compositions comprising IL-27 and NFIL3 modulators, such as agonists or activators and inhibitors or antagonists, and methods and uses thereof for modulating chronic immune conditions, such as cancer, infections, and autoimmune disorders, as described in more detail herein below.

### IL-27& IL-27 Signaling Pathways

IL-27 is a heterodimeric cytokine of the IL-6 and IL-12 family composed of the IL-27p28 and EBI3 subunits. IL-27p28 and EBI3 are produced primarily by antigen-presenting cells after stimulation by microbial products or inflammatory mediators. The IL-27 receptor is composed of WSX-1 (also known as T cell cytokine receptor), a type I cytokine receptor, and glycoprotein 130 (gp130), a receptor subunit utilized by several other IL-6 and IL-12 family members. Although gp130 expression is ubiquitous, WSX-1 expression is largely restricted to leukocytes, including T cells, natural killer (NK) cells, human monocytes, and human mast cells. IL-27 binds specifically to WSX-1, and EBI3 is required for signal transduction (E. D. Tait Wojno and C. A. Hunter, Trends Immunol. 2012 February; 33(2):91-7).

Accordingly, the term “ IL-27,” as used herein, refers to the heterodimer composed of: the mature form of the precursor IL-27p28 polypeptide having the amino acid sequence of: MGQTAGDLGWRLSLLLLPLLLVQAGVWGFPRPPGRPQLSLQELRREFTVSLHLARKLLSEVR GQAHRFAESHLPGVNLYLLPLGEQLPDVSLTFQAWRRLSDPERLCFISTTLQPFHALLGGLGT QGRWTNMERMQLWAMRLDLRDLQRHLRFQVLAAGFNLPEEEEEEEEEEEEERKGLLPGAL GSALQGPAQVSWPQLLSTYRLLHSLELVLSRAVRELLLLSKAGHSVWPLGFPTLSPQP (SEQ ID NO: 1), as described by, e.g., NP_663634.2, together with any naturally occurring allelic, splice variants, and processed forms (e.g., the mature form IL-27p28(29-243)) thereof, and the mature form of the precursor EBI3 or IL-27B polypeptide having the amino acid sequence of: MTPQLLLALVLWASCPPCSGRKGPPAALTLPRVQCRASRYPIAVDCSWTLPPAPNSTSPVSFI ATYRLGMAARGHSWPCLQQTPTSTSCTITDVQLFSMAPYVLNVTAVHPWGSSSSFVPFITEHI IKPDPPEGVRLSPLAERQLQVQWEPPGSWPFPEIFSLKYWIRYKRQGAARFHRVGPIEATSFIL RAVRPRARYYVQVAAQDLTDYGELSDWSLPATATMSLGK (SEQ ID NO: 2), as described by, e.g., NP_005746.2, together with any naturally occurring allelic, splice variants, and processed forms (e.g., the mature form IL-27B(21-229)) thereof. Typically, IL-27 refers to human IL-27. Specific residues of IL-27 can be referred to as, for example, “IL-27(62).”

IL-27 was initially described as a proinflammatory cytokine that promoted T helper (Th)1 responses. Subsequent studies in multiple models of infectious and autoimmune disease demonstrated an anti-inflammatory role for IL-27 in Th1, Th2 and Th17 responses, and recent work has shown that IL-27 can induce T cells to produce the anti-inflammatory cytokine IL-10. The consequences of IL-27 signaling appear to depend, in part, on the immunological context, the temporal regulation of IL-27 production, and tissue- and cell-specific expression of components of the IL-27 receptor (E.D. Tait Wojno and C.A. Hunter, Trends Immunol. 2012 February; 33(2):91-7).

IL-27 has been shown to promote the generation of Tr-1 cells that produce IL-10 by inducing expression of the activator protein-1 family transcription factor c-Maf. c-Maf directly transactivates the 1110 promoter to upregulate IL-10, and binds to the promoter of the common y chain cytokine Il21 to elicit IL-21 production that maintains IL-10 producers. Moreover, IL-27 signaling upregulates expression of the aryl hydrocarbon receptor (AhR), which partners with c-Maf to optimize interactions with the Il10 and Il21 promoters, further supporting Tr-1 development. IL-27-mediated IL-10 production also depends on STAT1 and STAT3 signaling, and the inducible co-stimulator (ICOS). IL-27 signaling is also believed to elicit Tfh responses by inducing c-Maf and IL-21 that promote Tfh activity. However, IL-27 alone does not cause CD4+ T cells to differentiate into functional Tfhs, and IL-27 signaling is not required for the generation of antibody responses in models of infection, allergy and autoimmunity. IL-27 also has direct effects on B cells. IL-27 has also been shown to regulate regulatory T cell (Treg) populations and acts as an antagonist of inducible Treg differentiation (E. D. Tait Wojno and C. A. Hunter, Trends Immunol. 2012 February; 33(2):91-7). Recently, it was also demonstrated that IL-27 priming of naïve CD4 and CD8 T cells upregulates expression of PD-L1 in a STAT1-dependent manner and such IL-27 primed cells can limit in trans the effect of pathogenic IL-17-producing Th17 cells in vitro and in vivo (Hirahara K. et al., Immunity. 2012 Jun. 29; 36(6):1017-30).

The results desecribed herein in detail below are in contrast to previous studies which describe that in the context of cancer, IL-27 therapy acts as a Treg inhibitor to enhance antitumor immunity in the suppressive tumor microenvironment. For example, in a murine model of neuroblastoma, in which IL-27 therapy inhibited IL-2-induced Treg expansion in the tumor, antitumor immune responses were promoted (R. Salcedo et al., J. Immunol., 182 (2009), pp. 4328-4338). In addition, IL-27 was shown to support directly the generation of potent antitumor CTLs and that IL-27 acts as a proinflammatory factor in this context to elicit IFN-γ production from CD8+ T cells in vivo in mice, and induce IFN-γ production and CTL activity in human CD8+ T cells (M. Hisada et al., Cancer Res., 64 (2004), pp. 1152-1156; R. Salcedo et al., J. Immunol., 173 (2004), pp. 7170-7182; Y. Cao et al., J. Immunol., 180 (2008), pp. 922-930; K. D. Mayer et al., J. Immunol., 180 (2008), pp. 693-697; and R. Schneider et al., Eur. J. Immunol., 41 (2011), pp. 47-59). Also, IL-27 has been reported to have direct antiproliferative effects on some tumors, including melanoma, lung carcinoma, and multiple myeloma (T. Yoshimoto et al., J. Immunol., 180 (2008), pp. 6527-6535; M. Y. Ho et al., J. Immunol., 183 (2009), pp. 6217-6226; and C. Cocco et al., Clin. Cancer Res., 16 (2010), pp. 4188-4197).

IL-27 Induction of NFIL3 and TIM3 Critical for T cell Exhaustion Phenotype

The results described herein demonstrate for the first time that IL-27 plays a critical role in the development of T cell exhaustion, in part by inducing the expression of the inhibitory molecule TIM-3 on T cells via the transcription factors NFIL-3 and T-bet. Further, as shown herein using IL-27 receptor deficient mice, in the absence of IL-27 signaling, tumor growth was suppressed and tumor burden controlled. In addition, ectopic expression of NFIL3 in T cells via retrovirus, and consequent increased expression of TIM-3, resulted in potent suppressive effects and induces exhaustion-like phenotypes in T cells, and reduced colitis severity, while NFIL3 deficiency in T cells resulted in reduced numbers of T cells with an exhausted phenotype. Accordingly, provided herein are novel compositions, methods, and uses to modulate chronic immune conditions by inhibiting or activating IL-27 mediated signaling pathways and downstream components thereof, such as NFIL-3, to modulate TIM-3 expression and/or activity and resulting suppression of immune response or development of T cell exhaustion phenotypes.

TIM-3 was originally identified as a mouse Th1-specific cell surface protein that was expressed after several rounds of in vitro Th1 differentiation, and was later shown to also be expressed on Th17 cells. In humans, TIM-3 is expressed on a subset of activated CD4+ T cells, on differentiated Thl cells, on some CD8+ T cells, and at lower levels on Th17 cells (Hastings W D, et al. 2009, Eur J Immunol. 39:2492-2501). TIM-3 is also expressed on cells of the innate immune system including mouse mast cells, subpopulations of macrophages and dendritic cells (DCs), NK and NKT cells, and human monocytes, and on murine primary bronchial epithelial cell lines. TIM-3 expression is regulated by the transcription factor T-bet. TIM-3 can generate an inhibitory signal resulting in apoptosis of Th1 and Tc1 cells, and can mediate phagocytosis of apoptotic cells and cross-presentation of antigen. Polymorphisms in TIM-1 and TIM-3 can reciprocally regulate the direction of T-cell responses (Freeman G J et al., Immunol Rev. 2010 Can; 235(1):172-89).

More recent studies have implicated TIM-3 in mediating T-cell dysfunction associated with chronic viral infections (Golden-Mason L, et al., 2009 J Virol; 83:9122-9130; Jones R B, et al., 2008 J Exp Med. 205:2763-2779). In progressive HIV infection, it was found that TIM-3 was expressed on about 50% of CD8+ T cells, and was expressed on virus-specific CD8+ T cells. It was found that blocking of the TIM-3 pathway ex vivo increased HIV-1-specific T cell responses. Notably, it was found that the TIM-3+ T cell subset was primarily distinct from the PD-1+ T cell subset (Golden-Mason L, et al., 2009 J Virol; 83:9122-9130).

In chronic HIV infection, TIM-3 expression was increased on CD4+ and CD8+ T cells, specifically HIV-specific CD8+ cytotoxic T cells (CTLs). It was found that a majority of virus-specific CTLs expressed PD-1, either alone, or co-expressed with TIM-3. Treatment with a blocking monoclonal antibody to TIM-3 reversed HIV-specific T cell exhaustion (Jones R B, et al., 2008 J Exp Med. 205:2763-2779).

Tumors express antigens that can be recognized by host T cells, but immunologic clearance of tumors is rare. Part of this failure is due to immune suppression by the tumor microenvironment. Recent work has indicated that a number of pathways, including, for example, the TIM-3 pathway, are involved in suppression of anti-cancer/tumor immune responses. Several studies have identified TIM-3 expression on exhausted T cells in both human cancer and in preclinical models of cancer. TIM-3 expression is specifically enriched on T cells present in tumor-infiltrated tissue and on tumor-infiltrating lymphocytes, relative to T cells either in peripheral lymphoid tissues or the blood of tumor-bearing hosts, indicating that TIM-3 is likely upregulated in response to tumor-derived environmental cues. Moreover, TIM-3 is often co-expressed with PD-1 and co-blockade of the TIM-3 and PD-1 signaling pathways has been shown to be more effective in restoring function to exhausted CD8+ T cells, and in controlling tumor growth than targeting either pathway alone. Co-blockade of TIM-3 and PD-1 has been shown to be effective in both prophylactic and therapeutic regimens against a wide variety of cancers (Anderson A C, Curr Opin Immunol. 2012 April; 24(2):213-6).

As used herein, an “immune response” refers to a response by a cell of the immune system, such as a B cell, T cell (CD4 or CD8), regulatory T cell, antigen-presenting cell, dendritic cell, monocyte, macrophage, NKT cell, NK cell, basophil, eosinophil, or neutrophil, to a stimulus. In some embodiments of the aspects described herein, the response is specific for a particular antigen (an “antigen-specific response”), and refers to a response by a CD4 T cell, CD8 T cell, or B cell via their antigen-specific receptor. In some embodiments of the aspects described herein, an immune response is a T cell response, such as a CD4+ response or a CD8+ response. Such responses by these cells can include, for example, cytotoxicity, proliferation, cytokine or chemokine production, trafficking, or phagocytosis, and can be dependent on the nature of the immune cell undergoing the response.

As used herein, the term “unresponsiveness” includes refractivity to activating receptor-mediated stimulation. Such refractivity is generally antigen-specific and persists after exposure to the antigen has ceased. Unresponsive immune cells can have a reduction of at least 10%, 20%, 30%, 40%, 50%, 60%, 70%, 80%, 90%, 95%, or even 100% in cytotoxic activity, cytokine production, proliferation, trafficking, phagocytotic activity, or any combination thereof, relative to a corresponding control immune cell of the same type.

As used herein, the terms “functional exhaustion” or “unresponsiveness” refers to a state of a cell where the cell does not perform its usual function or activity in response to normal input signals, and includes refractivity of immune cells to stimulation, such as stimulation via an activating receptor or a cytokine. Such a function or activity includes, but is not limited to, proliferation or cell division, entrance into the cell cycle, cytokine production, cytotoxicity, trafficking, phagocytotic activity, or any combination thereof. Normal input signals can include, but are not limited to, stimulation via a receptor (e.g., T cell receptor, B cell receptor, co-stimulatory receptor). Unresponsive immune cells can have a reduction of at least 10%, 20%, 30%, 40%, 50%, 60%, 70%, 80%, 90%, 95%, or even 100% in cytotoxic activity, cytokine production, proliferation, trafficking, phagocytotic activity, or any combination thereof, relative to a corresponding control immune cell of the same type. In some particular embodiments of the aspects described herein, a cell that is functionally exhausted is a CD4 or helper T lymphocyte that expresses the CD4 cell surface marker. Such CD4 cells normally proliferate, and/or produce cytokines, such as IL-2, TNFα, IFNγ, IL-4, IL-5, IL-17, or a combination thereof, in response to T cell receptor and/or co-stimulatory receptor stimulation. Thus, a functionally exhausted or unresponsive CD4 T cell is one which does not proliferate and/or produce cytokines, such as IL-2, TNFα, IFNγ, in response to normal input signals. The cytokines produced by CD4 T cells act, in part, to activate and/or otherwise modulate, i.e., “provide help,” to other immune cells such as B cells and CD8+ cells.

As used herein, the term “reduces T cell tolerance” means that a given treatment or set of conditions leads to reduced T cell tolerance, i.e., greater T cell activity, responsiveness, and/or ability or receptiveness with regards to activation. Methods of measuring T cell activity are known in the art. By way of non-limiting example, T cell tolerance can be induced by contacting T cells with recall antigen, anti-CD3 in the absence of costimulation, and/or ionomycin. Levels of, e.g. LDH-A, RAB10, and/or ZAP70 (both intracellular or secreted) can be monitored, for example, to determine the extent of T cell tolerogenesis (with levels of IL-2, interferon-y and TNF correlating with increased T cell tolerance). The response of cells pre-treated with, e.g. ionomycin, to an antigen can also be measured in order to determine the extent of T cell tolerance in a cell or population of cells, e.g. by monitoring the level of secreted and/or intracellular IL-2 and/or TNF-α (see, e.g. Macian et al. Cell 2002 109:719-731; which is incorporated by reference herein in its entirety). Other characteristics of T cells having undergone adaptive tolerance is that they have increased levels of Fyn and ZAP-70/Syk, Cbl-b, GRAIL, Ikaros, CREM (cAMP response element modulator), B lymphocyte-induced maturation protein-1 (Blimp-1), PD1, CD5, and SHP2; increased phosphorylation of ZAP-70/Syk, LAT, PLCγ1/2, ERK, PKC-Θ/IKBA; increased activation of intracellular calcium levels; decreased histone acetylation or hypoacetylation and/or increased CpG methylation at the IL-2 locus. Thus, in some embodiments, modulation of one or more of any of these parameters can be assayed to determine whether one or more IL-27 or NFIL-3 modulating agents modulates an immune response in vivo or modulates immune tolerance.

Modulation of T cell tolerance can also be measured by determining the proliferation of T cells in the presence of a relevant antigen assayed, e.g. by a ³H-thymidine incorporation assay or cell number. Markers of T cell activation after exposure to the relevant antigen can also be assayed, e.g. flow cytometry analysis of cell surface markers indicative of T cell activation (e.g. CD69, CD30, CD25, and HLA-DR). Reduced T cell activation in response to antigen-challenge is indicative of tolerance induction. Conversely, increased T cell activation in response to antigen-challenge is indicative of reduced tolerance.

Modulation of T cell tolerance can also be measured, in some embodiments, by determining the degree to which the modulating agent inhibits or increase the activity of its target. For example, the SEB model can be used to measure T cell tolerance and modulation thereof. In normal mice, neonatal injection of staphylococcal enterotoxin B (SEB) induces tolerance in T cells that express reactive T cell receptor (TCR) V beta regions. If, in the presence of an IL-27 or NFIL-3 modulating, T cells expressing reactive TCR V beta regions (e.g., Vbeta8) display a statistically significant reduction or increase in T cell activity than T cells not contacted with the modulating agent, the modulating agent is one that modulates T cell tolerance.

Other in vivo models of peripheral tolerance that can be used in some aspects and embodiments to measure modulation in T cell tolerance using the modulating agents described herein include, for example, models for peripheral tolerance in which homogeneous populations of T cells from TCR transgenic and double transgenic mice are transferred into hosts that constitutively express the antigen recognized by the transferred T cells, e.g., the H-Y antigen TCR transgenic; pigeon cytochrome C antigen TCR transgenic; or hemagglutinin (HA) TCR transgenic. In such models, T cells expressing the TCR specific for the antigen constitutively or inducibly expressed by the recipient mice typically undergo an immediate expansion and proliferative phase, followed by a period of unresponsiveness, which is reversed when the antigen is removed and/or antigen expression is inhibited. Accordingly, if, in the presence of an IL-27 or NFIL-3 inhibitory agent, for example, in such models if the T cells proliferate or expand, show cytokine activity, etc. significantly more than T cells in the absence of the inhibitory agent, than that agent is one that reduces T cell tolerance. Such measurements of proliferation can occur in vivo using T cells labeled with BrDU, CFSE or another intravital dye that allows tracking of proliferation prior to transferring to a recipient animal expressing the antigen, or cytokine reporter T cells, or using ex vivo methods to analyze cellular proliferation and/or cytokine production, such as thymidine proliferation assays, ELISA, cytokine bead assays, and the like.

Modulation of T cell tolerance can also be assessed by examination of tumor infiltrating lymphocytes or T lymphocytes within lymph nodes that drain from an established tumor. Such T cells exhibit features of “exhaustion” through expression of cell surface molecules, such asTIM-3, for example, and decreased secretion of cytokines such as interferon-y. Accordingly, if, in the presence of an inhibitory agent, increased quantities of T cells with, for example, 1) antigen specificity for tumor associated antigens are observed (e.g. as determined by major histocompatibility complex class I or class II tetramers which contain tumor associated peptides) and/or 2) that are capable of secreting high levels of interferon-y and cytolytic effector molecules such as granzyme-B, relative to that observed in the absence of the inhibitory agent, this would be evidence that T cell tolerance had been reduced.

TIM-3 is a Type I cell-surface glycoprotein that comprises an N-terminal immunoglobulin (Ig)-like domain, a mucin domain with 0-linked glycosylations and with N-linked glycosylations close to the membrane, a single transmembrane domain, and a cytoplasmic region with tyrosine phosphorylation motif(s). TIM-3 is a member of the T cell/transmembrane, immunoglobulin, and mucin (TIM) gene family. The term “TIM-3” as used herein, refers to the 301 amino acid polypeptide having the amino acid sequence of: MFSHLPFDCVLLLLLLLLTRSSEVEYRAEVGQNAYLPCFYTPAAPGNLVPVCWGKGACPVFE CGNVVLRTDERDVNYWTSRYWLNGDFRKGDVSLTIENVTLADSGIYCCRIQIPGIMNDEKFN LKLVIKPAKVTPAPTLQRDFTAAFPRMLTTRGHGPPAETQTLGSLPDINLTQISTLANELRDSR LANDLRDSGATIRIGIYIGAGICAGLALALIFGALIFKWYSHSKEKIQNLSLISLANLPPSGLAN AVAEGIRSEENIYTIEENVYEVEEPNEYYCYVSSRQQPSQPLGCRFAMP (SEQ ID NO: 3), as described by, e.g., AAL65157, together with any naturally occurring allelic, splice variants, and processed forms thereof. Typically, TIM-3 refers to human TIM-3. The term “TIM-3” is also used to refer to truncated forms or fragments of the TIM-3 polypeptide. Reference to any such forms or fragments of TIM-3 can be identified in the application, e.g., by “TIM-3 (24-131).” Specific residues of TIM-3 can be referred to as, for example, “TIM-3(62).”

TIM-3 has two known ligands, galectin-9 and phosphatidylserine. Galectin-9 is an S-type lectin with two distinct carbohydrate recognition domains joined by a long flexible linker, and has an enhanced affinity for larger poly-N-acetyllactosamine-containing structures. Galectin-9 does not have a signal sequence and is localized in the cytoplasm. However, it can be secreted and exerts its function by binding to glycoproteins on the target cell surface via their carbohydrate chains (Freeman G J et al., Immunol Rev. 2010 Can; 235(1):172-89).

Galectin-9 is expressed broadly including in immune cells and the epithelium of the gastrointestinal tract. Galectin-9 expression is particularly high in mast cells and also found in T cells, B cells, macrophages, endothelial cells, and fibroblasts. Galectin-9 production can be upregulated by IFN-γ. Galectin-9 has also been reported to exert various biologic functions via interaction with CD44 and IgE. Engagement of TIM-3 by galectin-9 leads toThl cell death and a consequent decline in IFN-γ production. When given in vivo, galectin-9 had beneficial effects in several murine disease models, including an EAE model, a mouse model of arthritis, in cardiac and skin allograft transplant models, and contact hypersensitivity and psoriatic models (Freeman G J et al., Immunol Rev. 2010 Can; 235(1):172-89). Residues important for TIM-3 binding to galectin-9 include TIM-3(44), TIM-3(74), and TIM-3(100), which undergo N- and/or O-glycosylation.

Both human and mouse TIM-3 have been shown to be receptors for phosphatidylserine (PtdSer), based on binding studies, mutagenesis, and a co-crystal structure, and it has been shown that TIM-3-expressing cells bound and/or engulfed apoptotic cells expressing PtdSer. Interaction of TIM-3 with PtdSer does not exclude an interaction with galectin-9 as the binding sites have been found to be on opposite sides of the IgV domain. Residues important for TIM-3 binding to PtdSer include TIM-3(50), TIM-3(62), TIM-3(69), TIM-3(112), and TIM-3(121).

Although the function of TIM-3 has been linked to the suppression of T cell immunity, and different ligands for TIM-3 have been identified, less is known in regard to its regulation and induction by different factors. The results described herein demonstrate for the first time that IL-27 regulates TIM-3, in part by inducing the expression of the transcription factors NFIL-3 and T-bet, resulting in expression of TIM-3 on T cells, thus providing novel upstream targets for modulating TIM-3-mediated T cell exhaustion and chronic immune conditions. Thus, provided herein, in different aspects, are modulators of IL-27 signaling, including inhibitor/antagonist agents and activator/agonist agents, and/or modulators of NFIL3 activity and/or function, including NFIL-3 inhibitor/antagonist agents and activator/agonist agents, for modulating T cell exhaustion phenotypes mediated by TIM-3, and methods thereof for modulating TIM-3 activity and expression and consequent T cell exhaustion phenotypes.

As used herein, in regard to the IL-27 inhibitor/antagonist agents and activator/agonist agents and the NFIL-3 inhibitor/antagonist agents and activator/agonist agents described herein, “modulating” or “to modulate” generally means either reducing or inhibiting the activity of, or alternatively increasing the activity of, a target or antigen, such as IL-27 or NFIL-3, as measured using a suitable in vitro, cellular or in vivo assay, such as those described herein in the Examples. In particular, “modulating” or “to modulate” can mean either reducing or inhibiting the activity of, or alternatively increasing a (relevant or intended) biological activity of, a target or antigen, as measured using a suitable in vitro, cellular or in vivo assay (which will usually depend on the target or antigen involved), by at least 5%, at least 10%, at least 25%, at least 50%, at least 60%, at least 70%, at least 80%, or 90% or more, compared to activity of the target or antigen in the same assay under the same conditions but without the presence of the inhibitor/antagonist agents or activator/agonist agents described herein.

As will be clear to the skilled person, “modulating” can also involve effecting a change (which can either be an increase or a decrease) in affinity, avidity, specificity and/or selectivity of a target or antigen for one or more of its ligands, binding partners, partners for association into a homomultimeric or heteromultimeric form, or substrates; and/or effecting a change (which can either be an increase or a decrease) in the sensitivity of the target or antigen for one or more conditions in the medium or surroundings in which the target or antigen is present (such as pH, ion strength, the presence of co-factors, etc.), compared to the same conditions but without the presence of a modulating agent. Again, this can be determined in any suitable manner and/or using any suitable assay known per se, depending on the target or antigen involved. In particular, an action as an inhibitor/antagonist or activator/agonist can be such that an intended biological or physiological activity is increased or decreased, respectively, by at least 5%, at least 10%, at least 25%, at least 50%, at least 60%, at least 70%, at least 80%, or 90% or more, compared to the biological or physiological activity in the same assay under the same conditions but without the presence of the inhibitor/antagonist agent or activator/agonist agent. Modulating can, for example, also involve allosteric modulation of the target or antigen; and/or reducing or inhibiting the binding of the target or antigen to one of its substrates or ligands and/or competing with a natural ligand, substrate for binding to the target or antigen. Modulating can also involve activating the target or antigen or the mechanism or pathway in which it is involved. Modulating can for example also involve effecting a change in respect of the folding or conformation of the target or antigen, or in respect of the ability of the target or antigen to fold, to change its conformation (for example, upon binding of a ligand), to associate with other (sub)units, or to disassociate. Such a change will have a functional effect.

Accordingly, in some aspects, provided herein, are compositions comprising IL-27 inhibitors or antagonists for use in decreasing T cell exhaustion by inhibiting TIM-3 induction and/or activity.

As used herein, the terms “IL-27 inhibitor,” “IL-27 antagonist,” “IL-27 inhibitor agent,” and “IL-27 antagonist agent” refer to a molecule or agent that significantly blocks, inhibits, reduces, or interferes with IL-27 (mammalian, such as human IL-27) biological activity in vitro, in situ, and/or in vivo, including activity of downstream pathways mediated by IL-27 signaling, such as, for example, transcription factor induction (e.g., NFIL3 or T-bet induction), IL-10 induction, histone acetylation at the TIM-3 locus, TIM-3 mRNA or protein upregulation, and/or elicitation of a cellular response to IL-27. Exemplary IL-27 inhibitors contemplated for use in the various aspects and embodiments described herein include, but are not limited to, anti-IL-27 antibodies or antigen-binding fragments thereof that specifically bind to IL-27 or one or both subunits of IL-27 (i.e., IL-27p28 and/or EBI3/IL27B); anti-sense molecules directed to a nucleic acid encoding either subunit of IL-27 (i.e., IL-27p28 and/or EBI3/IL27B); short interfering RNA (“siRNA”) molecules directed to a nucleic acid encoding one or both subunits of IL-27 (i.e., IL-27p28 or IL-27Ebi3); or IL-27Ra, an IL-27 inhibitory compound; RNA or DNA aptamers that bind to IL-27, one or both subunits of IL-27, or to IL-27Ra and inhibit/reduce/block IL-27 mediated signaling; IL-27 structural analogs; soluble IL-27Ra proteins or fusion polypeptides thereof; anti-IL-27Ra antibodies or antigen-binding fragments thereof; and small molecule agents that target or bind to IL-27, one or both subunits of IL-27, or to IL-27Ra. In some embodiments of these aspects and all such aspects described herein, an IL-27 inhibitor (e.g., an antibody or antigen-binding fragment thereof) binds (physically interacts with) IL-27, binds to an IL-27Ra, targets downstream IL-27Ra signaling, and/or inhibits (reduces) IL-27 synthesis, production or release. In some embodiments of these aspects and all such aspects described herein, an IL-27 inhibitor binds IL-27 and prevents its binding to its receptor. In some embodiments of these aspects and all such aspects described herein, an IL-27 inhibitor specifically reduces or eliminates expression (i.e., transcription or translation) of IL-27, an IL-27 subunit, or IL-27Ra.

In some embodiments of the compositions, methods, and uses described herein, the IL-27 inhibitor inhibits IL-27 mediated signal transduction. In some embodiments of the compositions, methods, and uses described herein, the IL-27 inhibitor targets IL-27 mediated transcription factor induction or activation, for example, NFIL3 or T-bet induction or activation. In some embodiments of the compositions, methods, and uses described herein, the IL-27 inhibitor interferes with NFIL-3 binding to conserved cis-regulatory regions or sequences at the TIM-3 locus, such as, for example, a sequence selected from any one of SEQ ID NO: 46-SEQ ID NO: 70.

In some embodiments of the compositions, methods, and uses described herein, the IL-27 inhibitor decreases or inhibits IL-27-mediated histone acetylation at a sequence at the TIM-3 locus, such as histone acetylation at intron 1. In some embodiments of the compositions and methods described herein, the IL-27 inhibitor targets IL-27-mediated TIM-3 mRNA or protein upregulation. In some embodiments of the compositions and methods described herein, the IL-27 inhibitor targets IL-27-induced IL-10 production.

In some embodiments of the compositions, methods, and uses described herein, the IL-27 inhibitor is an antibody or antigen-binding fragment thereof that selectively binds or physically interacts with a subunit of IL-27 (IL-27p28 or IL-27Ebi3). In some embodiments of the compositions, methods, and uses described herein, the IL-27 inhibitor is an antibody or antigen-binding fragment thereof that binds to IL-27p28 or IL-27Ebi3 and inhibits and/or blocks and/or prevents formation of the heterodimeric IL-27. In some embodiments of the compositions, methods, and uses described herein, the IL-27 inhibitor is an antibody or antigen-binding fragment thereof that binds to IL-27p28 and inhibits and/or blocks and/or prevents formation of the heterodimeric IL-27. In some embodiments of the compositions, methods, and uses described herein, the IL-27 inhibitor is an antibody or antigen-binding fragment thereof that binds to IL-27Ebi3 and inhibits and/or blocks and/or prevents formation of the heterodimeric IL-27.

In some embodiments of the compositions, methods, and uses described herein, the binding sites of the IL-27 inhibitors, such as an antibody or antigen-binding fragment thereof, are directed against an IL-27R ligand interaction site. In some embodiments of the compositions, methods, and uses described herein, the binding sites of the IL-27 inhibitors are directed against a site on a target in the proximity of the ligand interaction site, in order to provide steric hindrance for the interaction of the target (e.g., IL-27) with its receptor (e.g., IL-27Ra). By binding to an IL-27 ligand interaction site, an IL-27 inhibitor described herein can reduce or inhibit the activity or expression of IL-27, and downstream IL-27 signaling consequences (e.g., transcription factor induction (e.g., NFIL3 or T-bet induction), IL-10 induction, histone acetylation at a sequence at the TIM-3 locus, TIM-3 mRNA or protein upregulation, and/or elicitation of a cellular response to IL-27). In some embodiments of the compositions, methods, and uses described herein, the IL-27 inhibitor is an anti-sense molecule directed to a nucleic acid encoding either subunit of IL-27 (i.e., IL-27p28 and/or EBI3/IL27B). In some embodiments of the compositions, methods, and uses described herein, the IL-27 inhibitor is a short interfering RNA molecule directed to a nucleic acid encoding acid encoding one or both subunits of IL-27 (i.e., IL-27p28 or IL-27Ebi3); or IL-27Ra3. In some embodiments of the compositions, methods, and uses described herein, the IL-27 inhibitor is an RNA or DNA aptamer that binds to IL-27, one or both subunits of IL-27, or to IL-27Ra. In some embodiments of the compositions, methods, and uses described herein, the IL-27 inhibitor is a small molecule compound or agent that targets or binds to IL-27, one or both subunits of IL-27, or to IL-27Ra.

As used herein, an IL-27 inhibitor or antagonist has the ability to reduce the activity and/or expression of IL-27 in a cell (e.g., T cells, such as CD8+ or CD4+ T cells) by at least 5%, at least 10%, at least 20%, at least 30%, at least 40%, at least 50%, at least 60%, at least 70%, at least 80%, at least 90%, at least 95%, at least 98%, at least 99%, or more, relative to the activity or expression level in the absence of the IL-27 antagonist.

In some embodiments of the compositions, methods, and uses described herein, the IL-27 inhibitor is an antibody or antigen-binding fragment thereof that binds to the heterodimeric IL-27 but does not bind to either the IL27p28 polypeptide or IL-27Ebi3 polypeptide alone. In other words, in some embodiments of the compositions, methods, and uses described herein, the IL-27 inhibitor is an antibody or antigen-binding fragment thereof that binds to an epitope found in the heterodimeric IL-27 but not in the IL27p28 polypeptide or IL-27Ebi3 polypeptide alone. In some embodiments of the compositions, methods, and uses described herein, the IL-27 inhibitor is an antibody or antigen-binding fragment thereof that binds or physically interacts with heterodimeric IL-27, and blocks interactions between IL-27 and its receptor. In some embodiments of the compositions, methods, and uses described herein, the IL-27 inhibitor is an antibody or antigen-binding fragment thereof that binds to an epitope on the IL27p28 subunit of IL-27. In some embodiments of the compositions, methods, and uses described herein, the IL-27 inhibitor is an antibody or antigen-binding fragment thereof that binds to an epitope on the IL-27Ebi3 subunit of IL-27. In some embodiments of the compositions, methods, and uses described herein, the IL-27 inhibitor is an antibody or antigen-binding fragment thereof that binds to an epitope formed from both subunits of IL-27.

In some embodiments of the compositions, methods, and uses described herein, the IL-27 inhibitor is an antibody or antigen-binding fragment thereof that binds or physically interacts with IL-27Ra. In some embodiments of the compositions, methods, and uses described herein, the IL-27 inhibitor is an antibody or antigen-binding fragment thereof that binds IL-27Ra and inhibits and/or prevents formation of heterodimeric IL-27 receptor. In some embodiments of the compositions, methods, and uses described herein, the IL-27 inhibitor is an antibody or antigen-binding fragment thereof that binds IL-27Ra and inhibits and/or prevents binding between IL-27 and IL-27Ra. In some embodiments of the compositions, methods, and uses described herein, the IL-27 inhibitor is an antibody or antigen-binding fragment thereof that binds or physically interacts with the heterodimeric IL-27 receptor, and reduces, impedes, or blocks downstream IL-27 signaling, such as, for example, transcription factor induction (e.g., NFIL3 or T-bet induction), IL-10 induction, histone acetylation at a sequence at the TIM-3 locus, TIM-3 mRNA or protein upregulation, and/or elicitation of a cellular response to IL-27. Exemplary assays to measure inhibition or reduction of downstream IL-27 signaling pathway activities are known to those of ordinary skill in the art and are provided herein in the Examples.

In some embodiments of the compositions, methods, and uses described herein, an IL-27 inhibitor or antagonist is a monoclonal antibody.

In some embodiments of the compositions, methods, and uses described herein, an IL-27 inhibitor or antagonist is an antibody fragment or antigen-binding fragment. The terms “antibody fragment,” “antigen binding fragment,” and “antibody derivative” as used herein, refer to a protein fragment that comprises only a portion of an intact antibody, generally including an antigen binding site of the intact antibody and thus retaining the ability to bind antigen, and as described elsewhere herein.

In some embodiments of the compositions, methods, and uses described herein, an IL-27 inhibitor or antagonist is a chimeric antibody derivative of an IL-27 antagonist antibody or antigen-binding fragment thereof.

The IL-27 inhibitor or antagonist antibodies and antigen-binding fragments thereof described herein can also be, in some embodiments, a humanized antibody derivative.

In some embodiments, the IL-27 inhibitor or antagonist antibodies and antigen-binding fragments thereof described herein, i.e., antibodies that are useful for decreasing T cell exhaustion, include derivatives that are modified, i.e., by the covalent attachment of any type of molecule to the antibody, provided that the covalent attachment does not prevent the antibody from binding to the target antigen, e.g., IL-27.

In some embodiments of the compositions, methods, and uses described herein, completely human antibodies are used, which are particularly desirable for the therapeutic treatment of human patients.

In some embodiments of the compositions, methods, and uses described herein, an IL-27 inhibitor or antagonist is a small molecule inhibitor or antagonist, including, but is not limited to, small peptides or peptide-like molecules, soluble peptides, and synthetic non-peptidyl organic or inorganic compounds. A small molecule inhibitor or antagonist can have a molecular weight of any of about 100 to about 20,000 daltons (Da), about 500 to about 15,000 Da, about 1000 to about 10,000 Da. In some embodiments of the compositions, methods, and uses described herein, an IL-27 inhibitor or antagonist comprises a small molecule that binds IL-27. Exemplary sites of small molecule binding include, but are not limited to, the portion of IL-27 that binds to the IL-27 receptor, to IL-27Ra or to the portions of IL-27 adjacent to the IL-27 receptor binding region and which are responsible in whole or in part for establishing and/or maintaining the correct three-dimensional conformation of the receptor binding portion of IL-27. In some embodiments of the compositions, methods, and uses described herein, an IL-27 inhibitor or antagonist comprises a small molecule that binds to the IL-27 receptor or to IL-27Ra and inhibits an IL-27 biological activity. Exemplary sites of small molecule binding include, but are not limited to, those portions of the IL-27 receptor and/or IL-27Ra that bind to IL-27.

In some embodiments of the compositions, methods, and uses described herein, an IL-27 inhibitor or antagonist is an RNA or DNA aptamer that binds or physically interacts with IL-27, and blocks interactions between IL-27 and its receptor. In some embodiments of the cocompositions, methods, and uses described herein, the aptamer comprises at least one RNA or DNA aptamer that binds to the p28 subunit of IL-27. In some embodiments of the compositions, methods, and uses described herein, the aptamer comprises at least one RNA or DNA aptamer that binds to the Ebi3 subunit of IL-27. In some embodiments of the compositions, methods, and uses described herein, an IL-27 inhibitor or antagonist comprises at least one RNA or DNA aptamer that binds to both subunits of IL-27. In some embodiments of the compositions, methods, and uses described herein, an IL-27 inhibitor or antagonist is an RNA or DNA aptamer that binds or physically interacts with the heterodimeric IL-27 receptor or the IL-27Ra subunit, and reduces, impedes, or blocks downstream IL-27 signaling.

In some embodiments of the compositions, methods, and uses described herein, an IL-27 inhibitor or antagonist comprises at least one IL-27 or IL-27 receptor structural analog. The terms IL-27 structural analogs and IL-27 receptor structural analogs, as used herein, refer to compounds that have a similar three dimensional structure as part of that of IL-27 or IL-27 receptor, or IL-27Ra and which bind to IL-27 (e.g., IL-27 receptor or IL-27Ra structural analogs) or to IL-27 receptor (e.g., IL-27, IL-27p28, and IL-27Ebi3 structural analogs) under physiological conditions in vitro or in vivo, wherein the binding at least partially inhibits an IL-27 biological activity or an IL-27 receptor biological activity, such as NFIL-3 or TIM-3 induction. Suitable IL-27 structural analogs and IL-27 receptor structural analogs can be designed and synthesized through molecular modeling of IL-27 receptor binding. The IL-27 structural analogs and IL-27 receptor structural analogs can be monomers, dimers, or higher order multimers in any desired combination of the same or different structures to obtain improved affinities and biological effects.

In some embodiments of the compositions, methods, and uses described herein, an IL-27 inhibitor or antagonist comprises at least one soluble IL-27 receptor (e.g., IL-27Ra) or fusion polypeptide thereof. In some such embodiments, the soluble IL-27Ra is fused to an immunoglobulin constant domain, such as an Fc domain.

In some embodiments of the compositions, methods, and uses described herein, an IL-27 inhibitor or antagonist comprises at least one antisense molecule capable of blocking or decreasing the expression of functional IL-27 or IL-27 receptor by targeting nucleic acids encoding a subunit of IL-27 (i.e., IL-27p28 or IL-27Ebi3), or IL-27Ra. Nucleotide sequences of IL-27 and IL-27 receptor are known. See, for example, e.g., GenBank Accession Nos. NM 005755 (human IL-27Ebi3 mRNA); NM 145659 (human IL-27p28 mRNA); and NM 004843 (human IL-27Ra mRNA). Methods are known to those of ordinary skill in the art for the preparation of antisense oligonucleotide molecules that will specifically bind one or more of IL-27p28, IL-27Ebi3, and IL-27Ra mRNA without cross-reacting with other polynucleotides. Exemplary sites of targeting include, but are not limited to, the initiation codon, the 5′ regulatory regions, including promoters or enhancers, the coding sequence, including any conserved consensus regions, and the 3′ untranslated region. In some embodiment of these aspects and all such aspects described herein, the antisense oligonucleotides are about 10 to about 100 nucleotides in length, about 15 to about 50 nucleotides in length, about 18 to about 25 nucleotides in length, or more. In certain embodiments, the oligonucleotides further comprise chemical modifications to increase nuclease resistance and the like, such as, for example, phosphorothioate linkages and 2′-O-sugar modifications known to those of ordinary skill in the art.

In some embodiments of the compositions, methods, and uses described herein, an IL-27 inhibitor or antagonist comprises at least one siRNA molecule capable of blocking or decreasing the expression of functional IL-27 or IL-27 receptor by targeting nucleic acids encoding IL-27, a subunit of IL-27 (i.e., IL-27p28 or IL-27Ebi3), or IL-27Ra. It is routine to prepare siRNA molecules that will specifically target one or more of IL-27p28, IL-27Ebi3, and IL-27Ra mRNA without cross-reacting with other polynucleotides. siRNA molecules for use in the compositions, methods, and uses described herein can be generated by methods known in the art, such as by typical solid phase oligonucleotide synthesis, and often will incorporate chemical modifications to increase half life and/or efficacy of the siRNA agent, and/or to allow for a more robust delivery formulation. Alternatively, siRNA molecules are delivered using a vector encoding an expression cassette for intracellular transcription of siRNA.

IL-27 inhibitors or antagonists for use in the compositions, methods, and uses described herein can be identified or characterized using methods known in the art, such as protein-protein binding assays, biochemical screening assays, immunoassays, and cell-based assays, which are well known in the art, including, but not limited to, those described herein in the Examples.

For example, to identify a molecule that inhibits interaction between IL-27 and its receptor, binding assays can be used. For example, IL-27 or receptor polypeptide is immobilized on a microtiter plate by covalent or non-covalent attachment. The assay is performed by adding the non-immobilized component (ligand or receptor polypeptide), which can be labeled by a detectable label, to the immobilized component, in the presence or absence of the testing molecule. When the reaction is complete, the non-reacted components are removed and binding complexes are detected. If formation of binding complexes is inhibited by the presence of the testing molecule, the testing molecule can be deemed a candidate antagonist that inhibits binding between IL-27 and its receptor. Cell-based or membrane-based assays can also be used to identify IL-27 antagonists. For example, IL-27 can be added to a cell along with the testing molecule to be screened for a particular activity (e.g., induction of NFIL-3 or TIM-3), and the ability of the testing molecule to inhibit the activity of interest indicates that the testing molecule is an IL-27 antagonist. In other embodiments, by detecting and/or measuring levels of IL-27 gene expression, antagonist molecules that inhibit IL-27 gene expression can be tested. IL-27 gene expression can be detected and/or measured by a variety of methods, such as real time RT-PCR, enzyme-linked immunosorbent assay (“ELISA”), Northern blotting, or flow cytometry, and as known to one of ordinary skill in the art.

Also provided herein, in other aspects, are compositions comprising IL-27 activators or agonists for use in increasing T cell exhaustion by increasing or promoting TIM-3 induction and/or activity.

As used herein, the terms “IL-27 activator,” “IL-27 agonist,” IL-27 activator agent,” and “IL-27 agonist agent” refer to a molecule or agent that mimics or up-regulates (e.g., increases, potentiates or supplements) the expression and/or biological activity of IL-27 in vitro, in situ, and/or in vivo, including downstream pathways mediated by IL-27 signaling, such as, for example, transcription factor induction (e.g., NFIL3 or T-bet induction), IL-10 induction, histone acetylation at the TIM-3 locus, TIM-3 mRNA or protein upregulation, and/or elicitation of a cellular response to IL-27. An IL-27 activator or agonist can be a wild-type IL-27 protein or derivative thereof having at least one bioactivity of the wild-type IL-27. An IL-27 activator or agonist can also be a compound that up-regulates expression of IL-27 or its subunits. An IL-27 activator or agonist can also be a compound which increases the interaction of IL-27 with its receptor, for example. Exemplary IL-27 activators or agonists contemplated for use in the various aspects and embodiments described herein include, but are not limited to, anti-IL-27 antibodies or antigen-binding fragments thereof that specifically bind to IL-27 or one or both subunits of IL-27 (i.e., IL-27p28 and/or EBI3/IL27B), and/or bind to IL-27 bound to the IL-27R; RNA or DNA aptamers that bind to the IL-27Ra and mimic IL-27 binding to IL-27R; IL-27 structural analogs or soluble IL-27 mimics or fusion polypeptides thereof; and small molecule agents that target or bind to IL-27 or the IL27R and act as functional mimics. In some embodiments of these aspects and all such aspects described herein, an IL-27 activator or agonist (e.g., an antibody or antigen-binding fragment thereof) selectively binds (physically interacts with) binds to an IL-27Ra, and increases (activates/enhances) downstream IL-27Ra signaling, and/or increases or up-regulates IL-27 synthesis, production or release. In some embodiments of these aspects and all such aspects described herein, an IL-27 activator or agonist increases or enhances expression (i.e., transcription or translation) of IL-27, an IL-27 subunit, or IL-27Ra.

As used herein, an IL-27 agonist has the ability to increase or enhance the activity and/or expression of IL-27 in a cell (e.g., T cells, such as CD8+ or CD4+ T cells) by at least 5%, at least 10%, at least 20%, at least 30%, at least 40%, at least 50%, at least 60%, at least 70%, at least 80%, at least 90%, at least 95%, at least 98%, at least 99%, at least 100%, at least 1.5-fold, at least 2-fold, at least 5-fold, at least 10-fold, at least 25-fold, at least 50-fold, at least 100-fold, at least 1000-fold, or more relative to the activity or expression level in the absence of the IL-27 activator or agonist.

In some embodiments of the compositions, methods, and uses described herein, the IL-27 activator or agonist increases or enhances IL-27 mediated signal transduction. In some embodiments of the compositions and methods described herein, the IL-27 activator or agonist increases or enhances IL-27-mediated transcription factor induction or activation, for example, NFIL3 or T-bet induction or activation. In some embodiments of the compositions, methods, and uses described herein, the IL-27 activator or agonist increases or enhances IL-27-mediated NFIL-3 binding to conserved cis-regulatory regions or sequences at the TIM-3 locus, such as, for example, a sequence selected from any one of SEQ ID NO: 46-SEQ ID NO: 70. In some embodiments of the compositions and methods described herein, the IL-27 activator or agonist increases or enhances IL-27-mediated histone acetylation at a sequence at the TIM-3 locus, such as histone acetylation at intron 1. In some embodiments of the compositions, methods, and uses described herein, the IL-27 activator or agonist increases or enhances IL-27-mediated TIM-3 mRNA or protein upregulation. In some embodiments of the compositions, methods, and uses described herein, the IL-27 activator or agonist increases or enhances IL-27-induced IL-10 production.

In some embodiments of the compositions, methods, and uses described herein, the IL-27 activator or agonist is an antibody or antigen-binding fragment thereof that selectively binds or physically interacts with a subunit of IL-27 (IL-27p28 or IL-27Ebi3), and enhances or increases formation of the heterodimeric IL-27.

In some embodiments of the compositions, methods, and uses described herein, the binding sites of the IL-27 activators or agonists, such as an antibody or antigen-binding fragment thereof, are directed against an IL-27R ligand interaction site. By binding to an IL-27 ligand interaction site, an IL-27 activator or agonist described herein can mimic or recapitulate IL-27 binding to the receptor and increase the activity or expression of IL-27, and downstream IL-27 signaling consequences (e.g., transcription factor induction (e.g., NFIL3 or T-bet induction), IL-10 induction, histone acetylation at a sequence at the TIM-3 locus, TIM-3 mRNA or protein upregulation, and/or elicitation of a cellular response to IL-27).

In some embodiments of the compositions, methods, and uses described herein, the IL-27 activator or agonist is an antibody or antigen-binding fragment thereof that binds or physically interacts with IL-27Ra. In some embodiments of the compositions, methods, and uses described herein, the IL-27 activator or agonist is an antibody or antigen-binding fragment thereof that binds IL-27Ra and increases and/or promotes formation of heterodimeric IL-27 receptor. In some embodiments of the compositions, methods, and uses described herein, the IL-27 activator or agonist is an antibody or antigen-binding fragment thereof that binds IL-27Ra and increases and/or enhances binding between IL-27 and IL-27Ra. In some embodiments of the compositions, methods, and uses described herein, the IL-27 activator or agonist is an antibody or antigen-binding fragment thereof that binds or physically interacts with the heterodimeric IL-27 receptor, and mimics IL-27 binding and increases, upregulates, or enhances, downstream IL-27 signaling, such as, for example, transcription factor induction (e.g., NFIL3 or T-bet induction), IL-10 induction, histone acetylation at the TIM-3 locus, TIM-3 mRNA or protein upregulation, and/or elicitation of a cellular response to IL-27. Exemplary assays to measure increases or up-regulation of downstream IL-27 signaling pathway activities are known to those of ordinary skill in the art and are provided herein in the Examples.

In some embodiments of the compositions, methods, and uses described herein, an IL-27 activator or agonist is a monoclonal antibody. In some embodiments of the compositions, methods, and uses described herein, an IL-27 activator or agonist is an antibody fragment or antigen-binding fragment, as described in more detail elsewhere herein.

In some embodiments of the compositions, methods, and uses described herein, an IL-27 activator or agonist is a chimeric antibody derivative of the IL-27 agonist antibodies and antigen-binding fragments thereof, as described in more detail elsewhere herein.

In some embodiments of the compositions, methods, and uses described herein, an IL-27 activator or agonist is a humanized antibody derivative, as described in more detail elsewhere herein.

In some embodiments, the IL-27 activator or agonist antibodies and antigen-binding fragments thereof described herein, i.e., antibodies that are useful for increasing T cell exhaustion, include derivatives that are modified, i.e. , by the covalent attachment of any type of molecule to the antibody, provided that covalent attachment does not prevent the antibody from binding to the target antigen, e.g., IL-27.

The IL-27 activator or agonist antibodies and antigen-binding fragments thereof described herein for use in increasing or promoting T cell exhaustion by increasing TIM-3 induction or activity, as well as any of the other antibodies or antigen-binding fragments thereof described herein in various aspects and embodiments, can be generated by any suitable method known in the art.

In some embodiments, the IL-27 activator or agonist antibodies and antigen-binding fragments thereof described herein, i.e., antibodies that are useful for increasing T cell exhaustion, are completely human antibodies or antigen-binding fragments thereof, which are particularly desirable for the therapeutic treatment of human patients. Human antibodies can be made by a variety of methods known in the art, and as described in more detail elsewhere herein.

In some embodiments of the compositions, methods, and uses described herein, an IL-27 activator or agonist is a small molecule activator or agonist, including, but is not limited to, small peptides or peptide-like molecules, soluble peptides, and synthetic non-peptidyl organic or inorganic compounds. A small molecule activator or agonist can have a molecular weight of any of about 100 to about 20,000 daltons (Da), about 500 to about 15,000 Da, about 1000 to about 10,000 Da. In some embodiments of the compositions, methods, and uses described herein, an IL-27 activator or agonist comprises a small molecule that binds the IL-27R and mimics IL-27 binding. Exemplary sites of small molecule binding include, but are not limited to, the portion of the IL-27 receptor that binds to IL-27, to IL-27Ra or to the portions of IL-27 adjacent to the IL-27 receptor binding region and which are responsible in whole or in part for establishing and/or maintaining the correct three-dimensional conformation of the receptor binding portion of IL-27. In some embodiments of the compositions, methods, and uses described herein, an IL-27 activator or agonist comprises a small molecule that binds to the IL-27 receptor or to IL-27Ra and increases or promotes an IL-27 biological activity. Exemplary sites of small molecule binding include, but are not limited to, those portions of the IL-27 receptor and/or IL-27Ra that bind to IL-27.

In some embodiments of the compositions, methods, and uses described herein, an IL-27 activator or agonist is an RNA or DNA aptamer that binds or physically interacts with IL-27 or the IL-27 receptor, and enhances or promotes interactions between IL-27 and its receptor. In some embodiments of the compositions, methods, and uses described herein, the aptamer comprises at least one RNA or DNA aptamer that binds to the p28 subunit of IL-27. In some embodiments of the compositions, methods, and uses described herein, the aptamer comprises at least one RNA or DNA aptamer that binds to the Ebi3 subunit of IL-27. In some embodiments of the compositions, methods, and uses described herein, an IL-27 activator or agonist comprises at least one RNA or DNA aptamer that binds to both subunits of IL-27. In some embodiments of the compositions, methods, and uses described herein, an IL-27 activator or agonist is an RNA or DNA aptamer that binds or physically interacts with the heterodimeric IL-27 receptor or the IL-27Ra subunit, and increases, enhances, or promotes downstream IL-27 signaling.

In some embodiments of the compositions, methods, and uses described herein, an IL-27 activator or agonist comprises at least one IL-27 structural analog. The term IL-27 structural analog, as used herein, refer to compounds that have a similar three dimensional structure as part of that of IL-27 and which bind to IL-27 receptor (e.g., IL-27, IL-27p28, and IL-27Ebi3 structural analogs) under physiological conditions in vitro or in vivo, wherein the binding at least partially mimics or increases an IL-27 biological activity or an IL-27 receptor biological activity, such as NFIL-3 or TIM-3 induction. Suitable IL-27 structural analogs can be designed and synthesized through molecular modeling of IL-27 receptor binding. The IL-27 structural analogs can be monomers, dimers, or higher order multimers in any desired combination of the same or different structures to obtain improved affinities and biological effects.

IL-27 activators or agonists for use in the compositions, methods, and uses described herein can be identified or characterized using methods known in the art, such as protein-protein binding assays, biochemical screening assays, immunoassays, and cell-based assays, which are well known in the art, such as those described herein in the Examples.

For example, to identify a molecule that increases interaction between IL-27 and its receptor, binding assays can be used. For example, IL-27 or receptor polypeptide is immobilized on a microtiter plate by covalent or non-covalent attachment. The assay is performed by adding the non-immobilized component (ligand or receptor polypeptide), which can be labeled by a detectable label, to the immobilized component, in the presence or absence of the testing molecule. When the reaction is complete, the non-reacted components are removed and binding complexes are detected. If formation of binding complexes is enhanced or increased by the presence of the testing molecule, the testing molecule can be a candidate activator or agonist that increases or promotes binding between IL-27 and its receptor. Cell-based assays can also be used to identify IL-27 activators or agonists. For example, the candidate agent can be added to a cell alone or in the presence of IL-27 to be screened for a particular activity (e.g., induction of NFIL-3 or TIM-3), and the ability of the candidate to increase the activity of interest or to mimic IL-27 binding indicates that the testing molecule is an IL-27 activator or agonist. In other embodiments, by detecting and/or measuring levels of IL-27 gene expression, activator or agonist molecules that increase IL-27 gene expression can be tested. IL-27 gene expression can be detected and/or measured by a variety of methods, such as real time RT-PCR, enzyme-linked immunosorbent assay (“ELISA”), Northern blotting, or flow cytometry, and as known to one of ordinary skill in the art.

As used herein, in regard to an IL-27 modulator, “selectively binds” or “specifically binds” or “specific for” refers to the ability of an IL-27 inhibitor/antagonist or IL-27 activator/agonist as described herein , such as, for example, an IL-27 antagonist antibody or IL-27 antigen-binding fragment thereof, to bind to a target, such as IL-27, IL-27p28, IL-27Ebi3, IL-27 receptor, or IL-27Ra, with a K_{D} 10⁻⁵ M (10000 nM) or less, e.g., 10⁻⁶ M or less, 10⁻⁷ M or less, 10⁻⁸ M or less, 10⁻⁹ M or less, 10⁻¹⁰ M or less, 10⁻¹¹M or less, or 10⁻¹² M or less. For example, if an IL-27 inhibitor/antagonist or an IL-27 activator/agonist described herein binds to IL-27 with a K_{D} of 10⁻⁵ M or lower, but not to a related cytokine, sharing, for example, the IL-27Ebi3 subunit, then the agent is said to specifically bind IL-27. Specific binding can be influenced by, for example, the affinity and avidity of, for example, the IL-27 inhibitor/antagonist or IL-27 activator/agonist antibody or antigen-binding fragment thereof and the concentration of polypeptide agent. The person of ordinary skill in the art can determine appropriate conditions under which the polypeptide agents described herein selectively bind the targets using any suitable methods, such as titration of a polypeptide agent in a suitable cell binding assay.

Antibodies specific for or that selectively bind IL-27 or IL-27Ra, whether an IL-27 activator/agonist antibody or IL-27 blocking or antagonist antibody, suitable for use in the compositions and for practicing the methods described herein are preferably monoclonal, and can include, but are not limited to, human, humanized or chimeric antibodies, comprising single chain antibodies, Fab fragments, F(ab′) fragments, fragments produced by a Fab expression library, and/or binding fragments of any of the above. Antibodies also refer to immunoglobulin molecules and immunologically active portions of immunoglobulin molecules, i.e., molecules that contain antigen or target binding sites or “antigen-binding fragments.” The immunoglobulin molecules described herein can be of any type (e.g., IgG, IgE, IgM, IgD, IgA and IgY), class (e.g., IgG1, IgG2, IgG3, IgG4, IgA1 and IgA2) or subclass of immunoglobulin molecule, as is understood by one of skill in the art.

In some embodiments of the compositions, methods, and uses described herein, an IL-27 inhibitor/antagonist or IL-27 activator/agonist as described herein is a monoclonal IL-27 antibody fragment or antigen-binding fragment.

In some embodiments of the compositions, methods, and uses described herein, an IL-27 inhibitor/antagonist or IL-27 activator/agonist as described herein is an IL-27 antibody fragment or antigen-binding fragment. Examples of antibody fragments encompassed by the terms antibody fragment or antigen-binding fragment include: (i) the Fab fragment, having V_{L}, C_{L}, V_{H} and C_{H}1 domains; (ii) the Fab′ fragment, which is a Fab fragment having one or more cysteine residues at the C-terminus of the C_{H} 1 domain; (iii) the Fd fragment having V_{H} and C_{H}1 domains; (iv) the Fd′ fragment having V_{H} and C_{H}1 domains and one or more cysteine residues at the C-terminus of the CH1 domain; (v) the Fv fragment having the V_{L} and V_{H} domains of a single arm of an antibody; (vi) a dAb fragment (Ward et al., Nature 341, 544-546 (1989)) which consists of a V_{H} domain or a V_{L} domain ; (vii) isolated CDR regions; (viii) F(ab′)₂ fragments, a bivalent fragment including two Fab′ fragments linked by a disulphide bridge at the hinge region; (ix) single chain antibody molecules (e.g. single chain Fv; scFv) (Bird et al., Science 242:423-426 (1988); and Huston et al., PNAS (USA) 85:5879-5883 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; and Hollinger et al., Proc. Natl. Acad. Sci. USA, 90:6444-6448 (1993)); (xi) “linear antibodies” comprising a pair of tandem Fd segments (V_{H}-C_{H}1-V_{H}-C_{H}1) which, together with complementary light chain polypeptides, form a pair of antigen binding regions (Zapata et al. Protein Eng. 8(10):1057-1062 (1995); and U.S. Pat. No. 5,641,870); and modified versions of any of the foregoing (e.g., modified by the covalent attachment of polyalkylene glycol (e.g., polyethylene glycol, polypropylene glycol, polybutylene glycol) or other suitable polymer).

NFIL-3 (Nuclear factor interleukin-3-regulated protein, also known as E4BP4; IL3BP1; NFIL3A; NF-IL3A) acts as a transcriptional regulator that recognizes and binds to the sequence 5′-[GA]TTA[CT]GTAA[CT]-3′ (SEQ ID NO: 4), a sequence present in many cellular and viral promoters. NFIL-3 is known to repress transcription from promoters with activating transcription factor (ATF) sites, and activates transcription from the interleukin-3 promoter in T-cells. NFIL-3 is reported to be a component of the circadian clock that acts as a negative regulator for the circadian expression of PER2 oscillation in the cell autonomous core clock, and protects pro-B cells from programmed cell death.

Demonstrated herein for the first time is a role for NFIL-3 in inducing expression and activity of the inhibitory molecule TIM-3 and consequent role in induction of T cell functional exhaustion. Ectopic expression of NFIL3 in T cells via retrovirus, and consequent increased expression of TIM-3, resulted in potent suppressive effects and induces exhaustion-like phenotypes in T cells, and reduced colitis severity, while NFIL3 deficiency in T cells resulted in reduced numbers of T cells with an exhausted phenotype. It was also demonstrated that NFIL-3 binds to a sequence at the TIM-3 proximal promoter region and/or a sequence at intron 1 of the TIM-3 locus and/or a sequence at intron 3 of the TIM-3 locus, and/or a sequence at intron 5 of the TIM-3 locus, and that NFIL-3 regulates histone acetylation at a sequence at the TIM-3 locus, such as at intron 1. Accordingly, provided herein are novel compositions, methods, and uses to modulate chronic immune conditions by inhibiting or activating NFIL-3 to modulate TIM-3 expression and/or activity, and resulting suppression/activation of immune responses or development of T cell exhaustion phenotypes.

The term “NFIL-3” as used herein, refers to the 462 amino acid polypeptide having the amino acid sequence: MQLRKMQTVKKEQASLDASSNVDKMMVLNSALTEVSEDSTTGEELLLSEGSVGKNKSSAC RRKREFIPDEKKDAMYWEKRRKNNEAAKRSREKRRLNDLVLENKLIALGEENATLKAELLS LKLKFGLISSTAYAQEIQKLSNSTAVYFQDYQTSKSNVSSFVDEHEPSMVSSSCISVIKHSPQSS LSDVSEVSSVEHTQESSVQGSCRSPENKFQIIKQEPMELESYTREPRDDRGSYTASIYQNYMG NSFSGYSHSPPLLQVNRSSSNSPRTSETDDGVVGKSSDGEDEQQVPKGPIHSPVELKHVHATV VKVPEVNSSALPHKLRIKAKAMQIKVEAFDNEFEATQKLSSPIDMTSKRHFELEKHSAPSMV HSSLTPFSVQVTNIQDWSLKSEHWHQKELSGKTQNSFKTGVVEMKDSGYKVSDPENLYLKQ GIANLSAEVVSLKRLIATQPISASDSG (SEQ ID NO: 5), as described by, e.g., NP_005375.2, together with any naturally occurring allelic, splice variants, and processed forms thereof. Typically, NFIL-3 refers to human NFIL-3.

The term “NFIL-3” is also used to refer to truncated forms or fragments of the NFIL-3 polypeptide having transcription factor activity, for example. Reference to any such forms or fragments of NFIL-3 can be identified in the application, e.g., by “NFIL-3 (72-123)” (which encodes the leucine zipper domain). Specific residues of TIM-3 can be referred to as, for example, “NFIL-3(301)” or “NFIL-3(353),” which are phosphorylation sites.

Accordingly, also provided herein, in some aspects, are compositions comprising NFIL-3 inhibitors or antagonists for use in decreasing T cell exhaustion by inhibiting TIM-3 induction and/or activity.

As used herein, the terms “NFIL-3 inhibitor,” “NFIL-3 antagonist,” “NFIL-3 inhibitor agent,” or “NFIL-3 antagonist agent” refer to a molecule or agent that blocks, inhibits, reduces (including significantly), or interferes with NFIL-3 (mammalian, such as human NFIL-3) biological activity in vitro, in situ, and/or in vivo. An NFIL-3 inhibitor will block or inhibit NFIL-3 biological activity, including, for example, NFIL-3's activity on, for example, cytokine induction (e.g., IL-10 induction), NFIL-3 binding to a sequence at the TIM-3 proximal promoter region, such as, for example, a sequence selected from any one of SEQ ID NO: 46-SEQ ID NO: 70, and/or a sequence at intron 1 of the TIM-3 locus and/or a sequence at intron 3 of the TIM-3 locus, and/or a sequence at intron 5 of the TIM-3 locus; histone acetylation at a sequence at the TIM-3 locus, such as at intron 1; TIM-3 mRNA or protein upregulation, etc. Exemplary NFIL-3 inhibitors or antagonists contemplated for use in the various aspects and embodiments described herein include, but are not limited to, anti-NFIL-3 antibodies or antigen-binding fragments thereof that specifically bind to NFIL-3; anti-sense molecules directed to a nucleic acid encoding NFIL-3; short interfering RNA (“siRNA”) molecules directed to a nucleic acid encoding NFIL-3; RNA or DNA aptamers that bind to NFIL-3; and small molecule compounds or agents that inhibit NFIL-3 or prevent NFIL-3 binding to promoter regions, such as a sequence at the TIM-3 locus promoter region. In some embodiments of these aspects and all such aspects described herein, a NFIL-3 antagonist (e.g., an antibody or antigen-binding fragment thereof, or small molecule agent) binds (physically interacts with) NFIL-3, and reduces (impedes and/or blocks) downstream effects of NFIL-3 activity, and/or inhibits (reduces) NFIL-3 synthesis, production or release or nuclear localization. In some embodiments of these aspects and all such aspects described herein, an NFIL-3 antagonist reduces or eliminates expression (i.e., transcription or translation) of NFIL-3.

In some embodiments of the compositions, methods, and uses described herein, the NFIL-3 inhibitor or antagonist inhibits NFIL-3 transcriptional activity, such as binding to promoter regions and/or increasing histone acetylation. In some embodiments of the compositions, methods, and uses described herein, the NFIL-3 inhibitor or antagonist inhibits NFIL-3 binding to conserved cis-regulatory regions or sequences at the TIM-3 locus, such as, for example, a sequence selected from any one of SEQ ID NO: 46-SEQ ID NO: 70. In some such embodiments, the NFIL-3 inhibitor or antagonist inhibits or reduces NFIL-3 binding to a sequence at the TIM-3 proximal promoter region and/or a sequence at intron 1 of the TIM-3 locus and/or a sequence at intron 3 of the TIM-3 locus, and/or a sequence at intron 5 of the TIM-3 locus. In some embodiments of the compositions, methods, and uses described herein, the NFIL-3 inhibitor or antagonist inhibits NFIL-3 mediated histone acetylation at a sequence at the TIM-3 locus, such as histone acetylation at intron 1. In some embodiments of the compositions, methods, and uses described herein, the NFIL-3 inhibitor or antagonist inhibits NFIL-3 induced TIM-3 mRNA or protein upregulation. In some embodiments of the compositions, methods, and uses described herein, the NFIL-3 inhibitor or antagonist inhibits NFIL-3 induced IL-10 production.

As used herein, an NFIL-3 inhibitor or antagonist has the ability to reduce the activity and/or expression of NFIL-3 in a cell (e.g., T cells, such as CD4+ or CD8+ T cells) by at least 5%, at least 10%, at least 20%, at least 30%, at least 40%, at least 50%, at least 60%, at least 70%, at least 80%, at least 90%, at least 95%, at least 98%, at least 99%, or more relative to the activity or expression level in the absence of the NFIL-3 inhibitor or antagonist.

In some embodiments of the compositions, methods, and uses described herein, the NFIL-3 inhibitor is an antibody or antigen-binding fragment thereof that selectively binds or physically interacts with NFIL-3. In some embodiments of the compositions, methods, and uses described herein, the NFIL-3 is an antibody or antigen-binding fragment thereof that selectively binds to the leucine zipper domain of NFIL-3 and inhibits and/or blocks and/or prevents binding of NFIL-3 to a target DNA sequence, such as a sequence at the TIM-3 proximal promoter region, such as, for example, a sequence selected from any one of SEQ ID NO: 46-SEQ ID NO: 70, and/or a sequence at intron 1 of the TIM-3 locus and/or a sequence at intron 3 of the TIM-3 locus, and/or a sequence at intron 5 of the TIM-3 locus. In some embodiments of the compositions, methods, and uses described herein, the NFIL-3 inhibitor is an antibody or antigen-binding fragment thereof that specifically binds to any of the phosphorylation sites of NFIL-3 and inhibits and/or blocks and/or prevents phosphorylation. In some embodiments of the compositions, methods, and uses described herein, the NFIL-3inhibitor is an antibody or antigen-binding fragment thereof that binds to NFIL-3 and inhibits and/or blocks and/or prevents nuclear localization of NFIL-3.

In some embodiments of the compositions, methods, and uses described herein, the binding sites of the NFIL-3 inhibitors, such as an antibody or antigen-binding fragment thereof, are directed against a DNA-binding site of NFIL-3. In some embodiments of the compositions, methods, and uses described herein, the binding sites of the NFIL-3 inhibitors are directed against a site on a target in the proximity of the DNA-binding site, in order to provide steric hindrance for the interaction of NFIL-3 with its target DNA sequence, such as, for example, a sequence selected from any one of SEQ ID NO: 46-SEQ ID NO: 70. By binding to an NFIL-3 DNA-binding site, a NFIL-3 inhibitor described herein can reduce or inhibit the activity or expression of NFIL-3, and downstream NFIL-3 consequences (e.g., IL-10 induction, histone acetylation at a sequence at the TIM-3 locus, TIM-3 mRNA or protein upregulation, and/or elicitation of a cellular response).

In some embodiments of the compositions, methods, and uses described herein, the NFIL-3 inhibitor is an antibody or antigen-binding fragment thereof that binds to the NFIL-3 bound to a target DNA sequence, such as, for example, a sequence selected from any one of SEQ ID NO: 46-SEQ ID NO: 70; such as a sequence at the TIM-3 proximal promoter region and/or a sequence at intron 1 of the TIM-3 locus and/or a sequence at intron 3 of the TIM-3 locus, and/or a sequence at intron 5 of the TIM-3 locus, but does not bind to either NFIL-3 or the target DNA sequence alone. In other words, in some embodiments of the compositions, methods, and uses described herein, the NFIL-3 inhibitor is an antibody or antigen-binding fragment thereof that binds to an epitope found in the NFIL-3 bound to a target DNA sequence, but not in either alone. In some embodiments of the compositions, methods, and uses described herein, the NFIL-3 inhibitor is an antibody or antigen-binding fragment thereof that binds or physically interacts with NFIL-3, and blocks interactions between NFIL-3 and its target DNA sequence, such as, for example, a sequence selected from any one of SEQ ID NO: 46-SEQ ID NO: 70, and reduces, impedes, or blocks downstream signaling consequences, such as, for example, IL-10 induction, histone acetylation at a sequence at the TIM-3 locus, TIM-3 mRNA or protein upregulation, and/or elicitation of a cellular response. Exemplary assays to measure inhibition or reduction of downstream NFIL-3 activities are known to those of ordinary skill in the art and are provided, for example, herein in the Examples.

In some embodiments of the compositions, methods, and uses described herein, a NFIL-3 inhibitor or antagonist is a monoclonal antibody. In some embodiments of the compositions, methods, and uses described herein, a NFIL-3 inhibitor or antagonist is an antibody fragment or antigen-binding fragment. In some embodiments of the compositions, methods, and uses described herein, an NFIL-3 inhibitor or antagonist is a chimeric antibody derivative of the NFIL-3 antagonist antibodies and antigen-binding fragments thereof The NFIL-3 inhibitor or antagonist antibodies and antigen-binding fragments thereof described herein can also be, in some embodiments, a humanized antibody derivative, as defined elsewhere herein. In some embodiments of the compositions, methods, and uses described herein, completely human NFIL-3 inhibitor antibodies are used, which are particularly desirable for the therapeutic treatment of human patients.

In some embodiments of the compositions, methods, and uses described herein, the NFIL-3 inhibitor or antagonist antibodies and antigen-binding fragments thereof described herein, i.e., antibodies that are useful for decreasing T cell exhaustion, include derivatives that are modified by the covalent attachment of any type of molecule to the antibody such that covalent attachment does not prevent the antibody from binding to NFIL-3.

In some embodiments of the compositions, methods, and uses described herein, a NFIL-3 inhibitor or antagonist is a small molecule inhibitor or antagonist, such as a small molecule compound or agent that inhibits NFIL-3 activity and/or prevents NFIL-3 binding to promoter regions, such as a sequence at the TIM-3 locus promoter region, and/or prevents NFIL-3-mediated histone acetylation. NFIL-3 small molecule inhibitors or antagonists include, but are not limited to, small peptides or peptide-like molecules, soluble peptides, and synthetic non-peptidyl organic or inorganic compounds. A small molecule inhibitor or antagonist can have a molecular weight of any of about 100 to about 20,000 daltons (Da), about 500 to about 15,000 Da, about 1000 to about 10,000 Da. In some embodiments of the compositions, methods, and uses described herein, a NFIL-3 inhibitor or antagonist comprises a small molecule that selectively binds a target site in the NFIL-3 molecule. Exemplary sites of small molecule binding include, but are not limited to, the portion of NFIL-3 that binds to target DNA sequences, the leucine zipper domain of NFIL-3, or any of the phosphorylation sites of NFIL-3, for example. Accordingly, in some embodiments of the compositions, methods, and uses described herein, the NFIL-3 inhibitor is a small molecule inhibitor thereof that selectively binds or physically interacts with NFIL-3. In some embodiments of the compositions, methods, and uses described herein, the NFIL-3 inhibitor is a small molecule inhibitor that selectively binds to the leucine zipper domain of NFIL-3 and inhibits and/or blocks and/or prevents binding of NFIL-3 to a target DNA sequence, such as, for example, a sequence selected from any one of SEQ ID NO: 46-SEQ ID NO: 70; such as a sequence at the TIM-3 proximal promoter region and/or a sequence at intron 1 of the TIM-3 locus and/or a sequence at intron 3 of the TIM-3 locus, and/or a sequence at intron 5 of the TIM-3 locus. In some embodiments of the compositions, methods, and uses described herein, the small molecule specifically binds to any of the phosphorylation sites of NFIL-3 and inhibits and/or blocks and/or prevents phosphorylation of NFIL-3. In some embodiments of the compositions, methods, and uses described herein, the small molecule inhibitor binds to NFIL-3 and inhibits and/or blocks and/or prevents nuclear localization of NFIL-3.

In some embodiments of the compositions, methods, and uses described herein, a NFIL-3 inhibitor or antagonist is an RNA or DNA aptamer that binds or physically interacts with NFIL-3, and blocks interactions between NFIL-3 and its target DNA sequence, such as, for example, a sequence selected from any one of SEQ ID NO: 46-SEQ ID NO: 70. In some embodiments of the compositions, methods, and uses described herein, the aptamer comprises at least one RNA or DNA aptamer that binds to the leucine zipper of NFIL-3. In some embodiments of the compositions, methods, and uses described herein, the aptamer comprises at least one RNA or DNA aptamer that binds to any of the phosphorylation sites of NFIL-3.

In some embodiments of the compositions, methods, and uses described herein, a NFIL-3 inhibitor or antagonist comprises at least one antisense molecule capable of blocking or decreasing the expression of functional NFIL-3 by targeting nucleic acids encoding NFIL-3, such as NM_005384.2. Methods are known to those of ordinary skill in the art for the preparation of antisense oligonucleotide molecules that will specifically bind a sequence encoding NFIL-3 without cross-reacting with other polynucleotides. In some embodiments of the compositions, methods, and uses described herein, the NFIL-3 inhibitor or antagonist is an anti-sense molecule directed to a nucleic acid encoding NFIL-3. Exemplary sites of targeting include, but are not limited to, the initiation codon, the 5′ regulatory regions, including promoters or enhancers, the coding sequence, including any conserved consensus regions, and the 3′ untranslated region. In one embodiment of these aspects and all such aspects described herein, the antisense oligonucleotides are about 10 to about 100 nucleotides in length, about 15 to about 50 nucleotides in length, about 18 to about 25 nucleotides in length, or more. In certain embodiments, the oligonucleotides further comprise chemical modifications to increase nuclease resistance and the like, such as, for example, phosphorothioate linkages and 2′-O-sugar modifications known to those of ordinary skill in the art.

In some embodiments of the compositions, methods, and uses described herein, a NFIL-3 inhibitor or antagonist comprises at least one siRNA molecule capable of blocking or decreasing the expression of functional NFIL-3 by targeting nucleic acids encoding NFIL-3, such as NM_005384.2. It is routine to prepare siRNA molecules that will specifically target NFIL-3 mRNA without cross-reacting with other polynucleotides. In some embodiments of the compositions, methods, and uses described herein, the NFIL-3 inhibitor or antagonist is a short interfering RNA (“siRNA”) molecule directed to a nucleic acid encoding NFIL-3.

NFIL-3 inhibitors or antagonists for use in the compositions, methods, and uses described herein can be identified or characterized using methods known in the art, such as protein-nucleic acid binding assays, reporter assays, histone acetylation assays, biochemical screening assays, immunoassays, and cell-based assays, which are well known in the art, such as those described herein in the Examples. For example, to identify a molecule that inhibits interaction between NFIL-3 and its target DNA sequence, or to identify a molecule that inhibits histone deacetylation, chromatin immunoprecipitation (ChIP) assays can be used, as described herein in the examples. Cell-based assays can also be used to identify NFIL-3 antagonists. In other embodiments, by detecting and/or measuring levels of NFIL-3 gene expression, antagonist molecules that inhibit NFIL-3 gene expression can be tested. NFIL-3 gene expression can be detected and/or measured by a variety of methods, such as real time RT-PCR, enzyme-linked immunosorbent assay (“ELISA”), Northern blotting, or flow cytometry, and as known to one of ordinary skill in the art.

Also provided herein, in other aspects, are compositions comprising NFIL-3 activators or agonists for use in increasing T cell exhaustion by increasing or promoting TIM-3 induction and/or activity.

As used herein, the terms “NFIL-3 activator,” “NFIL-3 agonist,” “NFIL-3 activator agent,” “NFIL-3 agonist agent” refer to a molecule or agent that mimics or up-regulates (e.g., increases, potentiates or supplements) the expression and/or biological activity of NFIL-3 in vitro, in situ, and/or in vivo. An NFIL-3 activator/agonist as described herein will modulate a biological activity modulated by NFIL-3 in the same direction (i.e., upregulated or downregulated) as NFIL-3 itself. Activities modulated by an NFI1-3 activator/agonist can include, for example, downstream pathways mediated by NFIL-3, such as, for example, IL-10 induction, histone acetylation at a sequence at the TIM-3 locus, TIM-3 mRNA or protein upregulation, and/or elicitation of a cellular response. An NFIL-3 activator or agonist can be a wild-type NFIL-3 protein or derivative thereof having at least one bioactivity of the wild-type NFIL-3. An NFIL-3 activator or agonist can also be a compound that up-regulates expression of NFIL-3. An NFIL-3 activator or agonist can also be a compound which increases the interaction of NFIL-3 with its target DNA sequence, . Exemplary NFIL-3 activators or agonists contemplated for use in the various aspects and embodiments described herein include, but are not limited to, anti-NFIL-3 antibodies or antigen-binding fragments thereof that specifically bind to NFIL-3 and potentiate its activity; RNA or DNA aptamers that bind to the NFIL-3 target DNA sequence and mimic NFIL-3 binding to its target DNA; NFIL-3 structural analogs or soluble NFIL-3 mimics or fusion polypeptides thereof; and small molecule agents that target or bind to NFIL-3 or NFIL-3 target DNA sequences and act as functional mimics. In some embodiments of these aspects and all such aspects described herein, an NFIL-3 activator or agonist (e.g., an antibody or antigen-binding fragment thereof) increases (activates/enhances) downstream NFIL-3 signaling consequences, such as IL-10 induction, histone acetylation at a sequence at the TIM-3 locus, and/or increases or up-regulates NFIL-3 synthesis, production or release. In some embodiments of these aspects and all such aspects described herein, a NFIL-3 activator or agonist increases or enhances expression (i.e., transcription or translation) of NFIL-3.

As used herein, an NFIL-3 agonist has the ability to increase or enhance the activity and/or expression of NFIL-3 in a cell (e.g., T cells, such as CD4+ or C84+ T cells) by at least 5%, at least 10%, at least 20%, at least 30%, at least 40%, at least 50%, at least 60%, at least 70%, at least 80%, at least 90%, at least 95%, at least 98%, at least 99%, at least 100%, at least 1.5-fold, at least 2-fold, at least 5-fold, at least 10-fold, at least 25-fold, at least 50-fold, at least 100-fold, at least 1000-fold, or more relative to the activity or expression level in the absence of the NFIL-3 activator or agonist.

In some embodiments of the compositions, methods, and uses described herein, the NFIL-3 activator or agonist increases or enhances NFIL-3 mediated signaling or transcriptional activity. In some embodiments of the compositions, methods, and uses described herein, the NFIL-3 activator or agonist increases or enhances NFIL-3 binding to conserved cis-regulatory regions in the TIM-3 locus. In some embodiments of the compositions, methods, and uses described herein, the NFIL-3 activator or agonist increases or enhances NFIL-3 mediated histone acetylation at a sequence at the TIM-3 locus, such as histone acetylation at intron 1. In some embodiments of the compositions, methods, and uses described herein, the NFIL-3 activator or agonist increases or enhances NFIL-3 mediated TIM-3 mRNA or protein upregulation. In some embodiments of the compositions, methods, and uses described herein, the NFIL-3 activator or agonist increases or enhances NFIL-3 mediated IL-10 production.

In some embodiments of the compositions, methods, and uses described herein, the binding sites of the NFIL-3 activators or agonists, are directed against a DNA target sequence, such as, for example, a sequence selected from any one of SEQ ID NO: 46-SEQ ID NO: 70. By binding to an NFIL-3 DNA target sequence, an NFIL-3 activator or agonist described herein can mimic or recapitulate NFIL-3 binding to its target DNA sequence and increase downstream NFIL-3 signaling consequences, e.g., IL-10 induction, histone acetylation at a sequence at the TIM-3 locus, TIM-3 mRNA or protein upregulation, and/or elicitation of a cellular response.

In some embodiments of the compositions, methods, and uses described herein, the NFIL-3 activator or agonist is an antibody or antigen-binding fragment thereof that binds or physically interacts with NFIL-3. In some embodiments of the compositions, methods, and uses described herein, the NFIL-3 activator or agonist is an antibody or antigen-binding fragment thereof that binds NFIL-3 and increases and/or promotes binding of NFIL-3 to a target DNA sequence, such as, for example, a sequence selected from any one of SEQ ID NO: 46-SEQ ID NO: 70. In some embodiments of the compositions, methods, and uses described herein, the NFIL-3 activator or agonist is an antibody or antigen-binding fragment thereof that binds or physically interacts with the NFIL-3 bound to its target DNA sequence, and increases and/or promotes binding and increases, upregulates, or enhances, downstream NFIL-3 signaling consequences, such as, for example, IL-10 induction, histone acetylation at a sequence at the TIM-3 locus, TIM-3 mRNA or protein upregulation, and/or elicitation of a cellular response. Exemplary assays to measure increases or up-regulation of downstream NFIL-3 signaling activities are known to those of ordinary skill in the art and are provided herein in the Examples.

In some embodiments of the compositions, methods, and uses described herein, a NFIL-3 activator or agonist is a monoclonal antibody. In some embodiments of the compositions, methods, and uses described herein, a NFIL-3 activator or agonist is an antibody fragment or antigen-binding fragment, as described in more detail elsewhere herein. In some embodiments of the compositions, methods, and uses described herein, a NFIL-3 activator or agonist is a chimeric antibody derivative of the NFIL-3 agonist antibodies and antigen-binding fragments thereof. In some embodiments of the compositions, methods, and uses described herein, a NFIL-3 activator or agonist is a humanized antibody derivative. In some embodiments, the NFIL-3 activator or agonist antibodies and antigen-binding fragments thereof described herein, i.e., antibodies that are useful for increasing T cell exhaustion, are completely human antibodies or antigen-binding fragments thereof. Human antibodies can be made by a variety of methods known in the art, and as described elsewhere herein.

In some embodiments, the NFIL-3 activator or agonist antibodies and antigen-binding fragments thereof described herein, i.e., antibodies that are useful for increasing T cell exhaustion, include derivatives that are modified, i.e. , by the covalent attachment of any type of molecule to the antibody, provided that the covalent attachment does not prevent the antibody from binding to, e.g., NFIL-3.

In some embodiments of the compositions, methods, and uses described herein, a NFIL-3 activator or agonist is a small molecule activator or agonist, including, but is not limited to, small peptides or peptide-like molecules, soluble peptides, and synthetic non-peptidyl organic or inorganic compounds. A small molecule activator or agonist can have a molecular weight of any of about 100 to about 20,000 daltons (Da), about 500 to about 15,000 Da, about 1000 to about 10,000 Da. In some embodiments of the compositions, methods, and uses described herein, a NFIL-3 activator or agonist comprises a small molecule that binds the NFIL-3 target DNA sequence and mimics NFIL-3 binding. Exemplary sites of small molecule binding include, but are not limited to, the portion of NFIL-3 that binds to target DNA sequences, the leucine zipper domain of NFIL-3, or any of the phosphorylation sites of NFIL-3, for example. Accordingly, in some embodiments of the compositions, methods, and uses described herein, the NFIL-3activator or agonist is a small molecule that selectively binds or physically interacts with NFIL-3. In some embodiments of the compositions, methods, and uses described herein, the NFIL-3 activator or agonist is a small molecule that selectively binds to the leucine zipper domain of NFIL-3 and/or increases or promotes binding of NFIL-3 to a target DNA sequence, such as, for example, a sequence selected from any one of SEQ ID NO: 46-SEQ ID NO: 70; such as a sequence at the TIM-3 proximal promoter region and/or a sequence at intron 1 of the TIM-3 locus and/or a sequence at intron 3 of the TIM-3 locus, and/or a sequence intron 5 of the TIM-3 locus. In some embodiments of the compositions, methods, and uses described herein, the small molecule activator or agonist specifically phosphorylates any of the phosphorylation sites of NFIL-3. In some embodiments of the compositions, methods, and uses described herein, the small molecule activator or agonist binds to NFIL-3 and increases or promotes nuclear localization of NFIL-3.

In some embodiments of the compositions, methods, and uses described herein, a NFIL-3 activator or agonist is an RNA or DNA aptamer that binds or physically interacts with a NFIL-3 DNA target sequence, and enhances or promotes downstream NFIL-3 signaling outcomes by mimicking NFIL-3 binding to an NFIL-3 target DNA sequence, such as, for example, a sequence selected from any one of SEQ ID NO: 46-SEQ ID NO: 70.

In some embodiments of the compositions, methods, and uses described herein, an NFIL-3 activator or agonist comprises at least one NFIL-3 structural analog. The term “NFIL-3 structural analog,” as used herein, refers to compounds that have a similar three dimensional structure as part of that of NFIL-3 and which bind to an NFIL-3 target DNA sequence(s) under physiological conditions in vitro or in vivo, wherein the binding at least partially mimics or increases an NFIL-3 biological activity, such as histone acetylation at a sequence at the TIM-3 locus, TIM-3 upregulation or IL-10 induction. Suitable IL-27 structural analogs can be designed and synthesized through molecular modeling of NFIL-3 binding to its target sequence.

NFIL-3 activators or agonists for use in the compositions, methods, and uses described herein can be identified or characterized using methods known in the art, such as protein-nucleic acid binding assays, reporter assays, histone acetylation assays, biochemical screening assays, immunoassays, and cell-based assays, which are well known in the art, such as those described herein in the Examples. For example, to identify a molecule that increases interaction between NFIL-3 and its target DNA sequence, or to identify a molecule that increases histone deacetylation, chromatin immunoprecipitation (ChIP) assays can be used, as described herein in the examples. Cell-based assays can also be used to identify NFIL-3 activators or agonists. In other embodiments, by detecting and/or measuring levels of NFIL-3 gene expression, antagonist molecules that increase NFIL-3 gene expression can be tested. NFIL-3 gene expression can be detected and/or measured by a variety of methods, such as real time RT-PCR, enzyme-linked immunosorbent assay (“ELISA”), Northern blotting, or flow cytometry, and as known to one of ordinary skill in the art.

As used herein, in regard to an NFIL3 modulator, “selectively binds” or “specifically binds” or “specific for” refer to the ability of an NFIL-3 inhibitor/antagonist or NFIL-3 activator/agonist as described herein, to bind to NFIL-3, with a K_{D} 10⁻⁵ M (10000 nM) or less, e.g., 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less. For example, if an NFIL-3inhibitor/antagonist or NFIL-3 activator/agonist described herein binds to NFIL-3 with a K_{D} of 10⁻⁵ M or lower, but not to a related transcription factor, then the agent is said to specifically bind NFIL-3. Specific binding can be influenced by, for example, the affinity and avidity of, for example, the NFIL-3 inhibitor/antagonist or activator/agonist antibody or antigen-binding fragment thereof and the concentration of polypeptide agent. The person of ordinary skill in the art can determine appropriate conditions under which the polypeptide agents described herein selectively bind the targets using any suitable methods, such as titration of a polypeptide agent in a suitable cell binding assay.

Antibodies specific for NFIL-3, whether inhibitor or antagonist or blocking or activator/agonist, suitable for use in the compositions and for practicing the methods described herein are preferably monoclonal, and can include, but are not limited to, human, humanized or chimeric antibodies, comprising single chain antibodies, Fab fragments, F(ab′) fragments, fragments produced by a Fab expression library, and/or binding fragments of any of the above. Antibodies also refer to immunoglobulin molecules and immunologically active portions of immunoglobulin molecules, i.e., molecules that contain antigen or target binding sites or “antigen-binding fragments.” The immunoglobulin molecules described herein can be of any type or subclass of immunoglobulin molecule, as is understood by one of skill in the art.

In some embodiments of the compositions, methods, and uses described herein, an NFIL-3 inhibitor/antagonist or NFIL-3 activator/agonist as described herein is a monoclonal NFIL-3 antibody fragment or antigen-binding fragment.

In some embodiments of the compositions, methods, and uses described herein, an NFIL-3 inhibitor/antagonist or NFIL-3 activator/agonist as described herein is an NFIL-3 antibody fragment or antigen-binding fragment. Examples of antibody fragments encompassed by the terms antibody fragment or antigen-binding fragment include: (i) the Fab fragment, having V_{L}, C_{L}, V_{H} and C_{H}1 domains; (ii) the Fab′ fragment, which is a Fab fragment having one or more cysteine residues at the C-terminus of the C_{H}1 domain; (iii) the Fd fragment having V_{H} and C_{H}1 domains; (iv) the Fd′ fragment having V_{H} and C_{H}1 domains and one or more cysteine residues at the C-terminus of the CH1 domain; (v) the Fv fragment having the V_{L} and V_{H} domains of a single arm of an antibody; (vi) a dAb fragment, which consists of a V_{H} domain or a V_{L} domain; (vii) isolated CDR regions; (viii) F(ab′)₂ fragments, a bivalent fragment including two Fab′ fragments linked by a disulphide bridge at the hinge region; (ix) single chain antibody molecules (e.g. single chain Fv; scFv); (x) “diabodies” with two antigen binding sites, comprising a heavy chain variable domain (V_{H}) connected to a light chain variable domain (V_{L}) in the same polypeptide chain; (xi) “linear antibodies” comprising a pair of tandem Fd segments (V_{H}-C_{H}1-V_{H}-C_{H}1) which, together with complementary light chain polypeptides, form a pair of antigen binding regions; and modified versions of any of the foregoing (e.g., modified by the covalent attachment of polyalkylene glycol (e.g., polyethylene glycol, polypropylene glycol, polybutylene glycol) or other suitable polymer).

Certain aspects described herein are based, in part, on the discovery by the inventors that IL-27 is a potent inducer of TIM-3 expression, and that IL-27-mediated induction of TIM-3 plays a critical role in functionally suppressing IFNy secreting T cells and T cell exhaustion during chronic immune conditions. While sustained TIM-3 expression has previously been shown to directly result in exhausted/dysregulated phenotype of antigen-specific T cells during chronic viral infections and cancers, little was known about the factors regulating TIM-3 expression.

As shown herein, in response to IL-27, transcription factors NFIL3 and T-bet synergistically activate TIM-3 expression. In addition, IL-27 signaling results in profound permissive chromatin remodeling of the TIM-3 locus, favoring TIM-3 transcription. Thus, IL-27 signaling suppresses Type I effector T cell function via induction of TIM-3 expression and other anti-inflammatory molecules, including IL-10. Further, as demonstrated herein, IL-27R deficient (WSX-1−/−) mice exhibit significant resistance to tumor growth that is accompanied by a failure to generate TIM-3+ exhausted T cells.

Also demonstrated herein for the first time is a role for NFIL-3 in inducing expression and activity of the inhibitory molecule TIM-3 and consequent role in induction of T cell functional exhaustion. Ectopic expression of NFIL-3 in T cells via retrovirus, and consequent increased expression of TIM-3, resulted in potent suppressive effects and induces exhaustion-like phenotypes in T cells, and reduced colitis severity, while NFIL-3 deficiency in T cells resulted in reduced numbers of T cells with an exhausted phenotype. It was also demonstrated that NFIL-3 binds to a sequence at the TIM-3 proximal promoter region and/or a sequence at intron 1 of the TIM-3 locus and/or a sequence at intron 3 of the TIM-3 locus, and/or a sequence at intron 5 of the TIM-3 locus, and that NFIL-3 regulates histone acetylation at a sequence at the TIM-3 locus, such as at intron 1.

Thus, the data provided herein identify IL-27 as a critical inducer of TIM-3 -mediated T cell exhaustion/dysfunction during chronic conditions, and demonstrate that this induction is mediated, in part, by transcription factor NFIL-3 induction. Accordingly, provided herein are novel compositions, methods, and uses to modulate chronic immune conditions by inhibiting or activating NFIL-3 to modulate TIM-3 expression and/or activity, and resulting suppression/activation of immune responses or development of T cell exhaustion phenotypes.

Accordingly, provided herein are methods for the treatment of chronic immune conditions, such as cancer, persistent infections, and autoimmune disorders in a subject in need thereof. These methods involve, in part, administering to a subject a therapeutically effective amount of an Il-27 or NFIL-3 modulating agent (i.e., activating or inhibiting) described herein. These methods are particularly aimed at therapeutic treatments of human subjects having a condition in which one or more immune cell populations, such as a CD4+ T cell population or a CD8+ T cell population, are functionally exhausted, and at therapeutic treatments of human subjects having a condition in which it is desired to cause or induce one or more immune cell populations, such as a CD4+ T cell population or a CD8+ T cell population, to become functionally exhausted.

Accordingly, provided herein, in some aspects are methods for the treatment of a chronic immune condition in a subject in need thereof, comprising administering to a subject an effective amount of a composition comprising an IL-27 inhibitor or antagonist that decreases T cell exhaustion by inhibiting TIM-3 induction and/or activity.

In some embodiments of these methods and all such methods described herein, the IL-27 inhibitor inhibits IL-27 mediated signal transduction. In some embodiments of these methods and all such methods described herein, the IL-27 inhibitor decreases or inhibits IL-27 mediated transcription factor induction or activation, for example, e.g., NFIL-3 or T-bet induction or activation. In some embodiments of these methods and all such methods described herein, the IL-27 inhibitor decreases or inhibits NFIL-3 binding to conserved cis-regulatory regions or sequences at the TIM-3 locus, such as, for example, a sequence selected from any one of SEQ ID NO: 46-SEQ ID NO: 70s. In some embodiments of these methods and all such methods described herein, the IL-27 inhibitor decreases or inhibits histone acetylation at a sequence at the TIM-3 locus, such as histone acetylation at a sequence at intron 1. In some embodiments of these methods and all such methods described herein, the IL-27 inhibitor decreases or inhibits IL-27 mediated TIM-3 mRNA or protein upregulation. In some embodiments of these methods and all such methods described herein, the IL-27 inhibitor decreases or inhibits IL-27-induced IL-10 production.

In some embodiments of these methods and all such methods described herein, the IL-27 inhibitor is an antibody or antigen-binding fragment thereof that selectively binds or physically interacts with a subunit of IL-27 (IL-27p28 or IL-27Ebi3). In some embodiments of these methods and all such methods described herein, the IL-27 inhibitor is an antibody or antigen-binding fragment thereof that binds to IL-27p28 or IL-27Ebi3 and inhibits and/or blocks and/or prevents formation of the heterodimeric IL-27. In some embodiments of these methods and all such methods described herein, the IL-27 inhibitor is an antibody or antigen-binding fragment thereof that binds to IL-27p28 and inhibits and/or blocks and/or prevents formation of the heterodimeric IL-27. In some embodiments of these methods and all such methods described herein, the IL-27 inhibitor is an antibody or antigen-binding fragment thereof that binds to IL-27Ebi3 and inhibits and/or blocks and/or prevents formation of the heterodimeric IL-27.

In some embodiments of these methods and all such methods described herein, the binding sites of the IL-27 inhibitors, such as an antibody or antigen-binding fragment thereof, are directed against an IL-27R ligand interaction site. In some embodiments of these methods and all such methods described herein, the binding sites of the IL-27 inhibitors are directed against a site on a target in the proximity of the ligand interaction site, in order to provide steric hindrance for the interaction of the target (e.g., IL-27) with its receptor (e.g., IL-27Ra).

In some embodiments of these methods and all such methods described herein, the IL-27 inhibitor is an antibody or antigen-binding fragment thereof that binds or physically interacts with IL-27Ra. In some embodiments of these methods and all such methods described herein, the IL-27 inhibitor is an antibody or antigen-binding fragment thereof that binds IL-27Ra and inhibits and/or prevents formation of heterodimeric IL-27 receptor. In some embodiments of these methods and all such methods described herein, the IL-27 inhibitor is an antibody or antigen-binding fragment thereof that binds IL-27Ra and inhibits and/or prevents binding between IL-27 and IL-27Ra. In some embodiments of these methods and all such methods described herein, the IL-27 inhibitor is an antibody or antigen-binding fragment thereof that binds or physically interacts with the heterodimeric IL-27 receptor, and reduces, impedes, or blocks downstream IL-27 signaling, such as, for example, transcription factor induction (e.g., NFIL-3 or T-bet induction), IL-10 induction, histone acetylation at the TIM-3 locus, TIM-3 mRNA or protein upregulation, and/or elicitation of a cellular response to IL-27.

In some embodiments of these methods and all such methods described herein, the IL-27 inhibitor or antagonist is an IL-27 specific monoclonal antibody. In some embodiments of these methods and all such methods described herein, an IL-27 inhibitor or antagonist is an antibody fragment or antigen-binding fragment, such as, for example: (i) the Fab fragment; (ii) the Fab′ fragment; (iii) the Fd; (iv) the Fd′ fragment; (v) the Fv fragment; (vi) the dAb fragment; (vii) isolated CDR regions; (viii) F(ab′)₂ fragments, a bivalent fragment including two Fab′ fragments linked by a disulphide bridge at the hinge region; (ix) single chain antibody molecules; (x) “diabodies” with two antigen binding sites; (xi) “linear antibodies”; and modified versions of any of the foregoing. In some embodiments of these methods and all such methods described herein, an IL-27 inhibitor or antagonist is a chimeric antibody derivative of the IL-27 antagonist antibodies and antigen-binding fragments thereof. In some embodiments of these methods and all such methods described herein, an IL-27 inhibitor or antagonist is a humanized or completely human anti-IL-27 antibody or antigen-binding fragment thereof.

In some embodiments of these methods and all such methods described herein, the IL-27 inhibitor is a small molecule compound or agent that targets or binds to IL-27, one or both subunits of IL-27, or to IL-27Ra. In some embodiments of these methods and all such methods described herein, an IL-27 inhibitor or antagonist comprises a small molecule that binds to the IL-27 receptor or to IL-27Ra and inhibits an IL-27 biological activity. Exemplary sites of small molecule binding include, but are not limited to, those portions of the IL-27 receptor and/or IL-27Ra that bind to IL-27.

In some embodiments of these methods and all such methods described herein, the IL-27 inhibitor is an RNA or DNA aptamer that binds to IL-27, one or both subunits of IL-27, or to IL-27Ra, and blocks interactions between IL-27 and its receptor. In some embodiments of these methods and all such methods described herein, the aptamer comprises at least one RNA or DNA aptamer that binds to the p28 subunit of IL-27. In some embodiments of these methods and all such methods described herein, the aptamer comprises at least one RNA or DNA aptamer that binds to the Ebi3 subunit of IL-27. In some embodiments of these methods and all such methods described herein, an IL-27 inhibitor or antagonist comprises at least one RNA or DNA aptamer that binds to both subunits of IL-27. In some embodiments of the compositions, methods, and uses described herein, an IL-27 inhibitor or antagonist is an RNA or DNA aptamer that binds or physically interacts with the heterodimeric IL-27 receptor or the IL-27Ra subunit, and reduces, impedes, or blocks downstream IL-27 signaling.

In some embodiments of these methods and all such methods described herein, an IL-27 inhibitor or antagonist comprises at least one IL-27 or IL-27 receptor structural analog.

In some embodiments of these methods and all such methods described herein, an IL-27 inhibitor or antagonist comprises at least one soluble IL-27 receptor (e.g., IL-27Ra) or fusion polypeptide thereof. In some such embodiments, the soluble IL-27Ra is fused to an immunoglobulin constant domain, such as an Fc domain.

In some embodiments of these methods and all such methods described herein, the IL-27 inhibitor is an anti-sense molecule directed to a nucleic acid encoding either subunit of IL-27 (i.e., IL-27p28 and/or EBI3/IL27B).

In some embodiments of these methods and all such methods described herein, the IL-27 inhibitor is a short interfering RNA molecule directed to a nucleic acid encoding acid encoding one or both subunits of IL-27 (i.e., IL-27p28 or IL-27Ebi3); or IL-27Ra3.

In some embodiments of these methods and all such methods described herein, the method further comprises administering any of the NFIL-3 inhibitor or antagonists described herein.

Also provided herein, in some aspects, are methods for the treatment of a chronic immune condition in a subject in need thereof, comprising administering to a subject in need thereof an effective amount of a composition comprising an NFIL-3 inhibitor or antagonist that decreases T cell exhaustion by inhibiting TIM-3 induction and/or activity.

In some embodiments of these methods and all such methods described herein, the NFIL-3 inhibitor or antagonist inhibits NFIL-3 transcriptional activity, such as binding to promoter regions and/or increasing histone acetylation and/or activating TIM-3 transcription. In some embodiments of these methods and all such methods described herein, the NFIL-3 inhibitor or antagonist inhibits NFIL-3 binding to conserved cis-regulatory regions or sequences at the TIM-3 locus, such as, for example, a sequence selected from any one of SEQ ID NO: 46-SEQ ID NO: 70. In some such embodiments, the NFIL-3 inhibitor or antagonist inhibits or reduces NFIL-3 binding to a sequence at the TIM-3 proximal promoter region and/or a sequence at intron 1 of the TIM-3 locus and/or a sequence at intron 3 of the TIM-3 locus, and/or a sequence at intron 5 of the TIM-3 locus. In some embodiments of these methods and all such methods described herein, the NFIL-3 inhibitor or antagonist inhibits histone acetylation at a sequence at the TIM-3 locus, such as histone acetylation at intron 1. In some embodiments of these methods and all such methods described herein, the NFIL-3 inhibitor or antagonist inhibits IL-27 mediated TIM-3 mRNA or protein upregulation. In some embodiments of these methods and all such methods described herein, the NFIL-3 inhibitor or antagonist inhibits IL-10 production.

In some embodiments of these methods and all such methods described herein, the NFIL-3 inhibitor or antagonist is an antibody or antigen-binding fragment thereof that specifically binds to or physically interacts with NFIL-3. In some embodiments of these methods and all such methods described herein, the NFIL-3 inhibitor is an antibody or antigen-binding fragment thereof that selectively binds or physically interacts with NFIL-3. In some embodiments of these methods and all such methods described herein, the NFIL-3 is an antibody or antigen-binding fragment thereof that selectively binds to the leucine zipper domain of NFIL-3 and inhibits and/or blocks and/or prevents binding of NFIL-3 to a target DNA sequence, such as, for example, a sequence selected from any one of SEQ ID NO: 46-SEQ ID NO: 70; such as a sequence at the TIM-3 proximal promoter region and/or a sequence at intron 1 of the TIM-3 locus and/or a sequence at intron 3 of the TIM-3 locus, and/or a sequence at intron 5 of the TIM-3 locus. In some embodiments of these methods and all such methods described herein, the NFIL-3 inhibitor is an antibody or antigen-binding fragment thereof that specifically binds to any of the phosphorylation sites of NFIL-3 and inhibits and/or blocks and/or prevents phosphorylation. In some embodiments of these methods and all such methods described herein, the NFIL-3inhibitor is an antibody or antigen-binding fragment thereof that binds to NFIL-3 and inhibits and/or blocks and/or prevents nuclear localization of NFIL-3.

In some embodiments of these methods and all such methods described herein, the NFIL-3 inhibitor is an antibody or antigen-binding fragment thereof that binds to the NFIL-3 bound to a target DNA sequence, such as, for example, a sequence selected from any one of SEQ ID NO: 46-SEQ ID NO: 70; such as a sequence at the TIM-3 proximal promoter region and/or intron 1 of the TIM-3 locus and/or a sequence at intron 3 of the TIM-3 locus, and/or a sequence at intron 5 of the TIM-3 locus, but does not bind to either NFIL-3 or the target DNA sequence alone. In some embodiments of these methods and all such methods described herein, the NFIL-3 inhibitor is an antibody or antigen-binding fragment thereof that binds or physically interacts with NFIL-3, and blocks interactions between NFIL-3 and its target DNA sequence, and reduces, impedes, or blocks downstream signaling consequences, such as, for example, IL-10 induction, histone acetylation at a sequence at the TIM-3 locus, TIM-3 mRNA or protein upregulation, and/or elicitation of a cellular response.

In some embodiments of these methods and all such methods described herein, a NFIL-3 inhibitor or antagonist is a monoclonal antibody. In some embodiments of these methods and all such methods described herein, a NFIL-3 inhibitor or antagonist is an antibody fragment or antigen-binding fragment, e.g., as described elsewhere herein.

In some embodiments of these methods and all such methods described herein, an NFIL-3 inhibitor or antagonist is a chimeric antibody derivative of the NFIL-3 antagonist antibodies and antigen-binding fragments thereof. In some embodiments of these methods and all such methods described herein, the NFIL-3 inhibitor or antagonist is a humanized antibody derivative or completely human antibody.

In some embodiments of these methods and all such methods described herein, the NFIL-3 inhibitor is an anti-sense molecule capable of blocking or decreasing the expression of functional NFIL-3 and directed to a nucleic acid encoding NFIL-3 of SEQ ID NO: 5. In some embodiments of these methods and all such methods described herein, the antisense molecules are about 10 to about 100 nucleotides in length, about 15 to about 50 nucleotides in length, about 18 to about 25 nucleotides in length, or more.

In some embodiments of these methods and all such methods described herein, the NFIL-3 inhibitor is a short interfering RNA molecule capable of blocking or decreasing the expression of functional NFIL-3 directed to a nucleic acid encoding acid encoding NFIL-3 of SEQ ID NO: 5.

In some embodiments of these methods and all such methods described herein, the NFIL-3 inhibitor is an RNA or DNA aptamer that binds or physically interacts with NFIL-3, and blocks interactions between NFIL-3 and its target DNA sequence, such as, for example, a sequence selected from any one of SEQ ID NO: 46-SEQ ID NO: 70. In some embodiments of these methods and all such methods described herein, the aptamer comprises at least one RNA or DNA aptamer that binds to the leucine zipper of NFIL-3. In some embodiments of these methods and all such methods described herein, the aptamer comprises at least one RNA or DNA aptamer that binds to any of the phosphorylation sites of NFIL-3.

In some embodiments of these methods and all such methods described herein, the NFIL-3 inhibitor is a small molecule compound or agent that targets or binds to NFIL-3, and/or prevents NFIL-3 binding to promoter regions, such as a sequence at the TIM-3 locus promoter region, and/or prevents NFIL-3-mediated histone acetylation. In some embodiments of these methods and all such methods described herein, a NFIL-3 inhibitor or antagonist comprises a small molecule that selectively binds a target site in the NFIL-3 molecule. In some embodiments of these methods and all such methods described herein, the NFIL-3 inhibitor is a small molecule inhibitor thereof that selectively binds or physically interacts with NFIL-3. In some embodiments of these methods and all such methods described herein, the NFIL-3 inhibitor is a small molecule inhibitor that selectively binds to the leucine zipper domain of NFIL-3 and inhibits and/or blocks and/or prevents binding of NFIL-3 to a target DNA sequence, such as, for example, a sequence selected from any one of SEQ ID NO: 46-SEQ ID NO: 70; such as a sequence at the TIM-3 proximal promoter region and/or a sequence at intron 1 of the TIM-3 locus and/or a sequence at intron 3 of the TIM-3 locus, and/or a sequence at intron 5 of the TIM-3 locus. In some embodiments of these methods and all such methods described herein, the small molecule specifically binds to any of the phosphorylation sites of NFIL-3 and inhibits and/or blocks and/or prevents phosphorylation of NFIL-3. In some embodiments of these methods and all such methods described herein, the small molecule inhibitor binds to NFIL-3 and inhibits and/or blocks and/or prevents nuclear localization of NFIL-3.

In some embodiments of these methods and all such methods described herein, the method further comprises administering any of the IL-27 inhibitors or antagonists described herein.

In regard to the methods of treating chronic immune conditions by decreasing T cell exhaustion and inhibiting TIM-3 activity, immunosuppression of a host immune response plays a role in a variety of chronic immune conditions, such as in persistent infection and tumor immunosuppression. Recent evidence indicates that this immunosuppression can be mediated by immune inhibitory receptors expressed on the surface of an immune cell, and their interactions with their ligands. For example, CD4 T cells can enter a state of “functional exhaustion,” or “unresponsiveness” whereby they express inhibitory receptors that prevent antigen-specific responses, such as proliferation and cytokine production. Accordingly, by inhibiting the activity and/or expression of TIM-3, using IL-27 inhibitors and/or NFIL-3 inhibitors and/or a combination thereof as described herein, an immune response to a persistent infection or to a cancer or tumor that is suppressed, inhibited, or unresponsive, can be enhanced or uninhibited.

As used herein, an “immune response” refers to a response by a cell of the immune system, such as a B cell, T cell (CD4 or CD8), regulatory T cell, antigen-presenting cell, dendritic cell, monocyte, macrophage, NKT cell, NK cell, basophil, eosinophil, or neutrophil, to a stimulus. In some embodiments, the response is specific for a particular antigen (an “antigen-specific response”), and refers to a response by a CD4 T cell, CD8 T cell, or B cell via an antigen-specific receptor. In some embodiments, an immune response is a T cell response, such as a CD4+ response or a CD8+ response. Such responses by these cells can include, for example, cytotoxicity, proliferation, cytokine or chemokine production, trafficking, or phagocytosis, and can be dependent on the nature of the immune cell undergoing the response.

As used herein, “unresponsiveness” or “functional exhaustion” with regard to immune cells includes refractivity of immune cells to stimulation, such as stimulation via an activating receptor or a cytokine. Unresponsiveness can occur, for example, because of exposure to immunosuppressants, exposure to high or constant doses of antigen, or through the activity of inhibitor receptors, such as TIM-3. As used herein, the term “unresponsiveness” includes refractivity to activating receptor-mediated stimulation. Such refractivity is generally antigen-specific and persists after exposure to the antigen has ceased. Unresponsive immune cells can have a reduction of at least 10%, 20%, 30%, 40%, 50%, 60%, 70%, 80%, 90%, 95%, or even 100% in cytotoxic activity, cytokine production, proliferation, trafficking, phagocytotic activity, or any combination thereof, relative to a corresponding control immune cell of the same type.

Accordingly, in some embodiments of the methods of treating chronic immune conditions by decreasing T cell exhaustion and inhibiting TIM-3 activity described herein, the subject being administered the IL-27 or NFIL-3-inhibitor or combination thereof has or has been diagnosed as having a cancer or tumor.

Studies have shown defective or supresssed immune responses in patients diagnosed with cancer. Described herein is the novel finding that absence of IL-27 signaling or NFIL-3 inhibits generation of functionally exhausted T cells or decreases or inhibits functional exhaustion of T cells, and inhibits tumor or cancer growth. Furthermore, described herein is the novel finding that targeting IL-27 signaling or NFIL-3, using, for example, IL-27 or NFIL-3-inhibitor agents as described herein, restores or promotes the responsiveness of these T cells, such that a cancer or tumor is inhibited or reduced.

A “cancer” or “tumor” as used herein refers to an uncontrolled growth of cells which interferes with the normal functioning of the bodily organs and systems. A subject that has a cancer or a tumor is a subject having objectively measurable cancer cells present in the subject's body. Included in this definition are benign and malignant cancers, as well as dormant tumors or micrometastases. Cancers which migrate from their original location and seed vital organs can eventually lead to the death of the subject through the functional deterioration of the affected organs. Hemopoietic cancers, such as leukemia, are able to out-compete the normal hemopoietic compartments in a subject, thereby leading to hemopoietic failure (in the form of anemia, thrombocytopenia and neutropenia) ultimately causing death.

By “metastasis” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

Metastases are most often detected through the sole or combined use of magnetic resonance imaging (MRI) scans, computed tomography (CT) scans, blood and platelet counts, liver function studies, chest X-rays and bone scans in addition to the monitoring of specific symptoms.

Examples of cancer include but are not limited to, carcinoma, lymphoma, blastoma, sarcoma, and leukemia. More particular examples of such cancers include, but are not limited to, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

In some embodiments of these methods and all such methods described herein, the methods further comprise admininstering a tumor or cancer antigen to a subject being administered the IL-27 or NFIL-3-inhibitor agents described herein.

A number of tumor antigens have been identified that are associated with specific cancers. As used herein, the terms “tumor antigen” and “cancer antigen” are used interchangeably to refer to antigens which are differentially expressed by cancer cells and can thereby be exploited in order to target cancer cells. Cancer antigens are antigens which can potentially stimulate apparently tumor-specific immune responses. Some of these antigens are encoded, although not necessarily expressed, by normal cells. These antigens can be characterized as those which are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), and fusion proteins resulting from internal deletions or chromosomal translocations. Still other cancer antigens can be encoded by viral genes such as those carried on RNA and DNA tumor viruses. Many tumor antigens have been defined in terms of multiple solid tumors: MAGE 1, 2, & 3, defined by immunity; MART-1/Melan-A, gp100, carcinoembryonic antigen (CEA), HER-2, mucins (i.e., MUC-1), prostate-specific antigen (PSA), and prostatic acid phosphatase (PAP). In addition, viral proteins such as hepatitis B (HBV), Epstein-Barr (EBV), and human papilloma (HPV) have been shown to be important in the development of hepatocellular carcinoma, lymphoma, and cervical cancer, respectively. However, due to the immunosuppression of patients diagnosed with cancer, the immune systems of these patients often fail to respond to the tumor antigens.

In some embodiments of these methods and all such methods described herein, the methods further comprise admininstering an anti-cancer therapy or agent to a subject in addition to the IL-27 and/or NFIL-3-inhibitor agents described herein.

The term “anti-cancer therapy” refers to a therapy useful in treating cancer. Examples of anti-cancer therapeutic agents include, but are not limited to, e.g., surgery, chemotherapeutic agents, growth inhibitory agents, cytotoxic agents, agents used in radiation therapy, anti-angiogenesis agents, apoptotic agents, anti-tubulin agents, and other agents to treat cancer, such as anti-HER-2 antibodies (e.g., HERCEPTINCD), anti-CD20 antibodies, an epidermal growth factor receptor (EGFR) antagonist (e.g., a tyrosine kinase inhibitor), HER1/EGFR inhibitor (e.g., erlotinib (TARCEVA®)), platelet derived growth factor inhibitors (e.g., GLEEVEC™ (Imatinib Mesylate)), a COX-2 inhibitor (e.g., celecoxib), interferons, cytokines, antagonists (e.g., neutralizing antibodies) that bind to one or more of the following targets ErbB2, ErbB3, ErbB4, PDGFR-beta, BlyS, APRIL, BCMA or VEGF receptor(s), TRAIL/Apo2, and other bioactive and organic chemical agents, etc. Combinations thereof are also specifically contemplated for the methods described herein.

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², P³² es of and radioactive isotopes Lu), chemotherapeutic agents, and toxins such as small molecule toxins or enzymatically active toxins of bacterial, fungal, plant or animal origin, including active fragments and/or variants thereof.

In some embodiments of these methods and all such methods described herein, the methods further comprise admininstering a chemotherapeutic agent to the subject being administered the IL-27 or NFIL-3-inhibitor agents or combination thereof described herein.

Non-limiting examples of chemotherapeutic agents can include include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCINC® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE, vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (TYKERB.); inhibitors of PKC-alpha, Raf, H-Ras, EGFR (e.g., erlotinib (TARCEVA®)) and VEGF-A that reduce cell proliferation and pharmaceutically acceptable salts, acids or derivatives of any of the above. In addition, the methods of treatment can further include the use of radiation or radiation therapy.

As used herein, the terms “chemotherapy” or “chemotherapeutic agent” refer to any chemical agent with therapeutic usefulness in the treatment of diseases characterized by abnormal cell growth. Such diseases include tumors, neoplasms and cancer as well as diseases characterized by hyperplastic growth. Chemotherapeutic agents as used herein encompass both chemical and biological agents. These agents function to inhibit a cellular activity upon which the cancer cell depends for continued survival. Categories of chemotherapeutic agents include alkylating/alkaloid agents, antimetabolites, hormones or hormone analogs, and miscellaneous antineoplastic drugs. Most if not all of these agents are directly toxic to cancer cells and do not require immune stimulation. In one embodiment, a chemotherapeutic agent is an agent of use in treating neoplasms such as solid tumors. In one embodiment, a chemotherapeutic agent is a radioactive molecule. One of skill in the art can readily identify a chemotherapeutic agent of use (e.g. see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2.sup.nd ed., .COPYRGT. 2000 Churchill Livingstone, Inc; Baltzer L, Berkery R (eds): Oncology Pocket Guide to Chemotherapy, 2nd ed. St. Louis, Mosby-Year Book, 1995; Fischer D S, Knobf M F, Durivage H J (eds): The Cancer Chemotherapy Handbook, 4th ed. St. Louis, Mosby-Year Book, 1993).

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one time administration and typical dosages range from 10 to 200 units (Grays) per day.

By “reduce” or “inhibit” in terms of the cancer treatment methods described herein is meant the ability to cause an overall decrease preferably of 20% or greater, 30% or greater, 40% or greater, 45% or greater, more preferably of 50% or greater, of 55% or greater, of 60% or greater, of 65% or greater, of 70% or greater, and most preferably of 75% or greater, 80% or greater, 85% or greater, 90% or greater, or 95% or greater, for a given parameter or symptom. Reduce or inhibit can refer to, for example, the symptoms of the disorder being treated, the presence or size of metastases or micrometastases, the size of the primary tumor, the presence or the size of the dormant tumor, or the load of infectious agent.

In other embodiments of the methods of treating chronic immune conditions by decreasing T cell exhaustion and inhibiting TIM-3 activity described herein, the subject being administered the IL-27 or NFIL-3-inhibitor has or has been diagnosed as having a persistent infection with a bacterium, virus, fungus, or parasite.

“Persistent infections” refer to those infections that, in contrast to acute infections, are not effectively cleared by the induction of a host immune response. During such persistent infections, the infectious agent and the immune response reach equilibrium such that the infected subject remains infectious over a long period of time without necessarily expressing symptoms. Persistent infections often involve stages of both silent and productive infection without rapidly killing or even producing excessive damage of the host cells. Persistent infections include for example, latent, chronic and slow infections. Persistent infection occurs with viruses including, but not limited to, human T-Cell leukemia viruses, Epstein-Barr virus, cytomegalovirus, herpesviruses, varicella-zoster virus, measles, papovaviruses, prions, hepatitis viruses, adenoviruses, parvoviruses and papillomaviruses.

In a “chronic infection,” the infectious agent can be detected in the subject at all times. However, the signs and symptoms of the disease can be present or absent for an extended period of time. Non-limiting examples of chronic infection include hepatitis B (caused by heptatitis B virus (HBV)) and hepatitis C (caused by hepatitis C virus (HCV)) adenovirus, cytomegalovirus, Epstein-Barr virus, herpes simplex virus 1, herpes simplex virus 2, human herpesvirus 6, varicella-zoster virus, hepatitis B virus, hepatitis D virus, papilloma virus, parvovirus B 19, polyomavirus BK, polyomavirus JC, measles virus, rubella virus, human immunodeficiency virus (HIV), human T cell leukemia virus I, and human T cell leukemia virus II. Parasitic persistent infections can arise as a result of infection by, for example, Leishmania, Toxoplasma, Trypanosoma, Plasmodium, Schistosoma, and Encephalitozoon.

In a “latent infection,” the infectious agent (such as a virus) is seemingly inactive and dormant such that the subject does not always exhibit signs or symptoms. In a latent viral infection, the virus remains in equilibrium with the host for long periods of time before symptoms again appear; however, the actual viruses cannot typically be detected until reactivation of the disease occurs. Non-limiting examples of latent infections include infections caused by herpes simplex virus (HSV)-1 (fever blisters), HSV-2 (genital herpes), and varicella zoster virus VZV (chickenpox-shingles).

In a “slow infection,” the infectious agents gradually increase in number over a very long period of time during which no significant signs or symptoms are observed. Non-limiting examples of slow infections include AIDS (caused by HIV-1 and HIV-2), lentiviruses that cause tumors in animals, and prions.

In addition, persistent infections that can be treated using the methods described herein include those infections that often arise as late complications of acute infections. For example, subacute sclerosing panencephalitis (SSPE) can occur following an acute measles infection or regressive encephalitis can occur as a result of a rubella infection.

The mechanisms by which persistent infections are maintained can involve modulation of virus and cellular gene expression and modification of the host immune response. Reactivation of a latent infection can be triggered by various stimuli, including changes in cell physiology, superinfection by another virus, and physical stress or trauma. Host immunosuppression is often associated with reactivation of a number of persistent virus infections.

Additional examples of infectious viruses include: Retroviridae; Picornaviridae (for example, polio viruses, hepatitis A virus; enteroviruses, human coxsackie viruses, rhinoviruses, echoviruses); Calciviridae (such as strains that cause gastroenteritis); Togaviridae (for example, equine encephalitis viruses, rubella viruses); Flaviridae (for example, dengue viruses, encephalitis viruses, yellow fever viruses); Coronaviridae (for example, coronaviruses); Rhabdoviridae (for example, vesicular stomatitis viruses, rabies viruses); Filoviridae (for example, ebola viruses); Paramyxoviridae (for example, parainfluenza viruses, mumps virus, measles virus, respiratory syncytial virus); Orthomyxoviridae (for example, influenza viruses); Bungaviridae (for example, Hantaan viruses, bunga viruses, phleboviruses and Nairo viruses); Arena viridae (hemorrhagic fever viruses); Reoviridae (e.g., reoviruses, orbiviurses and rotaviruses); Birnaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and HSV-2, varicella zoster virus, cytomegalovirus (CMV), herpes viruses); Poxviridae (variola viruses, vaccinia viruses, pox viruses); and Iridoviridae (such as African swine fever virus); and unclassified viruses (for example, the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e., Hepatitis C); Norwalk and related viruses, and astroviruses). The compositions, methods, and uses described herein are contemplated for use in treating infections with these viral agents.

Examples of fungal infections include but are not limited to: aspergillosis; thrush (caused by Candida albicans); cryptococcosis (caused by Cryptococcus); and histoplasmosis. Thus, examples of infectious fungi include, but are not limited to, Cryptococcus neoformans, Histoplasma capsulatum, Coccidioides immitis, Blastomyces dermatitidis, Chlamydia trachomatis, Candida albicans. The compositions, methods, and uses described herein are contemplated for use in treating infections with these fungal agents.

Examples of infectious bacteria include: Helicobacterpyloris, Borelia burgdorferi, Legionella pneumophilia, Mycobacteria sps (such as M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, corynebacterium diphtheriae, corynebacterium sp., Erysipelothrix rhusiopathiae, Clostridium perfringens, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, and Actinomyces israelii. The compositions, methods, and uses described herein are contemplated for use in treating infections with these bacterial agents. Other infectious organisms (such as protists) include: Plasmodium falciparum and Toxoplasma gondii. The compositions, methods, and uses described herein are contemplated for use in treating infections with these agents.

In some embodiments of the aspects described herein, the methods further comprise administering an effective amount of a viral, bacterial, fungal, or parasitic antigen in conjunction with the IL-27 or NFIL-3-inhibitor. Non-limiting examples of suitable viral antigens include: influenza HA, NA, M, NP and NS antigens; HIV p24, pol, gp41 and gp120; Metapneumovirus (hMNV) F and G proteins; Hepatitis C virus (HCV) E1, E2 and core proteins; Dengue virus (DEN1-4) E1, E2 and core proteins; Human Papilloma Virus L1 protein; Epstein Barr Virus gp220/350 and EBNA-3A peptide; Cytomegalovirus (CMV) gB glycoprotein, gH glycoprotein, pp65, IE1 (exon 4) and pp 150; Varicella Zoster virus (VZV) IE62 peptide and glycoprotein E epitopes; Herpes Simplex Virus Glycoprotein D epitopes, among many others. The antigenic polypeptides can correspond to polypeptides of naturally occurring animal or human viral isolates, or can be engineered to incorporate one or more amino acid substitutions as compared to a natural (pathogenic or non-pathogenic) isolate.

In some embodiments, the methods described herein comprise administering an effective amount of the IL-27 or NFIL-3 modulator (i e , inhibitor or activator) described herein to a subject in order to alleviate a symptom of persistent infection. As used herein, “alleviating a symptom of a persistent infection” is ameliorating any condition or symptom associated with the persistent infection. Alternatively, alleviating a symptom of a persistent infection can involve reducing the infectious microbial (such as viral, bacterial, fungal or parasitic) load in the subject relative to such load in an untreated control. As compared with an equivalent untreated control, such reduction or degree of prevention is at least 5%, 10%, 20%, 40%, 50%, 60%, 80%, 90%, 95%, or more as measured by any standard technique. Desirably, the persistent infection is cleared, or pathogen replication has been suppressed, as detected by any standard method known in the art, in which case the persistent infection is considered to have been treated. A patient who is being treated for a persistent infection is one who a medical practitioner has diagnosed as having such a condition. Diagnosis can be by any suitable means. Diagnosis and monitoring can involve, for example, detecting the level of microbial load in a biological sample (for example, a tissue biopsy, blood test, or urine test), detecting the level of a surrogate marker of the microbial infection in a biological sample, detecting symptoms associated with persistent infections, or detecting immune cells involved in the immune response typical of persistent infections (for example, detection of antigen specific T cells that are anergic and/or functionally impaired).

In other aspects, provided herein are methods for the treatment of a chronic immune condition in a subject in need thereof, comprising administering to a subject in need thereof an effective amount of a composition comprising an IL-27 activator or agonist that increases T cell exhaustion by increasing TIM-3 induction and/or activity.

In some embodiments of these methods and all such methods described herein, an IL-27 activator or agonist selectively binds to an IL-27Ra, and increases downstream IL-27Ra signaling, and/or increases or up-regulates IL-27 synthesis, production or release. In some embodiments of these methods and all such methods described herein, an IL-27 activator or agonist increases or enhances expression of IL-27, an IL-27 subunit, or IL-27Ra.

In some embodiments of these methods and all such methods described herein, the IL-27 activator or agonist increases or enhances IL-27 mediated signal transduction. In some embodiments of these methods and all such methods described herein, the IL-27 activator or agonist increases or enhances IL-27 mediated transcription factor induction or activation, for example, e.g., NFIL3 or T-bet induction or activation. In some embodiments of these methods and all such methods described herein, the IL-27 activator or agonist increases or enhances NFIL-3 binding to conserved cis-regulatory regions or sequences at the TIM-3 locus, such as, for example, a sequence selected from any one of SEQ ID NO: 46-SEQ ID NO: 70. In some embodiments of these methods and all such methods described herein, the IL-27 activator or agonist increases or enhances histone acetylation ata sequence at the TIM-3 locus, such as histone acetylation at intron 1. In some embodiments of these methods and all such methods described herein, the IL-27 activator or agonist increases or enhances IL-27 mediated TIM-3 mRNA or protein upregulation. In some embodiments of these methods and all such methods described herein, the IL-27 activator or agonist increases or enhances IL-27-induced IL-10 production.

In some embodiments of these methods and all such methods described herein, the IL-27 activator or agonist is an antibody or antigen-binding fragment thereof that selectively binds or physically interacts with a subunit of IL-27 (IL-27p28 or IL-27Ebi3), and enhances or increases formation of the heterodimeric IL-27. In some embodiments of these methods and all such methods described herein, the binding sites of the IL-27 activator antibody or antigen-binding fragment thereof, are directed against an IL-27R ligand interaction site. In some embodiments of these methods and all such methods described herein, the IL-27 activator or agonist is an antibody or antigen-binding fragment thereof that binds or physically interacts with IL-27Ra. In some embodiments of these methods and all such methods described herein, the IL-27activator or agonist is an antibody or antigen-binding fragment thereof that binds IL-27Ra and increases and/or promotes formation of heterodimeric IL-27 receptor. In some embodiments of these methods and all such methods described herein, the IL-27 activator or agonist is an antibody or antigen-binding fragment thereof that binds IL-27Ra and increase and/or enhances binding between IL-27 and IL-27Ra. In some embodiments of these methods and all such methods described herein, the IL-27 activator or agonist is an antibody or antigen-binding fragment thereof that binds or physically interacts with the heterodimeric IL-27 receptor, and mimics IL-27 binding and increases, upregulates, or enhances, downstream IL-27 signaling, such as, for example, transcription factor induction (e.g., NFIL-3 or T-bet induction), IL-10 induction, histone acetylation at a sequence at the TIM-3 locus, TIM-3 mRNA or protein upregulation, and/or elicitation of a cellular response to IL-27.

In some embodiments of these methods and all such methods described herein, an IL-27 activator or agonist is a monoclonal antibody. In some embodiments of these methods and all such methods described herein, an IL-27 activator or agonist is an antibody fragment or antigen-binding fragment, as described herein above.

In some embodiments of these methods and all such methods described herein, the IL-27 activator or agonist is a small molecule compound or agent. In some embodiments of these methods and all such methods described herein, an IL-27 activator or agonist comprises a small molecule that binds the IL-27R and mimics IL-27 binding. In some embodiments of these methods and all such methods described herein, an IL-27 activator or agonist comprises a small molecule that binds to the IL-27 receptor or to IL-27Ra and increases or promotes an IL-27 biological activity.

In some embodiments of these methods and all such methods described herein, the IL-27 activator or agonist is an RNA or DNA aptamer that binds to the IL-27 receptor and mimics IL-27 binding. In some embodiments of these methods and all such methods described herein, an IL-27 activator or agonist is an RNA or DNA aptamer that binds or physically interacts with IL-27 or the IL-27 receptor, and enhances or promotes interactions between IL-27 and its receptor. In some embodiments of these methods and all such methods described herein, the aptamer comprises at least one RNA or DNA aptamer that binds to the p28 subunit of IL-27. In some embodiments of these methods and all such methods described herein, the aptamer comprises at least one RNA or DNA aptamer that binds to the Ebi3 subunit of IL-27. In some embodiments of these methods and all such methods described herein, an IL-27 activator or agonist comprises at least one RNA or DNA aptamer that binds to both subunits of IL-27. In some embodiments of these methods and all such methods described herein, an IL-27 activator or agonist is an RNA or DNA aptamer that binds or physically interacts with the heterodimeric IL-27 receptor or the IL-27Ra subunit, and increases, enhances, or promotes downstream IL-27 signaling.

In some embodiments of these methods and all such methods described herein, an IL-27 activator or agonist comprises at least one IL-27 structural analog.

In some embodiments of these methods and all such methods described herein, the method further comprises administering any of the NFIL-3 activators or agonists described herein.

Also provided herein, in some aspects, are methods for the treatment of a chronic immune condition in a subject in need thereof, comprising administering to a subject an effective amount of a composition comprising an NFIL-3 activator or agonist that increases T cell exhaustion by increasing TIM-3 induction and/or activity.

In some embodiments of these methods and all such methods described herein, a NFIL-3 activator or agonist increases (activates/enhances) downstream NFIL-3 signaling mediated consequences, such as IL-10 induction, histone acetylation at a sequence at the TIM-3 locus, and/or increases or up-regulates NFIL-3 synthesis, production or release. In some embodiments of these methods and all such methods described herein, an NFIL-3 activator or agonist increases or enhances expression (i.e., transcription or translation) of NFIL-3. In some embodiments of these methods and all such methods described herein, the NFIL-3 activator or agonist increases or enhances NFIL-3 mediated signaling or transcriptional activity. In some embodiments of these methods and all such methods described herein, the NFIL-3 activator or agonist increases or enhances NFIL-3 binding to conserved cis-regulatory regions at the TIM-3 locus, such as, for example, a sequence selected from any one of SEQ ID NO: 46-SEQ ID NO: 70. In some embodiments of these methods and all such methods described herein, the NFIL-3 activator or agonist increases or enhances histone acetylation a sequence at the TIM-3 locus, such as histone acetylation at intron 1. In some embodiments of these methods and all such methods described herein, the NFIL-3 activator or agonist increases or enhances TIM-3 mRNA or protein upregulation. In some embodiments of these methods and all such methods described herein, the NFIL-3 activator or agonist increases or enhances IL-10 production. In some embodiments of these methods and all such methods described herein, the binding sites of the NFIL-3 activators or agonists are directed against a DNA target sequence.

In some embodiments of these methods and all such methods described herein, the NFIL-3 activator or agonist is an antibody or antigen-binding fragment thereof that binds or physically interacts with NFIL-3. In some embodiments of these methods and all such methods described herein, the NFIL-3 activator or agonist is an antibody or antigen-binding fragment thereof that binds NFIL-3 and increases and/or promotes binding of NFIL-3 to a target DNA sequence, such as, for example, a sequence selected from any one of SEQ ID NO: 46-SEQ ID NO: 70. In some embodiments of these methods and all such methods described herein, the NFIL-3 activator or agonist is an antibody or antigen-binding fragment thereof that binds or physically interacts with the NFIL-3 bound to its target DNA sequence, and increases and/or promotes binding and increases, upregulates, or enhances, downstream NFIL-3 signaling consequences, such as, for example, IL-10 induction, histone acetylation ata sequence at the TIM-3 locus, TIM-3 mRNA or protein upregulation, and/or elicitation of a cellular response.

In some embodiments of these methods and all such methods described herein, a NFIL-3 activator or agonist is a monoclonal antibody or an antibody fragment or antigen-binding fragment thereof, as described herein above. In some embodiments of these methods and all such methods described herein, an NFIL-3 activator or agonist is a chimeric antibody derivative of the NFIL-3 agonist antibodies and antigen-binding fragments thereof. In some embodiments of the compositions, methods, and uses described herein, an NFIL-3 activator or agonist is a humanized antibody derivative or completely human antibody or antigen-binding fragments thereof

In some embodiments of these methods and all such methods described herein, the NFIL-3 activator or agonist is a small molecule compound or agent. In some embodiments of these methods and all such methods described herein, an NFIL-3 activator or agonist comprises a small molecule that binds the NFIL-3 target DNA sequence and mimics NFIL-3 binding. In some embodiments of these methods and all such methods described herein, the NFIL-3activator or agonist is a small molecule that selectively binds or physically interacts with NFIL-3. In some embodiments of these methods and all such methods described herein, the NFIL-3 activator or agonist is a small molecule that selectively binds to the leucine zipper domain of NFIL-3 and/or increases or promotes binding of NFIL-3 to a target DNA sequence, such as, for example, a sequence selected from any one of SEQ ID NO: 46-SEQ ID NO: 70; such as a sequence at the TIM-3 proximal promoter region and/or a sequence at intron 1 of the TIM-3 locus and/or a sequence at intron 3 of the TIM-3 locus, and/or a sequence at intron 5 of the TIM-3 locus. In some embodiments of these methods and all such methods described herein, the small molecule activator or agonist specifically phosphorylates any of the phosphorylation sites of NFIL-3. In some embodiments of these methods and all such methods described herein, the small molecule activator or agonist binds to NFIL-3 and increases or promotes nuclear localization of NFIL-3.

In some embodiments of these methods and all such methods described herein, the NFIL-3 activator or agonist is an RNA or DNA aptamer that binds to the NFIL-3 DNA target sequence, such as, for example, a sequence selected from any one of SEQ ID NO: 46-SEQ ID NO: 70, and mimics NFIL-3 binding In some embodiments of these methods and all such methods described herein, a NFIL-3 activator or agonist is an RNA or DNA aptamer that binds or physically interacts with a NFIL-3 DNA target sequence, and enhances or promotes downstream NFIL-3 signaling outcomes by mimicking NFIL-3 binding.

In some embodiments of these methods and all such methods described herein, a NFIL-3 activator or agonist comprises at least one NFIL-3 structural analog.

In some embodiments of these methods and all such methods described herein, the method further comprises administering any of the NFIL-3 activators or agonists described herein.

In some embodiments of the methods of treating chronic immune conditions by increasing T cell exhaustion and increasing TIM-3 induction or activity as described herein, the subject being administered the IL-27 or NFIL-3 activator or agonist or combination thereof has or has been diagnosed with an autoimmune disease or disorder.

As used herein, an “autoimmune disease” refers to a class of diseases in which a subject's own antibodies react with host tissue or in which immune effector T cells are autoreactive to endogenous self-peptides and cause destruction of tissue. Thus an immune response is mounted against a subject's own antigens, referred to as self-antigens. A “self-antigen” as used herein refers to an antigen of a normal host tissue. Normal host tissue does not include cancer cells.

Accordingly, in some embodiments of these methods and all such methods described herein, the autoimmune diseases to be treated or prevented using the methods described herein, include, but are not limited to: rheumatoid arthritis, Crohn's disease or colitis, multiple sclerosis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjogren's syndrome, insulin resistance, and autoimmune diabetes mellitus (type 1 diabetes mellitus; insulin-dependent diabetes mellitus). Autoimmune disease has been recognized also to encompass atherosclerosis and Alzheimer's disease. In some embodiments of the aspects described herein, the autoimmune disease is selected from the group consisting of multiple sclerosis, type-I diabetes, Hashimoto's thyroiditis, Crohn's disease or colitis, rheumatoid arthritis, systemic lupus erythematosus, gastritis, autoimmune hepatitis, hemolytic anemia, autoimmune hemophilia, autoimmune lymphoproliferative syndrome (ALPS), autoimmune uveoretinitis, glomerulonephritis, Guillain-Barre syndrome, psoriasis and myasthenia gravis.

In some embodiments of the methods of treating chronic immune conditions by increasing T cell exhaustion and increasing TIM-3 induction or activityas described herein, the subject being administered the IL-27 or NFIL-3 activator or agonist has or has been diagnosed with host versus graft disease (HVGD). In a further such embodiment, the subject being treated with the methods described herein is an organ or tissue transplant recipient. In other embodiments of the methods of treating chronic immune conditions by increasing T cell exhaustion and increasing TIM-3 induction or activity described herein, the methods are used for increasing transplantation tolerance in a subject. In some such embodiments, the subject is a recipient of an allogenic transplant. The transplant can be any organ or tissue transplant, including but not limited to heart, kidney, liver, skin, pancreas, bone marrow, skin or cartilage. “Transplantation tolerance,” as used herein, refers to a lack of rejection of the donor organ by the recipient's immune system.

The terms “subject” and “individual” as used in regard to any of the methods described herein are used interchangeably herein, and refer to an animal, for example a human, recipient of the bispecific or multispecific polypeptide agents described herein. For treatment of disease states which are specific for a specific animal such as a human subject, the term “subject” refers to that specific animal. The terms “non-human animals” and “non-human mammals” are used interchangeably herein, and include mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like. Production mammal, e.g. cow, sheep, pig, and the like are also encompassed in the term subject.

As used herein, in regard to any of the compositions, methods, and uses comprising IL-27 or NFIL-3 modulators (i e , inhibitors or activators) or combinations thereof described herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder associated with a chronic immune condition, such as, but not limited to, a chronic infection or a cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment).

The term “effective amount” as used herein refers to the amount of an IL-27 or NFIL-3 modulator (i e , inhibitor or activator), or combinations thereof described herein, needed to alleviate at least one or more symptom of the disease or disorder being treated, and relates to a sufficient amount of pharmacological composition to provide the desired effect, i.e., reverse the functional exhaustion of antigen-specific T cells in a subject having a chronic immune condition, such as cancer or hepatitis C. The term “therapeutically effective amount” therefore refers to an amount of the IL-27 or NFIL-3 modulator (i e , inhibitor or activator), or combinations thereof described herein, using the methods as disclosed herein, that is sufficient to provide a particular effect when administered to a typical subject. An effective amount as used herein would also include an amount sufficient to delay the development of a symptom of the disease, alter the course of a symptom disease (for example but not limited to, slow the progression of a symptom of the disease), or reverse a symptom of the disease. Thus, it is not possible to specify the exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions, methods, and uses that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the aIL-27 or NFIL-3 modulator (i e , inhibitor or activator)), or combinations thereof described herein, which achieves a half-maximal inhibition of measured function or activity) as determined in cell culture, or in an appropriate animal model. Levels in plasma can be measured, for example, by high performance liquid chromatography. The effects of any particular dosage can be monitored by a suitable bioassay. The dosage can be determined by a physician and adjusted, as necessary, to suit observed effects of the treatment.

### Modes of Administration

The IL-27 and NFIL-3 modulators (i e , inhibitors and activators), or combinations thereof described herein, described herein can be administered to a subject in need thereof by any appropriate route which results in an effective treatment in the subject. As used herein, the terms “administering,” and “introducing” are used interchangeably and refer to the placement of an IL-27 or NFIL-3 modulator (i e , inhibitor or activator), or a combination thereof, into a subject by a method or route which results in at least partial localization of such agents at a desired site, such as a site of inflammation, such that a desired effect(s) is produced.

In some embodiments, the IL-27 or NFIL-3 modulator (i e , inhibitor or activator) or combination thereof is administered to a subject having a chronic immune condition by any mode of administration that delivers the agent systemically or to a desired surface or target, and can include, but is not limited to, injection, infusion, instillation, and inhalation administration. To the extent that polypeptide agents can be protected from inactivation in the gut, oral administration forms are also contemplated. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments, the IL-27 or NFIL-3 modulators (i e , inhibitors or activators) for use in the methods described herein are administered by intravenous infusion or injection.

The phrases “parenteral administration” and “administered parenterally” as used herein, refer to modes of administration other than enteral and topical administration, usually by injection. The phrases “systemic administration,” “administered systemically”, “peripheral administration” and “administered peripherally” as used herein refer to the administration of the IL-27 and NFIL-3 modulator (i e , inhibitor or activator), or combination thereof, other than directly into a target site, tissue, or organ, such as a tumor site, such that it enters the subject's circulatory system and, thus, is subject to metabolism and other like processes.

For the clinical use of the methods described herein, administration of the IL-27 or NFIL-3 modulators (i e , inhibitors or activators), or combinations thereof described herein, can include formulation into pharmaceutical compositions or pharmaceutical formulations for parenteral administration, e.g., intravenous; mucosal, e.g., intranasal; ocular, or other mode of administration. In some embodiments, the IL-27 or NFIL-3 modulators (i e , inhibitors or activators), or combinations thereof described herein, can be administered along with any pharmaceutically acceptable carrier compound, material, or composition which results in an effective treatment in the subject. Thus, a pharmaceutical formulation for use in the methods described herein can contain an IL-27 or NFIL-3 modulator (i.e., inhibitor or activator), or combination thereof, as described herein in combination with one or more pharmaceutically acceptable ingredients.

The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically acceptable material, composition or vehicle, such as a liquid or solid filler, diluent, excipient, solvent, media, encapsulating material, manufacturing aid (e.g., lubricant, talc magnesium, calcium or zinc stearate, or steric acid), or solvent encapsulating material, involved in maintaining the stability, solubility, or activity of, an IL-27 or NFIL-3 modulator (i.e., inhibitor or activator), or combination thereof. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) excipients, such as cocoa butter and suppository waxes; (8) oils, such as peanut oil, cottonseed oil, safflower oil, sesame oil, olive oil, corn oil and soybean oil; (9) glycols, such as propylene glycol; (10) polyols, such as glycerin, sorbitol, mannitol and polyethylene glycol (PEG); (11) esters, such as ethyl oleate and ethyl laurate; (12) agar; (13) buffering agents, such as magnesium hydroxide and aluminum hydroxide; (14) alginic acid; (15) pyrogen-free water; (16) isotonic saline; (17) Ringer's solution; (19) pH buffered solutions; (20) polyesters, polycarbonates and/or polyanhydrides; (21) bulking agents, such as polypeptides and amino acids (22) serum components, such as serum albumin, HDL and LDL; (23) C2-C12 alchols, such as ethanol; and (24) other non-toxic compatible substances employed in pharmaceutical formulations. Release agents, coating agents, preservatives, and antioxidants can also be present in the formulation. The terms such as “excipient”, “carrier”, “pharmaceutically acceptable carrier” or the like are used interchangeably herein.

The IL-27 or NFIL-3 modulators (i e , inhibitors or activators) or combinations thereof described herein can be specially formulated for administration of the compound to a subject in solid, liquid or gel form, including those adapted for the following: (1) parenteral administration, for example, by subcutaneous, intramuscular, intravenous or epidural injection as, for example, a sterile solution or suspension, or sustained-release formulation; (2) topical application, for example, as a cream, ointment, or a controlled-release patch or spray applied to the skin; (3) intravaginally or intrarectally, for example, as a pessary, cream or foam; (4) ocularly; (5) transdermally; (6) transmucosally; or (79) nasally. Additionally, a bispecific or multispecific polypeptide agent can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 353,270,960.

Further embodiments of the formulations and modes of administration of the compositions comprising IL-27 or NFIL-3 modulators (i.e., inhibitors or activators), or combinations thereof described herein, that can be used in the methods described herein are described below.

Parenteral Dosage Forms. Parenteral dosage forms of the IL-27 or NFIL-3 modulators (i.e., inhibitors or activators), or combinations thereof, can also be administered to a subject with a chronic immune condition by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, controlled-release parenteral dosage forms, and emulsions.

Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Aerosol formulations. An IL-27 or NFIL-3 modulator (i e , inhibitor or activator) or combination thereof can be packaged in a pressurized aerosol container together with suitable propellants, for example, hydrocarbon propellants like propane, butane, or isobutane with conventional adjuvants. An IL-27 or NFIL-3 modulator (i e , inhibitor or activator), or combinations thereof described herein, can also be administered in a non-pressurized form such as in a nebulizer or atomizer. An IL-27 or NFIL-3 modulator (i e , inhibitor or activator), or combinations thereof described herein, can also be administered directly to the airways in the form of a dry powder, for example, by use of an inhaler.

Suitable powder compositions include, by way of illustration, powdered preparations of an IL-27 or NFIL-3 modulator (i.e., inhibitor or activator), or combinations thereof described herein, thoroughly intermixed with lactose, or other inert powders acceptable for intrabronchial administration. The powder compositions can be administered via an aerosol dispenser or encased in a breakable capsule which can be inserted by the subject into a device that punctures the capsule and blows the powder out in a steady stream suitable for inhalation. The compositions can include propellants, surfactants, and co-solvents and can be filled into conventional aerosol containers that are closed by a suitable metering valve.

Aerosols for the delivery to the respiratory tract are known in the art. See for example, Adjei, A. and Garren, J. Pharm. Res., 1: 565-569 (1990); Zanen, P. and Lamm, J.-W. J. Int. J. Pharm., 114: 111-115 (1995); Gonda, I. “Aerosols for delivery of therapeutic and diagnostic agents to the respiratory tract,” in Critical Reviews in Therapeutic Drug Carrier Systems, 6:273-313 (1990); Anderson et al., Am. Rev. Respir. Dis., 140: 1317-1324 (1989)) and have potential for the systemic delivery of peptides and proteins as well (Patton and Platz, Advanced Drug Delivery Reviews, 8:179-196 (1992)); Timsina et. al., Int. J. Pharm., 101: 1-13 (1995); and Tansey, I. P., Spray Technol. Market, 4:26-29 (1994); French, D. L., Edwards, D. A. and Niven, R. W., Aerosol Sci., 27: 769-783 (1996); Visser, J., Powder Technology 58: 1-10 (1989)); Rudt, S. and R. H. Muller, J. Controlled Release, 22: 263-272 (1992); Tabata, Y, and Y. Ikada, Biomed. Mater. Res., 22: 837-858 (1988); Wall, D. A., Drug Delivery, 2: 10 1-20 1995); Patton, J. and Platz, R., Adv. Drug Del. Rev., 8: 179-196 (1992); Bryon, P., Adv. Drug. Del. Rev., 5: 107-132 (1990); Patton, J. S., et al., Controlled Release, 28: 15 79-85 (1994); Damms, B. and Bains, W., Nature Biotechnology (1996); Niven, R. W., et al., Pharm. Res., 12(9); 1343-1349 (1995); and Kobayashi, S., et al., Pharm. Res., 13(1): 80-83 (1996), contents of all of which are herein incorporated by reference in their entirety.

The formulations of the IL-27 or NFIL-3 modulators (i e , inhibitors or activators), or combinations thereof described herein, further encompass anhydrous pharmaceutical compositions and dosage forms comprising the disclosed compounds as active ingredients, since water can facilitate the degradation of some compounds. For example, the addition of water (e.g., 5%) is widely accepted in the pharmaceutical arts as a means of simulating long-term storage in order to determine characteristics such as shelf life or the stability of formulations over time. See, e.g., Jens T. Carstensen, Drug Stability: Principles & Practice, 379-80 (2nd ed., Marcel Dekker, NY, N.Y.: 1995). Anhydrous pharmaceutical compositions and dosage forms of the disclosure can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected. Anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials) with or without desiccants, blister packs, and strip packs.

Controlled and Delayed Release Dosage Forms. In some embodiments of the aspects described herein, an IL-27 or NFIL-3 modulator (i e , inhibitor or activator), or combinations thereof described herein, can be administered to a subject by controlled- or delayed-release means. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Cherng-ju, Controlled Release Dosage Form Design, 2 (Technomic Publishing, Lancaster, Pa.: 2000)). Controlled-release formulations can be used to control a compound of formula (I)'s onset of action, duration of action, plasma levels within the therapeutic window, and peak blood levels. In particular, controlled- or extended-release dosage forms or formulations can be used to ensure that the maximum effectiveness of a compound of formula (I) is achieved while minimizing potential adverse effects and safety concerns, which can occur both from under-dosing a drug (i.e., going below the minimum therapeutic levels) as well as exceeding the toxicity level for the drug.

A variety of known controlled- or extended-release dosage forms, formulations, and devices can be adapted for use with the IL-27 or NFIL-3 modulators (i.e., inhibitors or activators), or combinations thereof described herein. Examples include, but are not limited to, those described in U.S. Pat. Nos.: 3,845,770; 3,916,899; 3,536,809; 3,598,123; 4,008,719; 5674,533; 5,059,595; 5,591 ,767; 5,120,548; 5,073,543; 5,639,476; 5,354,556; 5,733,566; and 6,365,185 B1, each of which is incorporated herein by reference in their entireties. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, DUOLITE® A568 and DUOLITE® AP143 (Rohm&Haas, Spring House, Pa. USA).

In some embodiments of the methods described herein, an IL-27 or NFIL-3 modulator (i.e., inhibitor or activator),or combinations thereof described herein, for use in the methods described herein is administered to a subject by sustained release or in pulses. Pulse therapy is not a form of discontinuous administration of the same amount of a composition over time, but comprises administration of the same dose of the composition at a reduced frequency or administration of reduced doses. Sustained release or pulse administrations are particularly preferred when the disorder occurs continuously in the subject, for example where the subject has continuous or chronic symptoms of a viral infection. Each pulse dose can be reduced and the total amount of an IL-27 or NFIL-3 modulator (i e , inhibitor or activator),or combinations thereof described herein, administered over the course of treatment to the subject or patient is minimized.

The interval between pulses, when necessary, can be determined by one of ordinary skill in the art. Often, the interval between pulses can be calculated by administering another dose of the composition when the composition or the active component of the composition is no longer detectable in the subject prior to delivery of the next pulse. Intervals can also be calculated from the in vivo half-life of the composition. Intervals can be calculated as greater than the in vivo half-life, or 2, 3, 4, 5 and even 10 times greater the composition half-life. Various methods and apparatus for pulsing compositions by infusion or other forms of delivery to the patient are disclosed in U.S. Pat. Nos. 4,747,825; 4,723,958; 4,948,592; 4,965,251 and 5,403,590.

### Further Definitions

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology, and molecular biology can be found in The Merck Manual of Diagnosis and Therapy, 18th Edition, published by Merck Research Laboratories, 2006 (ISBN 0-911910-18-2); Robert S. Porter et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006. Definitions of common terms in molecular biology are found in Benjamin Lewin, Genes IX, published by Jones & Bartlett Publishing, 2007 (ISBN-13: 9780763740634); Kendrew et al. (eds.), The Encyclopedia of Molecular Biology, published by Blackwell Science Ltd., 1994 (ISBN 0-632-02182-9); and Robert A. Meyers (ed.), Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2 ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); or Methods in Enzymology: Guide to Molecular Cloning Techniques Vol.152, S. L. Berger and A. R. Kimmerl Eds., Academic Press Inc., San Diego, USA (1987); Current Protocols in Molecular Biology (CPMB) (Fred M. Ausubel, et al. ed., John Wiley and Sons, Inc.), Current Protocols in Protein Science (CPPS) (John E. Coligan, et. al., ed., John Wiley and Sons, Inc.) and Current Protocols in Immunology (CPI) (John E. Coligan, et. al., ed. John Wiley and Sons, Inc.), which are all incorporated by reference herein in their entireties.

As described herein, an “antigen” is a molecule that is bound by a binding site on a polypeptide agent, such as an antibody. Typically, antigens are bound by antibody ligands and are capable of raising an antibody response in vivo. An antigen can be a polypeptide, protein, nucleic acid or other molecule. In the case of conventional antibodies and fragments thereof, the antibody binding site as defined by the variable loops (L1, L2, L3 and H1, H2, H3) is capable of binding to the antigen. The term “antigenic determinant” refers to an epitope on the antigen recognized by an antigen-binding molecule (such as bispecific polypeptide agent described herein), and more particularly, by the antigen-binding site of said molecule.

As used herein, an “epitope” can be formed both from contiguous amino acids, or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5, about 9, or about 8-10 amino acids in a unique spatial conformation. An “epitope” includes the unit of structure conventionally bound by an immunoglobulin V_{H}/V_{L} pair. Epitopes define the minimum binding site for an antibody, and thus represent the target of specificity of an antibody. In the case of a single domain antibody, an epitope represents the unit of structure bound by a variable domain in isolation. The terms “antigenic determinant” and “epitope” can also be used interchangeably herein.

With respect to a target or antigen, the term “ligand interaction site” on the target or antigen means a site, epitope, antigenic determinant, part, domain or stretch of amino acid residues on the target or antigen that is a site for binding to a ligand, receptor or other binding partner, a catalytic site, a cleavage site, a site for allosteric interaction, a site involved in multimerisation (such as homomerization or heterodimerization) of the target or antigen; or any other site, epitope, antigenic determinant, part, domain or stretch of amino acid residues on the target or antigen that is involved in a biological action or mechanism of the target or antigen, e.g., heterodimeric IL-27, IL27p28, IL-27Ebi3, or NFIL-3. More generally, a “ ligand interaction site” can be any site, epitope, antigenic determinant, part, domain or stretch of amino acid residues on a target or antigen to which a binding site of a bispecific or multispecific polypeptide agent described herein can bind such that the target or antigen (and/or any pathway, interaction, signalling, biological mechanism or biological effect in which the target or antigen is involved) is modulated.

In the context of an antibody or antigen-binding fragment thereof, the term “specificity” or “specific for” refers to the number of different types of antigens or antigenic determinants to which a particular antibody or antigen-binding fragment thereof can bind. The specificity of an antibody or antigen-binding fragment or portion thereof can be determined based on affinity and/or avidity. The affinity, represented by the equilibrium constant for the dissociation (K_{D}) of an antigen with an antigen-binding protein, is a measure for the binding strength between an antigenic determinant and an antigen-binding site on the antigen-binding protein: the lesser the value of the K_{D}, the stronger the binding strength between an antigenic determinant and the antigen-binding molecule. Alternatively, the affinity can also be expressed as the affinity constant (K_{A}), which is 1/K_{D}). As will be clear to the skilled person, affinity can be determined in a manner known per se, depending on the specific antigen of interest. Accordingly, an antibody or antigen-binding fragment thereof as defined herein is said to be “specific for” a first target or antigen compared to a second target or antigen when it binds to the first antigen with an affinity (as described above, and suitably expressed, for example as a K_{D} value) that is at least 10 times, such as at least 100 times, and preferably at least 1000 times, and up to 10.000 times or more better than the affinity with which said amino acid sequence or polypeptide binds to another target or polypeptide. Preferably, when an antibody or antigen-binding fragment thereof is “specific for” a target or antigen, e.g., heterodimeric IL-27, IL27p28, IL-27Ebi3, and/or NFIL-3, compared to another target or antigen, it is directed against said target or antigen, but not directed against such other target or antigen.

Avidity is the measure of the strength of binding between an antigen-binding molecule and the pertinent antigen. Avidity is related to both the affinity between an antigenic determinant and its antigen binding site on the antigen-binding molecule, and the number of pertinent binding sites present on the antigen-binding molecule. Typically, antigen-binding proteins will bind to their cognate or specific antigen with a dissociation constant (K_{D} of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles). Any K_{D} value greater than 10⁻⁴ mol/liter (or any K_{A} value lower than 10⁴ M⁻¹) is generally considered to indicate non-specific binding. The K_{D} for biological interactions which are considered meaningful (e.g., specific) are typically in the range of 10⁻¹⁰ M (0.1 nM) to 10⁻⁵ M (10000 nM). The stronger an interaction is, the lower is its K_{D}. Preferably, a binding site on an IL-27 antagonist antibody or antigen-binding fragment thereof described herein will bind to the desired antigen with an affinity less than 500 nM, preferably less than 200 nM, more preferably less than 10 nM, such as less than 500 pM. Specific binding of an antigen-binding protein to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as other techniques as mentioned herein.

The term “monoclonal antibody” as used herein in regard to any of the IL-27 or NFIL-3 modulating antibodies described herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each antibody in a monoclonal preparation is directed against the same, single determinant on the antigen. It is to be understood that the term “monoclonal antibody” refers to an antibody that is derived from a single clone, including any eukaryotic, prokaryotic, or phage clone, and not the method by which it is produced.The term “monoclonal antibody” as used herein is not limited to antibodies produced through hybridoma technology, and the modifier “monoclonal” is not to be construed as requiring production of the antibody by any particular method. For example, the monoclonal antibodies to be used in accordance with the invention can be made by the hybridoma method first described by Kohler et al., Nature 256:495 (1975), or later adaptations thereof, or can be made by recombinant DNA methods (see, e.g., U.S. Pat. No. 4,816,567). The “monoclonal antibodies”can also be isolated from phage antibody libraries using the techniques described in Clackson et al., Nature 352:624-628 (1991) or Marks et al., J. Mol. Biol. 222:581-597 (1991), for example.

As used herein in regard to any of the IL-27 or NFIL-3 modulating antibodies described herein, the term“chimeric antibody” refers to an antibody molecule in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)). Chimeric antibody molecules can include, for example, one or more antigen binding domains from an antibody of a mouse, rat, or other species, with human constant regions. A variety of approaches for making chimeric antibodies have been described and can be used to make chimeric antibodies containing the immunoglobulin variable region which recognizes the desired antigen, e.g., IL-27 or NFIL-3. See, for example, Takeda et al., 1985, Nature 314:452; Cabilly et al., U.S. Pat. No. 4,816,567; Boss et al.; Tanaguchi et al., European Patent Publication EP171496; European Patent Publication 0173494, United Kingdom patent GB 2177096B).

Humanized forms of non-human (e.g., murine) antibodies are chimeric antibodies which contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies can comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

In some embodiments of the compositions, methods, and uses comprising any of the IL-27 or NFIL-3 modulating antibodies or antigen-binding fragments thereof described herein, the IL-27 or NFIL-3 modulating antibody or antigen-binding fragment is an antibody derivative. For example, but not by way of limitation, antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to specific chemical cleavage, acetylation, formylation, etc. Additionally, the derivative can contain one or more non-classical amino acids.

The IL-27 or NFIL-3 modulating antibodies and antigen-binding fragments thereof described herein (inhibitor/antagonist and/or agonist/activator) for use in modulating T cell exhaustion by modulating TIM-3 induction or activity can be generated by any suitable method known in the art. Monoclonal and polyclonal antibodies against, for example, IL-27, its subunits, and the IL-27 receptor, are known in the art. To the extent necessary, e.g., to generate antibodies with particular characteristics or epitope specificity, the skilled artisan can generate new monoclonal or polyclonal IL-27 antagonist and/or agonist antibodies and/or new monoclonal or polyclonal NFIL-3 antagonist and/or agonist antibodies as briefly discussed herein or as known in the art.

Polyclonal antibodies specific for IL-27, its subunits, the IL-27 receptor, and/or NFIL-3 can be produced by various procedures well known in the art. For example, IL-27 subunit polypeptides or fragments thereof of SEQ ID NO:1, or IL-27 subunit polypeptides or fragments thereof of SEQ ID NO:2, can be administered to various host animals including, but not limited to, rabbits, mice, rats, etc. to induce the production of sera containing polyclonal antibodies specific for the protein. Polyclonal antibodies are preferably raised in animals by multiple subcutaneous (sc) or intraperitoneal (ip) injections of the relevant antigen and an adjuvant. It can be useful to conjugate the antigen to a protein that is immunogenic in the species to be immunized, e.g., keyhole limpet hemocyanin, serum albumin, bovine thyroglobulin, or soy-bean trypsin inhibitor using a bifunctional or derivatizing agent, for example, maleimidobenzoyl sulfosuccinimide ester (conjugation through cysteine residues), N-hydroxy-succinimide (through lysine residues), glutaraldehyde, succinic anhydride, SOCl₂, or R ¹N═C═NR, where R and R¹ are different alkyl groups. Various other adjuvants can be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Suitable adjuvants are also well known to one of skill in the art.

Monoclonal antibodies can be prepared using a wide variety of techniques known in the art including the use of hybridoma, recombinant, and phage display technologies, or a combination thereof Various methods for making monoclonal antibodies described herein are available in the art. For example, the monoclonal antibodies can be made using the hybridoma method first described by Kohler et al., Nature, 256:495 (1975), or any later developments thereof, or by recombinant DNA methods (U.S. Pat. No. 4,816,567). For example, monoclonal antibodies can be produced using hybridoma techniques including those known in the art and taught, for example, in Harlow et al., Antibodies: A Laboratory Manual, (Cold Spring Harbor Laboratory Press, 2nd ed., 1988); Hammer-ling, et al., in: Monoclonal Antibodies and T-Cell Hybridomas 563-681 (Elsevier, N.Y., 1981) (said references incorporated by reference in their entireties). Methods for producing and screening for specific antibodies using hybridoma technology are routine and well known in the art. In another example, antibodies useful in the methods and compositions described herein can also be generated using various phage display methods known in the art, such as isolation from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

Human antibodies can be made by a variety of methods known in the art, including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741, the contents of which are herein incorporated by reference in their entireties.

Human antibodies can also be produced using transgenic mice which express human immunoglobulin genes, and upon immunization are capable of producing a full repertoire of human antibodies in the absence of endogenous immunoglobulin production. For an overview of this technology for producing human antibodies, see, Lonberg and Huszar, 1995, Int. Rev. Immunol. 13:65-93. For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598, the contents of which are herein incorporated by reference in their entireties. In addition, companies such as Abgenix, Inc. (Freemont, Calif.) and Medarex (Princeton, N.J.) can be engaged to provide human antibodies directed against a selected antigen using technology similar to that described above. See also, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and Duchosal et al. Nature 355:258 (1992), the contents of which are herein incorporated by reference in their entireties. Alternatively, phage display technology (McCafferty et al., Nature 348:552-553 (1990)) can be used to produce human antibodies and antibody fragments in vitro, from immunoglobulin variable (V) domain gene repertoires from unimmunized donors. Human antibodies can also be generated by in vitro activated B cells (see U.S. Pat. Nos. 5,567,610 and 5,229,275, the contents of which are herein incorporated by reference in their entireties). Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope (Jespers et al., 1994, Bio/technology 12:899-903).

As used herein, a “blocking” antibody or an antibody “antagonist” is one which inhibits or reduces biological activity of the antigen(s) it binds. For example, an IL-27 antagonist antibody can bind IL-27 and inhibit the ability of IL-27 to, for example, induce NFIL-3 or TIM-3, and/or inhibits the ability of TIM-3 to, for example, bind galectin-9. In certain embodiments, the blocking antibodies or antagonist antibodies or fragments thereof described herein completely inhibit the biological activity of the antigen(s).

“An “Fv” fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the V_{H}-V_{L} dimer. Collectively, the six CDRs or a subset thereof confer antigen binding specificity to the antibody. However, even a single variable domain (or half of an Fv comprising only three CDRs specific for an antigen) has the ability to recognize and bind antigen, although usually at a lower affinity than the entire binding site.

As used herein, “antibody variable domain” refers to the portions of the light and heavy chains of antibody molecules that include amino acid sequences of Complementarity Determining Regions (CDRs; ie., CDR1, CDR2, and CDR3), and Framework Regions (FRs). V_{H} refers to the variable domain of the heavy chain V_{L} refers to the variable domain of the light chain. According to the methods used in this invention, the amino acid positions assigned to CDRs and FRs may be defined according to Kabat (Sequences of Proteins of Immunological Interest (National Institutes of Health, Bethesda, Md., 1987 and 1991)). Amino acid numbering of antibodies or antigen binding fragments is also according to that of Kabat.

As used herein, the term “Complementarity Determining Regions” (CDRs; i.e., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat (i.e. about residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of

Immunological Interest, 5th Ed. Public Health Service, National Institutes of Health, Bethesda, Md. (1991)) and/or those residues from a “hypervariable loop” (i.e. about residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). In some instances, a complementarity determining region can include amino acids from both a CDR region defined according to Kabat and a hypervariable loop. For example, the CDRH1 of the human heavy chain of antibody 4D5 includes amino acids 26 to 35.

“Framework regions” (hereinafter FR) are those variable domain residues other than the CDR residues. Each variable domain typically has four FRs identified as FR1, FR2, FR3 and FR4. If the CDRs are defined according to Kabat, the light chain FR residues are positioned at about residues 1-23 (LCFR1), 35-49 (LCFR2), 57-88 (LCFR3), and 98-107 (LCFR4) and the heavy chain FR residues are positioned about at residues 1-30 (HCFR1), 36-49 (HCFR2), 66-94 (HCFR3), and 103-113 (HCFR4) in the heavy chain residues. If the CDRs comprise amino acid residues from hypervariable loops, the light chain FR residues are positioned about at residues 1-25 (LCFR1), 33-49 (LCFR2), 53-90 (LCFR3), and 97-107 (LCFR4) in the light chain and the heavy chain FR residues are positioned about at residues 1-25 (HCFR1), 33-52 (HCFR2), 56-95 (HCFR3), and 102-113 (HCFR4) in the heavy chain residues. In some instances, when the CDR comprises amino acids from both a CDR as defined by Kabat and those of a hypervariable loop, the FR residues will be adjusted accordingly. For example, when CDRH1 includes amino acids H26-H35, the heavy chain FR1 residues are at positions 1-25 and the FR2 residues are at positions 36-49.

As used herein, a “chimeric antibody” refers to a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science, 1985, 229:1202; Oi et al, 1986, Bio-Techniques 4:214; Gillies et al., 1989, J. Immunol. Methods 125:191-202; U.S. Pat. Nos. 5,807,715; 4,816,567; and 4,816,397, the contents of which are herein incorporated by reference in their entireties.

“Humanized antibodies,” as the term is used herein, refer to antibody molecules from a non-human species, where the antibodies that bind the desired antigen, i.e., IL-27 or NFIL-3, have one or more CDRs from the non-human species, and framework and constant regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., 1988, Nature 332:323. Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology, 1991, 28(4/5):489-498; Studnicka et al., 1994, Protein Engineering 7(6):805-814; Roguska. et al, 1994, PNAS 91:969-973), and chain shuffling (U.S. Pat. No. 5,565,332), the contents of which are herein incorporated by reference in their entireties. Accordingly, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), the contents of which are herein incorporated by reference in their entireties, by substituting rodent CDRs or CDR sequences for the corresponding sequences of a human antibody. Accordingly, such “humanized” antibodies are chimeric antibodies (U.S. Pat. No. 4,816,567, the contents of which are herein incorporated by reference in its entirety) wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The “Fab” fragment contains a variable and constant domain of the light chain and a variable domain and the first constant domain (C_{H}1) of the heavy chain F(ab′)₂ antibody fragments comprise a pair of Fab fragments which are generally covalently linked near their carboxy termini by hinge cysteines between them. Other chemical couplings of antibody fragments are also known in the art.

“Single-chain Fv” or “scFv” antibody fragments comprise the V_{H} and V_{L} domains of antibody, wherein these domains are present in a single polypeptide chain Generally the Fv polypeptide further comprises a polypeptide linker between the V_{H} and V_{L} domains, which enables the scFv to form the desired structure for antigen binding. For a review of scFv, see Pluckthun in The Pharmacology of Monoclonal Antibodies, Vol 113, Rosenburg and Moore eds. Springer-Verlag, New York, pp. 269-315 (1994).

The term “diabodies” refers to small antibody fragments with two antigen-binding sites, which fragments comprise a heavy chain variable domain (V_{H}) connected to a light chain variable domain (V_{L}) in the same polypeptide chain (V_{H} and V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The expression “linear antibodies” refers to the antibodies described in Zapata et al., Protein Eng., 8(10):1057-1062 (1995). Briefly, these antibodies comprise a pair of tandem Fd segments (V_{H} -C_{H}1-V_{H}-C_{H}1) which, together with complementary light chain polypeptides, form a pair of antigen binding regions. Linear antibodies can be bispecific or monospecific.

Various techniques have been developed for the production of antibody or antigen-binding fragments. The antibodies described herein can be fragmented using conventional techniques and the fragments screened for utility in the same manner as described above for the whole antibodies. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 (1985)). For example, Fab and F(ab′)₂ fragments of the bispecific and multispecific antibodies described herein can be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the C_{H}1 domain of the heavy chain However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185.

Examples of techniques which can be used to produce single-chain Fvs and antibodies include those described in U.S. Pat. Nos. 4,946,778 and 5,258,498; Huston et al., 1991, Methods in Enzymology 203:46-88; Shu et al., 1993, PNAS 90:7995-7999; and Skerra et al., 1988, Science 240:1038-1040. For some uses, including the in vivo use of antibodies in humans as described herein and in vitro proliferation or cytotoxicity assays, it is preferable to use chimeric, humanized, or human antibodies.

An “affinity matured” antibody is one with one or more alterations in one or more CDRs thereof which result an improvement in the affinity of the antibody for antigen, compared to a parent antibody which does not possess those alteration(s). Preferred affinity matured antibodies will have nanomolar or even picomolar affinities for the target antigen Affinity matured antibodies are produced by procedures known in the art. Marks et al. Bio/Technology 10:779-783 (1992) describes affinity maturation by V_{H} and V_{L} domain shuffling. Random mutagenesis of CDR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al., J. Mol. Biol. 226:889-896 (1992).

As used herein “complementary” refers to when two immunoglobulin domains belong to families of structures which form cognate pairs or groups or are derived from such families and retain this feature. For example, a V_{H} domain and a V_{L} domain of a natural antibody are complementary; two V_{H} domains are not complementary, and two V_{L} domains are not complementary. Complementary domains can be found in other members of the immunoglobulin superfamily, such as the V_{G}, and Vₚ (or y and 8) domains of the T-cell receptor. Domains which are artificial, such as domains based on protein scaffolds which do not bind epitopes unless engineered to do so, are non-complementary. Likewise, two domains based on, for example, an immunoglobulin domain and a fibronectin domain are not complementary.

The process of designing/selecting and/or preparing a bispecific or multispecific polypeptide agent as described herein, is also referred to herein as “formatting” the amino acid sequence, and an amino acid sequence that is made part of a bispecific or multispecific polypeptide agent described herein is said to be “formatted” or to be “in the format of that bispecific or multispecific polypeptide agent. Examples of ways in which an amino acid sequence can be formatted and examples of such formats will be clear to the skilled person based on the disclosure herein; and such formatted amino acid sequences form a further aspect of the bispecific or multispecific polypeptide agents described herein.

The term “library,” as used herein, refers to a mixture of heterogeneous polypeptides or nucleic acids. The library is composed of members, each of which have a single polypeptide or nucleic acid sequence. To this extent, library is synonymous with repertoire. Sequence differences between library members are responsible for the diversity present in the library. The library can take the form of a simple mixture of polypeptides or nucleic acids, or can be in the form of organisms or cells, for example bacteria, viruses, animal or plant cells and the like, transformed with a library of nucleic acids. Preferably, each individual organism or cell contains only one or a limited number of library members. Advantageously, the nucleic acids are incorporated into expression vectors, in order to allow expression of the polypeptides encoded by the nucleic acids. In a preferred aspect, therefore, a library can take the form of a population of host organisms, each organism containing one or more copies of an expression vector containing a single member of the library in nucleic acid form which can be expressed to produce its corresponding polypeptide member. Thus, the population of host organisms has the potential to encode a large repertoire of genetically diverse polypeptide variants.

Embodiments of the various aspects described herein can be illustrated by the following paragraphs:

A. A method for decreasing T-cell exhaustion in a subject in need thereof, comprising administering to a subject an effective amount of a pharmaceutical composition comprising an IL-27 inhibitor.

B. The method of paragraph A, wherein the IL-27 inhibitor binds IL-27 and inhibits its binding to IL-27R.

C. The method of paragraph A, wherein the IL-27 inhibitor reduces expression of IL-27, an IL-27 subunit, or IL-27Ra.

D. The method of paragraph A, wherein the IL-27 inhibitor decreases IL-27 mediated transcription factor induction or activation.

E. The method of paragraph D, wherein the transcription factor is NFIL-3 (nuclear factor, interleukin-3 regulated).

F. The method of paragraph A, wherein the IL-27 inhibitor decreases NFIL-3 binding to a sequence at the TIM-3 locus.

G. The method of paragraph A, wherein the IL-27 inhibitor decreases histone acetylation at a sequence at the TIM-3 locus.

H. The method of any one of paragraphs F-G, wherein the sequence at the TIM-3 locus comprises a sequence selected from any one of SEQ ID NO: 46-SEQ ID NO: 70.

I. The method of paragraph A, wherein the IL-27 inhibitor decreases TIM-3 mRNA or protein upregulation or expression.

J. The method of any one of paragraphs A-I, wherein the IL-27 inhibitor is an anti-IL-27 antibody or antigen-binding fragment thereof, a small molecule IL-27 inhibitor, an RNA or DNA aptamer that binds or physically interacts with IL-27 or IL-27R, an IL-27 or IL-27 receptor structural analog, a soluble IL-27 receptor, an IL-27 specific antisense molecule, or an IL-27 specific siRNA molecule.

K. A method for decreasing T-cell exhaustion in a subject in need thereof, comprising administering to a subject an effective amount of a pharmaceutical composition comprising an NFIL-3 inhibitor.

L. The method of paragraph K, wherein the NFIL-3 inhibitor binds NFIL-3 and inhibits its binding to a target DNA sequence.

M. The method of paragraph K, wherein the NFIL-3 inhibitor reduces expression of NFIL-3.

N. The method of paragraph K, wherein the NFIL-3 inhibitor decreases NFIL-3 binding to a sequence at the TIM-3 locus

O. The method of paragraph K, wherein the NFIL-3 inhibitor decreases histone acetylation at a sequence at the TIM-3 locus.

P. The method of any one of paragraphs N-0, wherein the sequence at the TIM-3 locus comprises a sequence selected from any one of SEQ ID NO: 46-SEQ ID NO: 70.

Q. The method of paragraph K, wherein the NFIL-3 inhibitor decreases TIM-3 mRNA or protein upregulation or expression.

R. The method of any of paragraphs K-Q, wherein the NFIL-3 inhibitor is an anti-NFIL-3 antibody or antigen-binding fragment thereof, a small molecule NFIL-3 inhibitor, an RNA or DNA aptamer that binds or physically interacts with NFIL-3, an NFIL-3 structural analog, an NFIL-3 specific antisense molecule, or an NFIL-3 specific siRNA molecule.

S. The method of any one of paragraphs A-R, wherein the subject being administered the IL-27 or NFIL-3 inhibitor is diagnosed as having a cancer or tumor.

T. The method of paragraph S, further comprising administering the subject diagnosed as having a cancer or tumor an anti-cancer therapy or agent.

U. The method of any one of paragraphs A-T, wherein the subject being administered the IL-27 or NFIL-3 inhibitor is diagnosed as having a persistent infection.

V. The method of any one of paragraphs A-U, wherein the subject being administered the IL-27 or NFIL-3 inhibitor has a chronic immune condition that comprises a population of functionally exhausted T cells.

W. The method of paragraph V, wherein the population of functionally exhausted T cells comprises a CD4+ T cell population.

X. A method for promoting T cell exhaustion in a subject in need thereof, comprising administering to a subject an effective amount of a pharmaceutical composition comprising an IL-27 activator.

Y. The method of paragraph X, wherein the IL-27 activator binds IL-27 and enhances its binding to IL-27R.

Z. The method of paragraph X, wherein the IL-27 activator increases expression of IL-27, an IL-27 subunit, or IL-27Ra.

AA. The method of paragraph X, wherein the IL-27 activator increases IL-27 mediated transcription factor induction or activation.

BB. The method of paragraph AA, wherein the transcription factor is NFIL-3 (nuclear factor, interleukin-3 regulated).

CC. The method of paragraph X, wherein IL-27 activator increases NFIL-3 binding to a sequence at the TIM-3 locus

DD. The method of paragraph X, wherein the IL-27 activator increases histone acetylation at a sequence at the TIM-3 locus.

EE. The method of any one of paragraphs CC-DD, wherein the sequence at the TIM-3 locus comprises a sequence selected from any one of SEQ ID NO: 46-SEQ ID NO: 70.

FF. The method of paragraph X, wherein the IL-27 activator increases TIM-3 mRNA or protein upregulation or expression.

GG. The method of any one of paragraphs X-FF, wherein the IL-27 activator is an anti-IL-27 antibody or antigen-binding fragment thereof, a small molecule IL-27 activator, an RNA or DNA aptamer that binds or physically interacts with IL-27 or IL-27R, or an IL-27 structural analog.

HH. A method for for promoting T cell exhaustion in a subject in need thereof, comprising administering to a subject an effective amount of a pharmaceutical composition comprising an NFIL-3 activator.

IL The method of paragraph HH, wherein the NFIL-3 activator binds NFIL-3 and enhances its binding to a target DNA sequence.

JJ. The method of paragraph HH, wherein the NFIL-3 activator increases expression of NFIL-3.

KK. The method of paragraph HH, wherein the NFIL-3 activator increases NFIL-3 binding to a sequence at the TIM-3 locus

LL. The method of paragraph HH, wherein the NFIL-3 activator increases histone acetylation at a sequence at the TIM-3 locus.

MM. The method of any one of paragraphs KK-LL, wherein the sequence at the TIM-3 locus comprises a sequence selected from any one of SEQ ID NO: 46-SEQ ID NO: 70.

NN. The method of paragraph MM, wherein the NFIL-3 activator increases TIM-3 mRNA or protein upregulation or expression.

OO. The method of any one of paragraphs HH-NN, wherein the NFIL-3 activator is an anti-NFIL-3 antibody or antigen-binding fragment thereof, a small molecule NFIL-3 activator, an RNA or DNA aptamer that binds or physically interacts with NFIL-3, or an NFIL-3 structural analog.

PP. The method of any one of paragraphs X-00, wherein the subject being administered the IL-27 or NFIL-3 activator is diagnosed as having an autoimmune disorder.

QQ. The method of any one of paragraphs X-00, wherein the subject being administered the IL-27 or NFIL-3 activator is diagnosed as having graft versus host disease or is a transplant recipient.

RR. A pharmaceutical composition comprising an IL-27 inhibitor for use in decreasing T-cell exhaustion.

SS. The use of paragraph RR, wherein the IL-27 inhibitor binds IL-27 and inhibits its binding to IL-27R.

TT. The use of paragraph RR, wherein the IL-27 inhibitor reduces expression of IL-27, an IL-27 subunit, or IL-27Ra.

UU. The use of paragraph RR, wherein the IL-27 inhibitor decreases IL-27 mediated transcription factor induction or activation.

VV. The use of paragraph UU, wherein the transcription factor is NFIL-3 (nuclear factor, interleukin-3 regulated).

WW. The use of paragraph RR, wherein the IL-27 inhibitor decreases NFIL-3 binding to a sequence at the TIM-3 locus.

XX. The use of paragraph RR, wherein the IL-27 inhibitor decreases histone acetylation at a sequence at the TIM-3 locus.

YY. The use of any one of paragraphs WW-XX, wherein the sequence at the TIM-3 locus comprises a sequence selected from any one of SEQ ID NO: 46-SEQ ID NO: 70.

ZZ. The use of paragraph RR, wherein the IL-27 inhibitor decreases TIM-3 mRNA or protein upregulation or expression.

AAA. The use of any one of paragraphs RR-ZZ , wherein the IL-27 inhibitor is an anti-IL-27 antibody or antigen-binding fragment thereof, a small molecule IL-27 inhibitor, an RNA or DNA aptamer that binds or physically interacts with IL-27 or IL-27R, an IL-27 or IL-27 receptor structural analog, a soluble IL-27 receptor, an IL-27 specific antisense molecule, or an IL-27 specific siRNA molecule.

BBB. A pharmaceutical composition comprising an NFIL-3 inhibitor for use in decreasing T-cell exhaustion.

CCC. The use of paragraph BBB, wherein the NFIL-3 inhibitor binds NFIL-3 and inhibits its binding to a target DNA sequence.

DDD. The use of paragraph BBB, wherein the NFIL-3 inhibitor reduces expression of NFIL-3. EEE. The use of paragraph BBB, wherein the NFIL-3 inhibitor decreases NFIL-3 binding to a sequence at the TIM-3 locus

FFF. The use of paragraph BBB, wherein the NFIL-3 inhibitor decreases histone acetylation at a sequence at the TIM-3 locus.

GGG. The use of paragraph EEE-FFF, wherein the sequence at the TIM-3 locus comprises a sequence selected from any one of SEQ ID NO: 46-SEQ ID NO: 70.

HHH. The use of paragraph BBB, wherein the NFIL-3 inhibitor decreases TIM-3 mRNA or protein upregulation or expression.

III. The use of any of paragraphs BBB-HHH, wherein the NFIL-3 inhibitor is an anti-NFIL-3 antibody or antigen-binding fragment thereof, a small molecule NFIL-3 inhibitor, an RNA or DNA aptamer that binds or physically interacts with NFIL-3, an NFIL-3 structural analog, an NFIL-3 specific antisense molecule, or an NFIL-3 specific siRNA molecule.

JJJ. The use of any one of paragraphs RR-III, wherein the T-cell exhaustion is caused or mediated by a cancer or tumor.

KKK. The use of any one of paragraphs RR-III, wherein the T-cell exhaustion is caused or meditated by a persistent infection.

LLL. The use of any one of paragraphs RR-III, wherein the T-cell exhaustion is caused or mediated by a chronic immune condition that comprises a population of functionally exhausted T cells.

MMM. The use of paragraph LLL, wherein the population of functionally exhausted T cells comprises a CD4+ T cell population.

NNN. A pharmaceutical composition comprising an IL-27 activator for use in promoting T cell exhaustion.

OOO. The use of paragraph NNN, wherein the IL-27 activator binds IL-27 and enhances its binding to IL-27R.

PPP. The use of paragraph NNN, wherein the IL-27 activator increases expression of IL-27, an IL-27 subunit, or IL-27Ra.

QQQ. The use of paragraph NNN, wherein the IL-27 activator increases IL-27 mediated transcription factor induction or activation.

RRR. The use of paragraph QQQ, wherein the transcription factor is NFIL-3 (nuclear factor, interleukin-3 regulated).

SSS. The use of paragraph NNN, wherein IL-27 activator increases NFIL-3 binding to a sequence at the TIM-3 locus

TTT. The use of paragraph NNN, wherein the IL-27 activator increases histone acetylation at a sequence at the TIM-3 locus.

UUU. The use of any one of paragraphs SSS-TTT, wherein the sequence at the TIM-3 locus comprises a sequence selected from any one of SEQ ID NO: 46-SEQ ID NO: 70.

VVV. The use of paragraph NNN, wherein the IL-27 activator increases TIM-3 mRNA or protein upregulation or expression.

WWW. The use of any one of paragraphs NNN-VVV, wherein the IL-27 activator is an anti-IL-27 antibody or antigen-binding fragment thereof, a small molecule IL-27 activator, an RNA or DNA aptamer that binds or physically interacts with IL-27 or IL-27R, or an IL-27 structural analog.

XXX. A pharmaceutical composition comprising an NFIL-3 activator for use in promoting T cell exhaustion.

YYY. The use of paragraph XXX, wherein the NFIL-3 activator binds NFIL-3 and enhances its binding to a target DNA sequence.

ZZZ. The use of paragraph XXX, wherein the NFIL-3 activator increases expression of NFIL-3.

AAAA. The use of paragraph XXX, wherein the NFIL-3 activator increases NFIL-3 binding to a sequence at the TIM-3 locus

BBBB. The use of paragraph XXX, wherein the NFIL-3 activator increases histone acetylation at a sequence at the TIM-3 locus.

CCCC. The use of any one of paragraphs AAAA-BBBB, wherein the sequence at the TIM-3 locus comprises a sequence selected from any one of SEQ ID NO: 46-SEQ ID NO: 70.

DDDD. The use of paragraph XXX, wherein the NFIL-3 activator increases TIM-3 mRNA or protein upregulation or expression.

EEEE. The use of paragraph XXX-DDDD, wherein the NFIL-3 activator is an anti-NFIL-3 antibody or antigen-binding fragment thereof, a small molecule NFIL-3 activator, an RNA or DNA aptamer that binds or physically interacts with NFIL-3, or an NFIL-3 structural analog.

FFFF. The use of any one of paragraphs NNN-EEEE, wherein the promotion of T cell exhaustion is for treating an autoimmune disorder.

GGGG. The use of any one of paragraphs NNN-EEEE, wherein the promotion of T cell exhaustion is for treating graft versus host disease or a transplant recipient.

As used herein, the term “comprising” means that other elements can also be present in addition to the defined elements presented. The use of “comprising” indicates inclusion rather than limitation.

As used herein the term “consisting essentially of refers to those elements required for a given embodiment. The term permits the presence of additional elements that do not materially affect the basic and novel or functional characteristic(s) of that embodiment of the invention.

The term “consisting of refers to compositions, methods, and respective components thereof as described herein, which are exclusive of any element not recited in that description of the embodiment.

Further, unless otherwise required by context, singular terms shall include pluralities and plural terms shall include the singular.

Other than in the operating examples, or where otherwise indicated, all numbers expressing quantities of ingredients or reaction conditions used herein should be understood as modified in all instances by the term “about.” The term “about” when used in connection with percentages can mean ±1%.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

All patents and other publications identified are expressly incorporated herein by reference for the purpose of describing and disclosing, for example, the methodologies described in such publications that could be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

This invention is further illustrated by the following examples which should not be construed as limiting.

### EXAMPLES

Tim-3 is an inhibitory receptor the expression of which on effector IFN-y-producing T cells plays an important role in dampening T cell immunity. Sustained Tim-3 expression has been shown to directly result in exhausted/dysregulated phenotype of antigen-specific T cells during chronic viral infections and cancers. As demonstrated herein, IL-27 is a potent inducer of Tim-3 expression. In response to IL-27, transcription factors NFIL3 and T-bet synergistically activate Tim-3 expression. In addition, IL-27 signaling results in profound permissive chromatin remodeling of the Tim-3 locus, favoring Tim-3 transcription. Thus, IL-27 signaling suppresses Type I effector T cell function via induction of Tim-3 expression and other anti-inflammatory molecules including IL-10. Further, IL-27R deficient (WSX-1−/−) mice exhibit significant resistance to tumor growth that is accompanied by a failure to generate Tim-3+ exhausted T cells. The data described herein identify IL-27 as a critical inducer of Tim-3-mediated T cell exhaustion/dysfunction during chronic conditions.

T cell exhaustion is manifested by the progressive loss of function of antigen-specific T cells during chronic viral infections and cancers. Typically, antigen-specific T cells first lose IL-2 production, robust proliferation, and CTL function. Then the cells gradually stop secreting TNF, IFN-γ, and are eventually depleted by apoptosis (1-3).

Inhibitory receptors have key roles in the regulation of T cell exhaustion. PD-1 is the prototypic molecule whose inhibitory function is essential to the induction of T cell exhaustion during chronic LCMV infection in mice and during chronic HIV infection in humans (4-7). As such, PD-1 expression is regarded as a benchmark for exhausted T cells. However, it is now well appreciated that the control of T cell exhaustion exhibits a hierarchical manner. Increased expression of other inhibitory receptors including LAG-3, CD160, CD244 (2B4), and TIM-3 delineates T cells with more deeply exhausted phenotypes (8, 9).

Tim-3 was initially identified as a marker of IFN-y producing CD4+ and CD8+ T cells (10). Interaction between Tim-3 and its ligand galectin-9 suppresses effector T cell function during acute neuroinflammatory disease (11). Multiple studies have demonstrated that Tim-3 is required to maintain the exhausted phenotype of antigen-specific CD4+ and CD8+ T cell in both humans and mice during chronic viral infection such as HIV, HCV, and LCMV (9, 12, 13) and in cancers (14-16). Co-expression of Tim-3 and PD-1 is associated with more severe CD8+ T cell exhaustion. Importantly, while blockade of neither PD-1 nor Tim-3 can effectively reverse T cell exhaustion, simultaneous neutralizing PD-1 and Tim-3 function restores CTL function and cytokine production. These observations indicate that Tim-3 not only serves as a marker for these dysregulated T cells but also functionally cooperate with PD-1 in the regulation of T cell exhaustion (9, 12-16). Thus, targeting Tim-3 as well as other inhibitory receptors on exhausted T cells provides a therapeutic route to treat many chronic conditions.

Although the function of Tim-3 is linked to the suppression of T cell immunity, regulation of its expression in T cells is still under investigation. A previous study showed that Tim-3 is highly induced in terminal differentiated Th1 cells in vitro (10). Only T-bet but not STAT4 seems to be crucial for optimal Tim-3 expression (17), indicating that other cytokine(s) rather than IL-12 maybe more important to induce Tim-3. As demonstrated herein, IL-27 is the most potent cytokine to induce Tim-3 expression on naïve T cells. IL-27 signaling strongly induces the expression of transcription factor NFIL3. NFIL3, in cooperation with T-bet, synergistically induces Tim-3 and IL-10 expression. Importantly, IL-27 stimulation results in permissive chromatin modification in the Tim-3 locus to favor optimal transcription of Tim-3. Such chromatin modification particularly in the promoter and intron 1 regions is highly dependent on the bindings of NFIL3 and T-bet to the Tim-3 locus.

Further, using mouse Bl6F10 myeloma model, IL-27R deficient (WSX-1−/−) mice are demonstrated herein to strongly resist tumor growth concomitantly with significantly reduced exhausted/dysregulated Tim-3+PD-1+CD8+ T cells, and restored pro-inflammatory cytokine production. Thus, the IL-27 pathway is important to suppress T cell immunity via Tim-3 and IL-10 by inducing exhaustion-like dysregulated T cells. Such exhaustion-like effect is at least partially dependent on the function of NFIL3. Taken together, the results described herein provide novel therapeutic routes to treat chronic conditions by targeting the IL-27-Tim-3 pathway.

### IL-27 is a Potent Inducer of Tim-3 and IL-10 Expression in Naïve CD4+ T Cells.

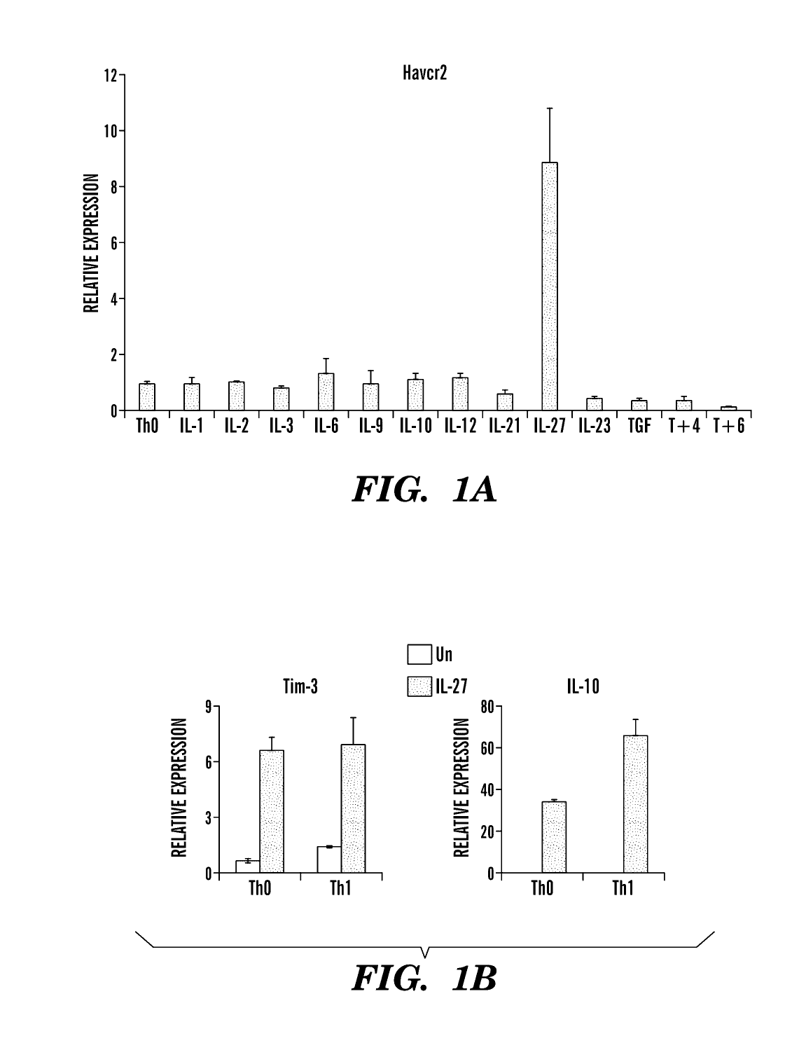

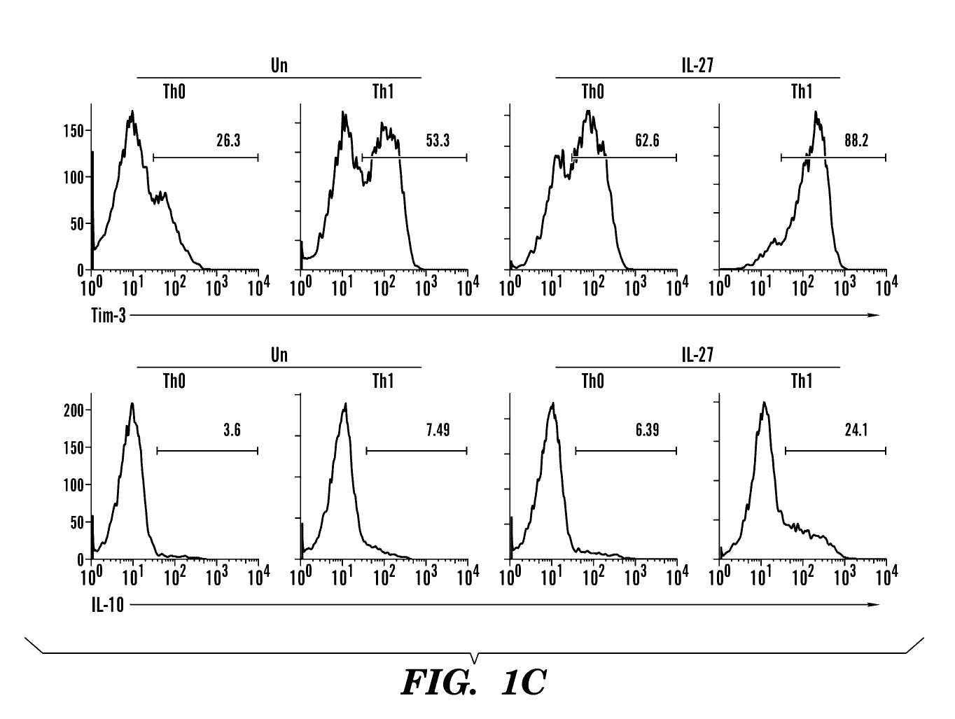

A previous study showed that Tim-3 expression on Thl cells is T-bet, but not STAT4, dependent (17), indicating that Tim-3 expression is not totally dependent on IL-12 signaling. To further explore other Tim-3 inducer(s), naïve CD4+ T cells were activated by anti-CD3 and anti-CD28 antibodies in the presence of a panel of cytokines. After analyzing Tim-3 transcription by real time PCR, it was found that IL-27 is the most potent inducer of Tim-3 expression (FIG. 1A). Interestingly, compared with neutral condition (Th0), IL-12 only slightly increases Tim-3 transcription (FIG. 1A).

Since Tim-3 expression is associated with IFN-y-secreting T cells (10), whether IL-27 could further enhance Tim-3 expression on Thl cells was determined. Th0 and Thl cells were treated with IL-27 during TcR activation and it was found that IL-27 is a dominant signal molecule to induce Tim-3 transcription. By contrast, IL-12 only has a minor effect on Tim-3 transcription in naïve CD4+ T cells (FIG. 1B). IL-27 and IL-12 together do not synergistically increase Tim-3 transcription (FIG. 1B). The regulation of Tim-3 protein expression by IL-27 and IL-12 minors the trend in the transcriptional level. Combination of IL-27 and IL-12 exhibited an additive effect to drive Tim-3 protein expression (FIG. 1C). IL-10 induction is a key mechanism of IL-27-mediated anti-inflammatory effect (18, 19). IL-10 expression in Th0 and Thl cells was examined with or without the presence of IL-27. Similar to its role in the induction of Tim-3 expression, IL-27 is a dominant cytokine to induce IL-10. However, there is a strong synergy between IL-27 and IL-12 in the induction of IL-10 expression on both transcriptional and translational levels (FIGS. 1B-1C).

### Transcription Factor NFIL3 is Required for Tim-3 Expression.

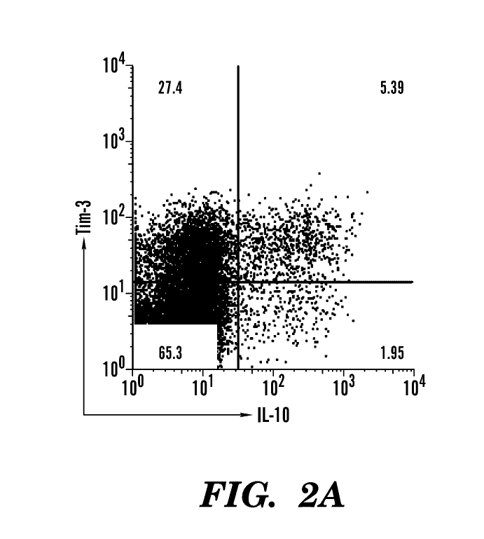

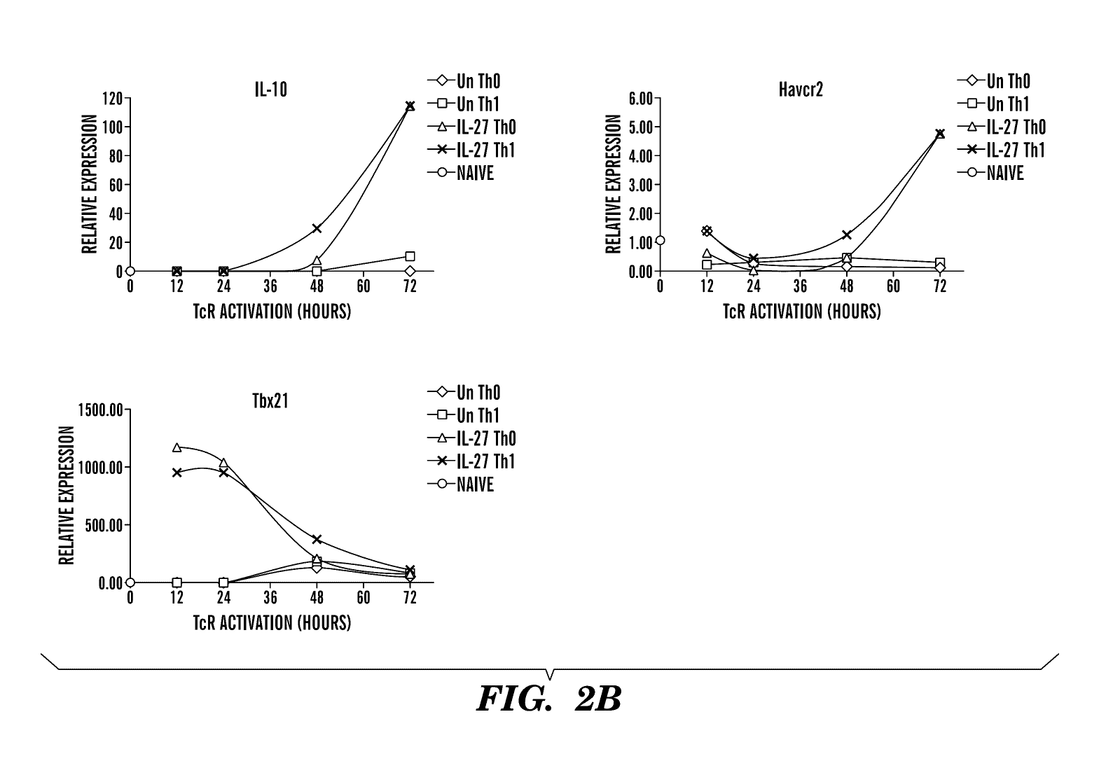

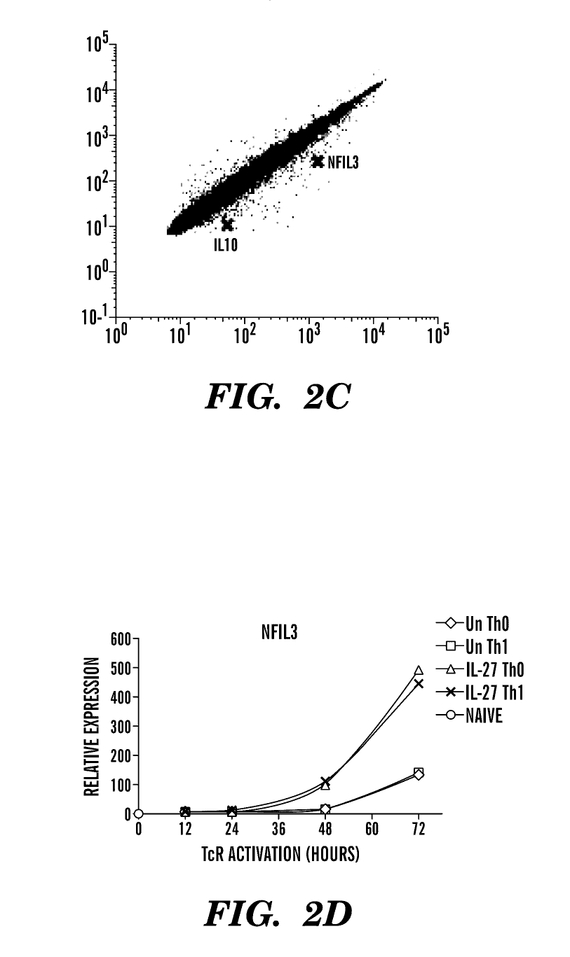

As an inhibitory receptor, Tim-3 can serve as an important effector molecule in IL-27-mediated suppression of Thl/Tcl immunity. When analyzing Tim-3 and IL-10 expression on IL-27-treated Thl cells, it was found that the majority of IL-10 producing cells were Tim-3 positive (FIG. 2A). To understand the kinetics of Tim-3 and IL-10 expression during IL-27 stimulation, naïve Th0 and Th1 cells were activated with or without the presence of IL-27 and RNA harvested at different time points for TAQMAN PCR analysis. Tim-3 and IL-10 transcription exhibited a similar kinetics (FIG. 2B). Interestingly, T-bet is a transcription factor required for both Tim-3 and IL-10 expression (17, 20). It was tested whether a largely overlapped transcription network regulates the expression of Tim-3 and IL-10.

IL-27-induced T-bet mRNA level peaks within 24 hours after T cell activation. In contrast, Tim-3 and IL-10 transcription lags behind T-bet mRNA expression (FIG. 2B). Interestingly, T-bet transcription actually declines when Tim-3 and IL-10 transcription begins to increase, indicating that T-bet induction is required for Tim-3 and IL-10 expression probably as a priming factor. Other transcription factors are necessary for further increased transcription of Tim-3 and IL-10.

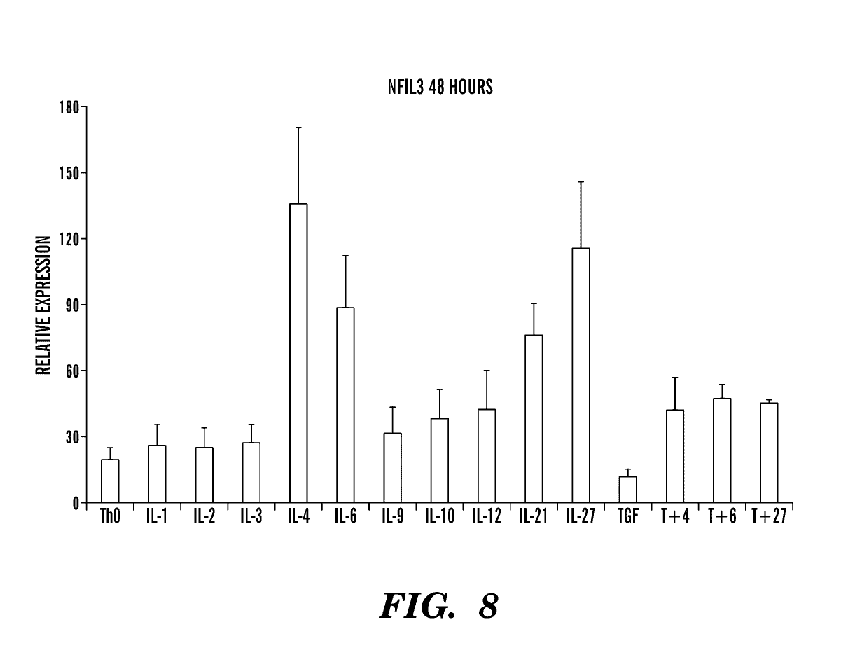

To understand IL-27-mediated transcription factor network, a gene profile study was conducted on IL-27-stimulated naïve CD4+ T cells by which microarray analysis was performed using RNA harvested 60 hours after IL-27 stimulation. Among a list of IL-27-induced transcription factors, transcription factors that are involved in IL-10 expression were studied. Nuclear factor, interleukin 3 regulated (NFIL3) is a transcription factor whose expression was recently found upregulated in terminal differentiated Th1 cells and essential for their expression of IL-10 (21). We demonstrated for the first time that transcription of NFIL3 is highly induced by IL-27 (FIG. 2C, FIG. 8). Interestingly, its transcription kinetics closely parallel those of IL-10 and Tim-3 (FIGS. 2B and 2D), thereby making it an appealing candidate for the regulation of Tim-3 transcription.

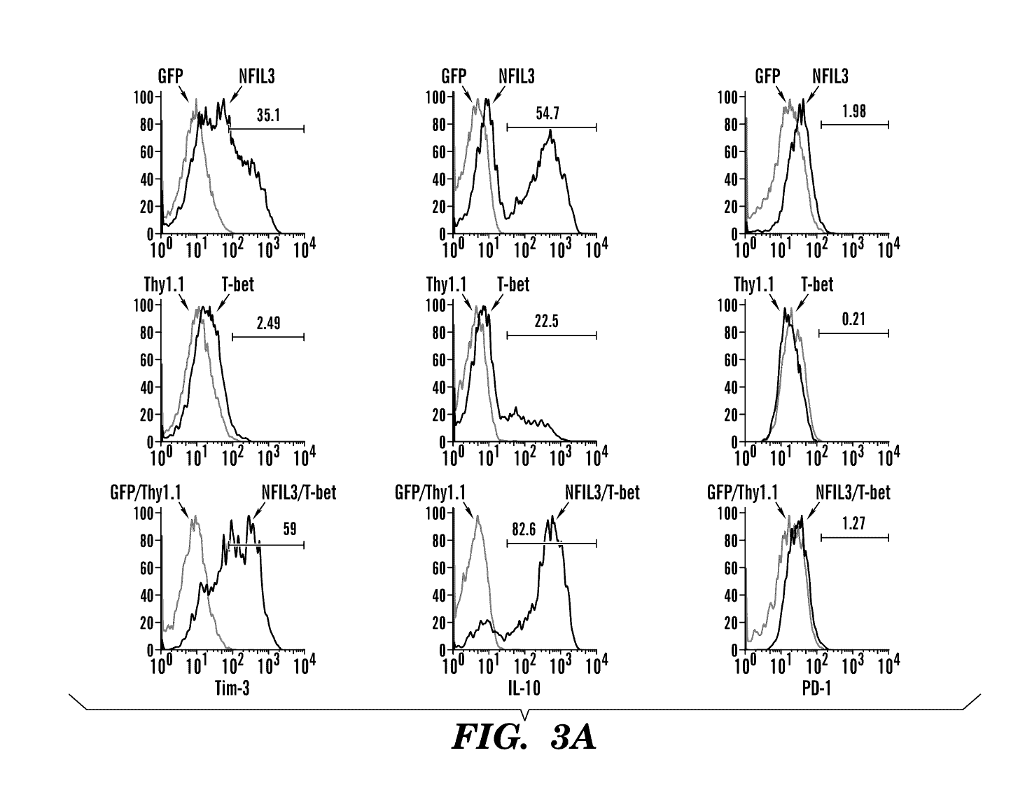

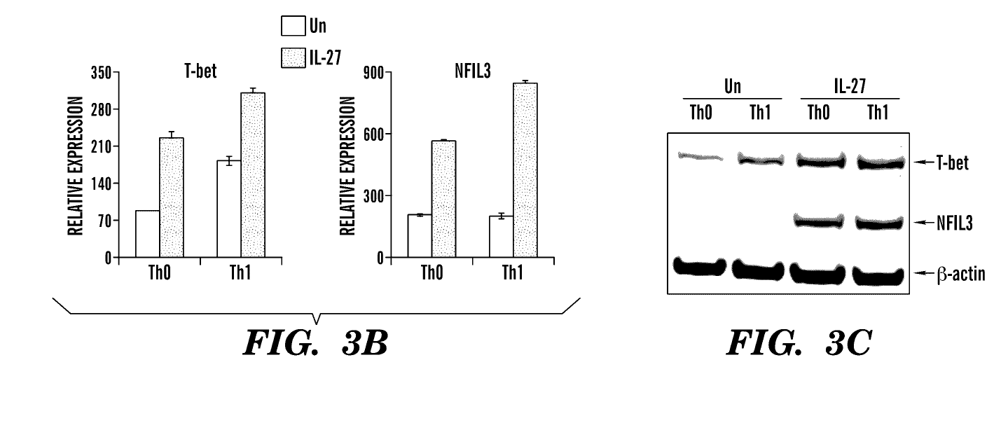

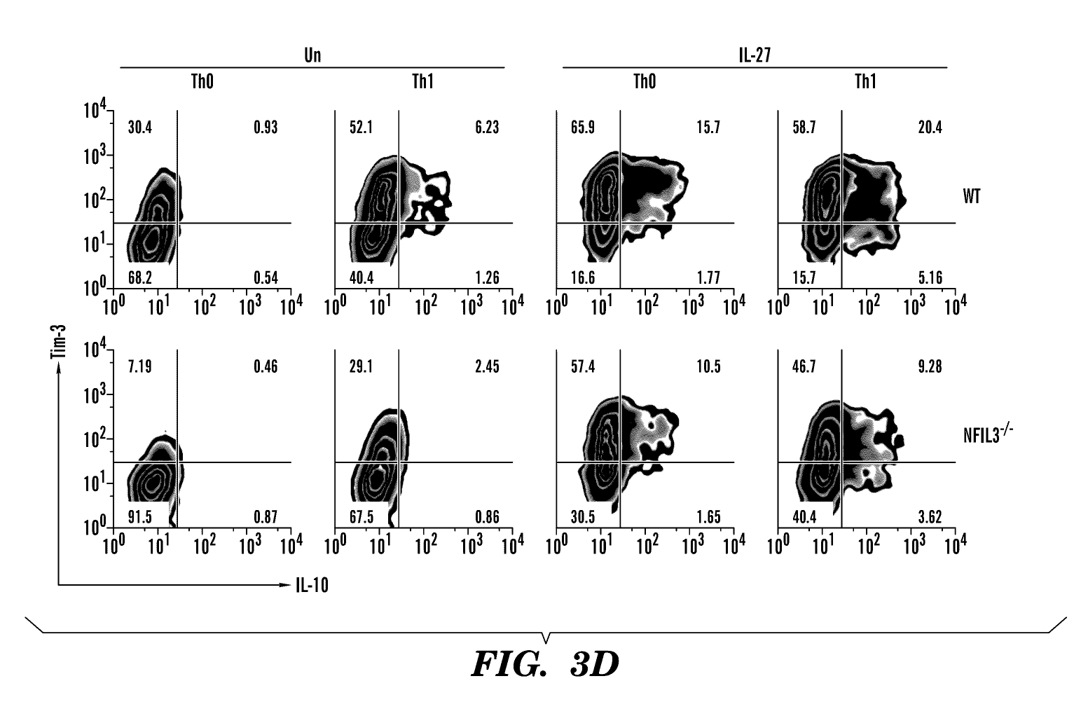

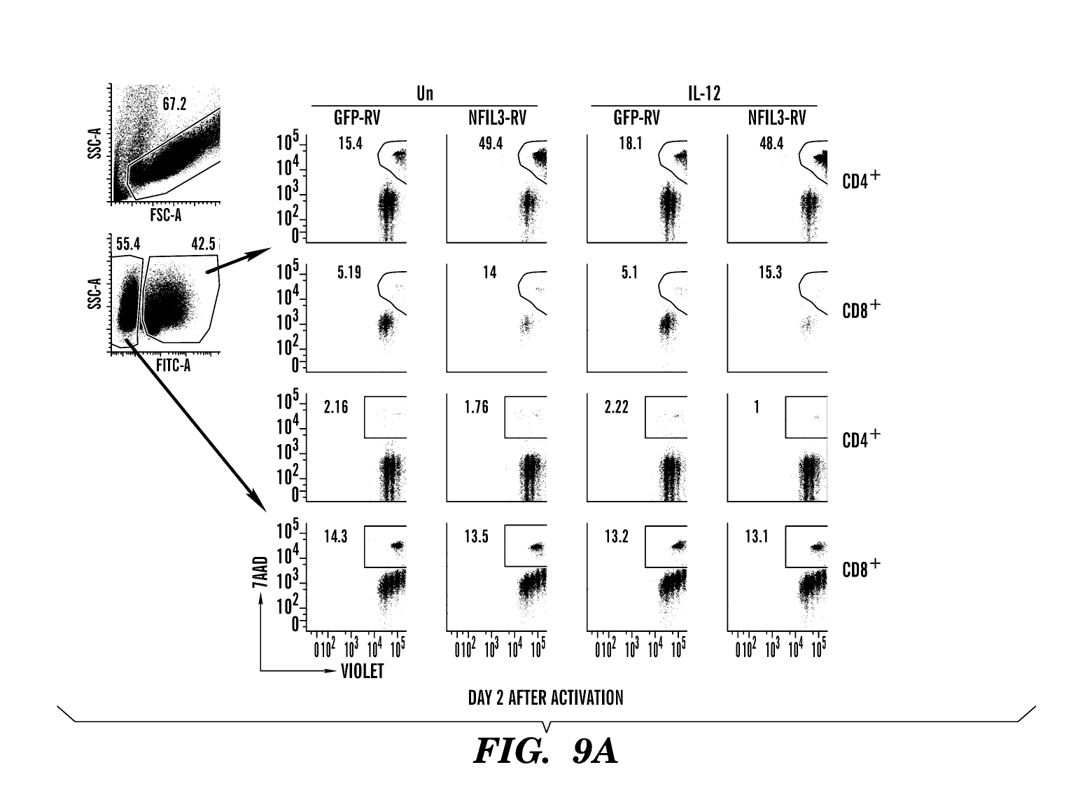

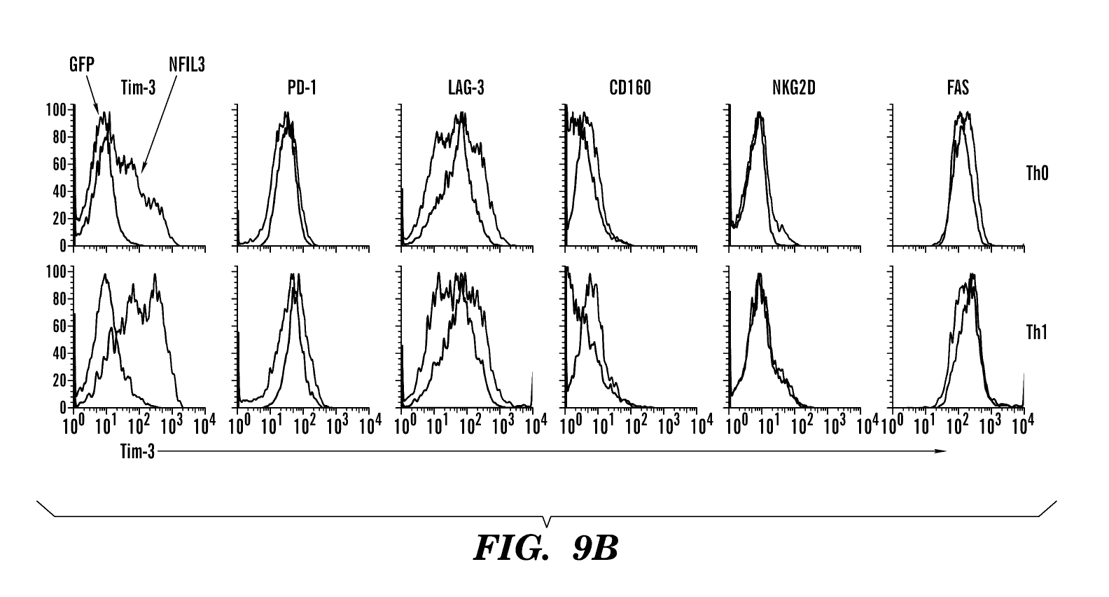

To understand the role of NFIL3 in the regulation of T cell activity, a retroviral expression construct for NFIL3 (NFIL3) was generated and ectopically expressed NFIL3 in naïve CD4+ T cells. Compared with the cells that were only transduced with empty retroviral vector (GFP), the proliferation of NFIL3-transduced T cells was not affected. However, overexpression of NFIL3 significantly increased cell death (FIG. 9A). Next, cell surface staining was conducted and it found that forced expression of NFIL3 dramatically increased Tim-3 expression (FIG. 3A). This effect is highly specific for Tim-3, since NFIL3 only slightly increased the expression of other inhibitory receptors such as PD-1, LAG3, and CD160 (FIGS. 3A, 9B). Interestingly, NFIL3-transduced Th1 cells expressed higher level of Tim-3 than NFIL3-transduced Th0 cells (FIG. 9B), indicating a potentially functional cooperation between NFIL3 and the IL-12 pathway in the expression of Tim-3. Cytokine production was also checked and it was found that NFIL3-transduced cells significantly increased IL-10 expression (FIG. 3A). Taken together, these data support that NFIL3 is a transcription factor to drive Tim-3 and IL-10 expression thereby suppressing T cell activation.

### NFIL3 and T-Bet Synergistically Activate Tim-3 and IL-10 Expression

The preferential induction of Tim-3 expression but not other inhibitory receptors by NFIL3 indicates that NFIL3 is a target specific transcription factor. Since previous work had identified that T-bet is required for Tim-3 expression (17), the potential functional cooperation between NFIL3 and T-bet in the regulation of Tim-3 and IL-10 expression in Th0 cells, where endogenous T-bet and NFIL3 are both low, was examined. After ectopically expressing NFIL3 or T-bet using retroviral vector (NFIL3 or T-bet) in naïve CD4+ T cell, it was found that Tim-3 expression was enhanced by either NFIL3 or T-bet. Interestingly, overexpression of NFIL3 seems more potent to induce both Tim-3 and IL-10 expression (FIG. 3A). Further, ectopic expression of both NFIL3 and T-bet synergistically enhanced Tim-3 expression. Similar effect was also observed in the regulation of IL-10 expression. Interestingly, NFIL3 and T-bet exhibited opposite roles in the regulation of PD-1 expression. While NFIL3 slightly enhanced the expression of PD-1, overexpression of T-bet actually suppressed its expression (FIG. 3A). Such inhibitory effect of T-bet on PD-1 expression mirrors other's observation (22). Thus, fundamental difference in transcriptional regulation of PD-1 and Tim-3 expression was demonstrated.

Next, the expression of T-bet and NFIL3 in T cells in response to IL-27 and IL-12 stimulation was examined. Both transcription factors exhibited low expression in Th0 cells. While a slight induction of T-bet was found in IL-12-treated cells, there was no significant change in NFIL3 expression by IL-12 treatment. However, both T-bet and NFIL3 were strongly induced by IL-27. Such upregulation was further enhanced by the presence of IL-12 (FIGS. 3B-3C). Thus, IL-27-mediated Tim-3 and IL-10 expression is largely dependent on proportionally induced T-bet and NFIL3.

### NFIL3−/− CD4+ T Cells Express Less Amounts of Tim-3 and IL-10

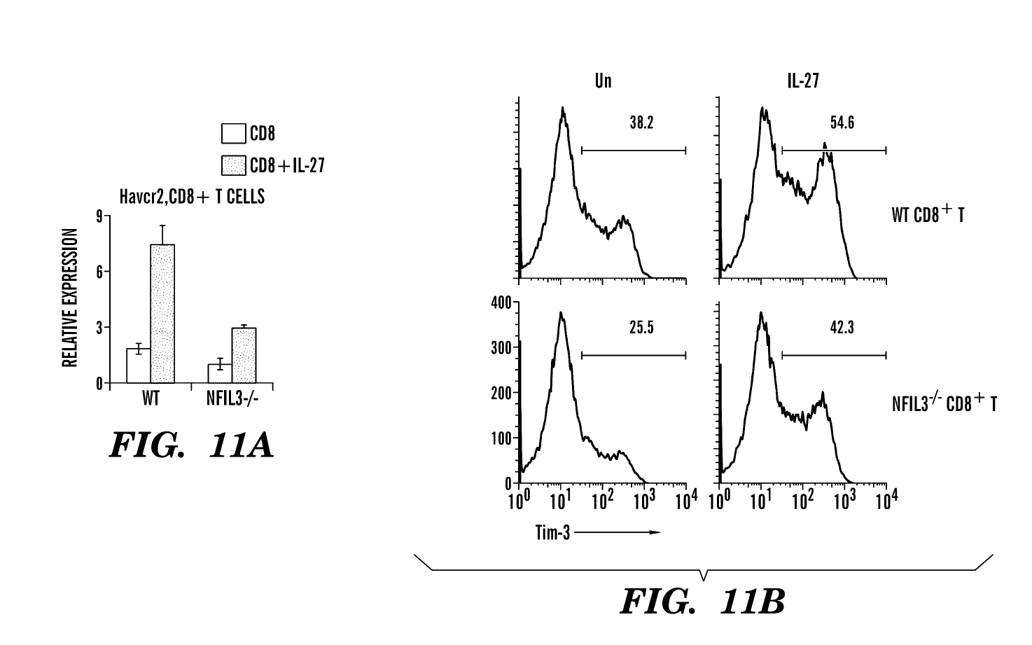

To confirm the role of NFIL3 in the regulation of Tim-3 and IL-10 expression, naïve wild type (WT) and NFIL3−/− CD4+ T cells were cultured in vitro under Th0 or Th1 condition with or without the presence of IL-27. It was found that NFIL3 deficiency resulted in reduced Tim-3 and IL-10 expression under all of the culture conditions, indicating that NFIL3 is critical for the expression of both Tim-3 and IL-10 (FIG. 3D). The robust induction of Tim-3 and IL-10 expression by IL-27 is likely dependent on its ability to induce a high amount of NFIL3 expression. Likewise, NFIL3 deficiency in CD8+ T cells led to significant reduction of Tim-3 expression when cells were cultured under either neutral condition (Tc) or the presence of IL-27 (FIGS. 11A, 11B).

IL-27-Mediated Tim-3 and IL-10 Expression is Dependent on both STAT1 and STAT3 Pathways

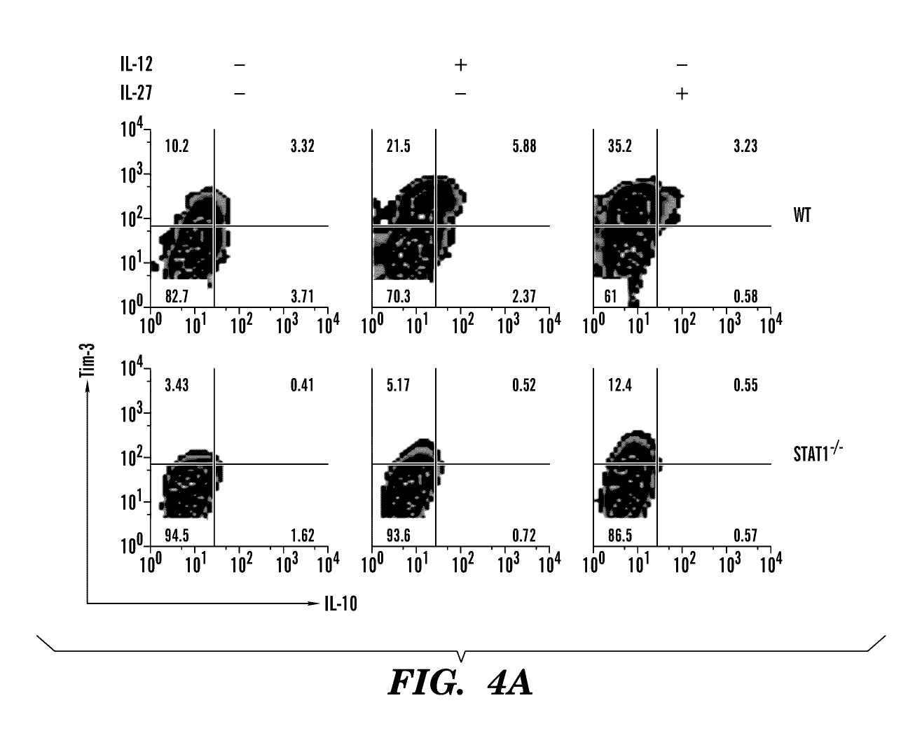

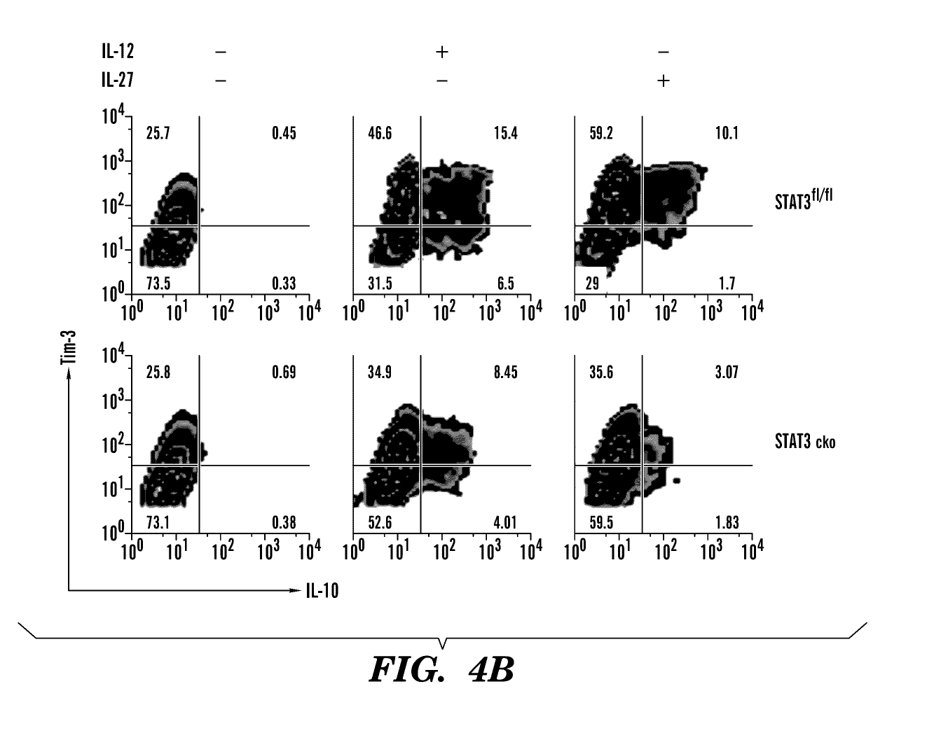

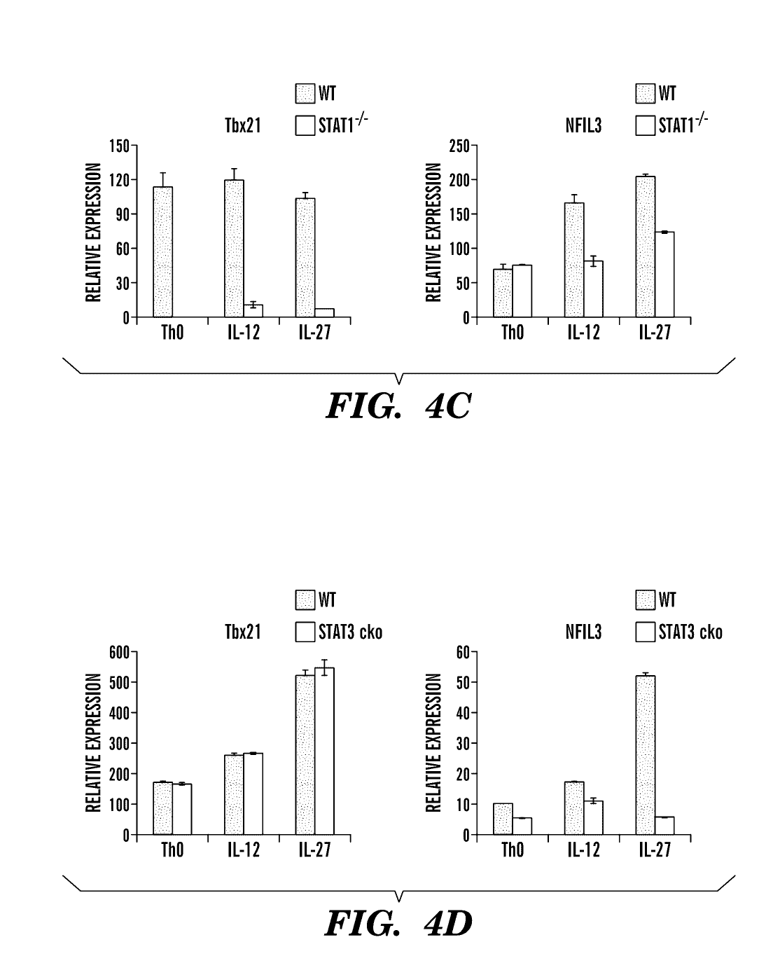

STAT1 and STAT3 are two major transducers in the IL-27 signaling pathway. To study their role of in Tim-3 and IL-10 expression, naïve CD4+ T cells from STAT1−/− and STAT3fl/fl×CD4-Cre (STAT3 cko) mice were activated in the presence of either IL-27 or IL-12. Through flow cytometry analysis, it was found that STAT1 deficient Th0 cells significantly attenuated the expression of Tim-3 and IL-10 compared with WT Th0 cells. Such defect became more pronounced when STAT1 was stimulated by IL-27 or IL-12 (FIG. 4A). Interestingly, STAT3 deficiency does not appear to affect Tim-3 or IL-10 expression in cells cultured under neutral condition. But IL-27-treated STAT3 deficient CD4+ T cells clearly exhibited reduced Tim-3 and IL-10 expression compared to WT cells. Surprisingly, the expressional defect was also found in IL-12 treated cells, indicating an important role of STAT3 in IL-12-mediated Tim-3 and IL-10 expression (FIG. 4B).

T-bet and NFIL3 transcription level in STAT1−/− or STAT3 cko CD4+ T cells was further analyzed. The absence of STAT1, but not STAT3, resulted in profound reduction of T-bet transcription either in neutral culture condition or in the presence of IL-12 or IL-27, indicating that STAT1, but not STAT3, is essential for T-bet expression (FIGS. 4C-4D). By contrast, STAT3 seems to be a dominant factor that controls NFIL3 transcription. STAT3 deficiency exhibited reduced NFIL3 transcription under neutral culture condition, indicating that STAT3 is essential for NFIL3 expression. STAT3 deficient CD4+ T cells completely lost IL-27-induced NFIL3 expression (FIG. 4D). Although STAT1 deficiency also resulted in a slight reduction of NFIL3 mRNA level in IL-12- and IL-27-stimuated cells, such reduction was not found in the cells under Th0 conditions, indicating that STAT1 also mediates NFIL3 expression. However, STAT1 probably is not major pathway to induce NFIL3 expression, therefore the optimal NFIL3 expression in Thl cells happens after several rounds of in vitro polarization (FIGS. 4C-4D). As such, IL-27 stimulation results in activation of two distinct and non-redundant pathways that are controlled by STAT1/T-bet and STAT3/NFIL3. They are both critical for the induction of Tim-3, and IL-10, expression in T cells.

### IL-27 Directly Induces Tim-3 Expression via NFIL3 and T-Bet.

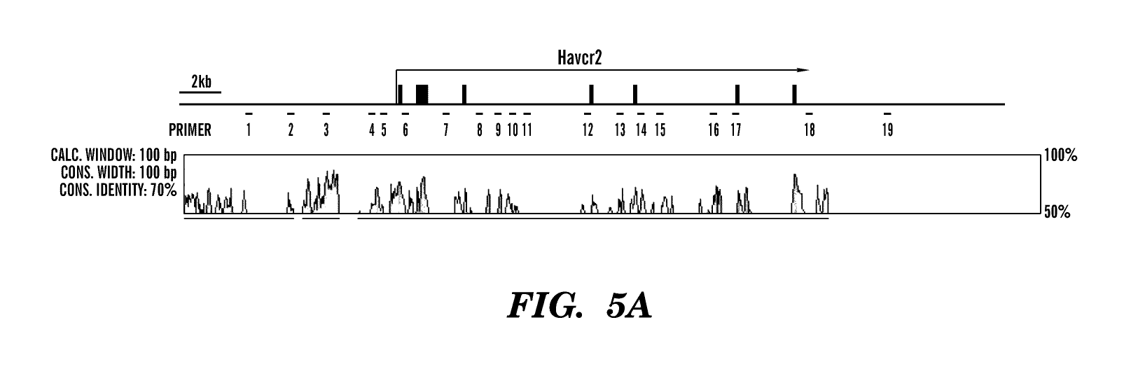

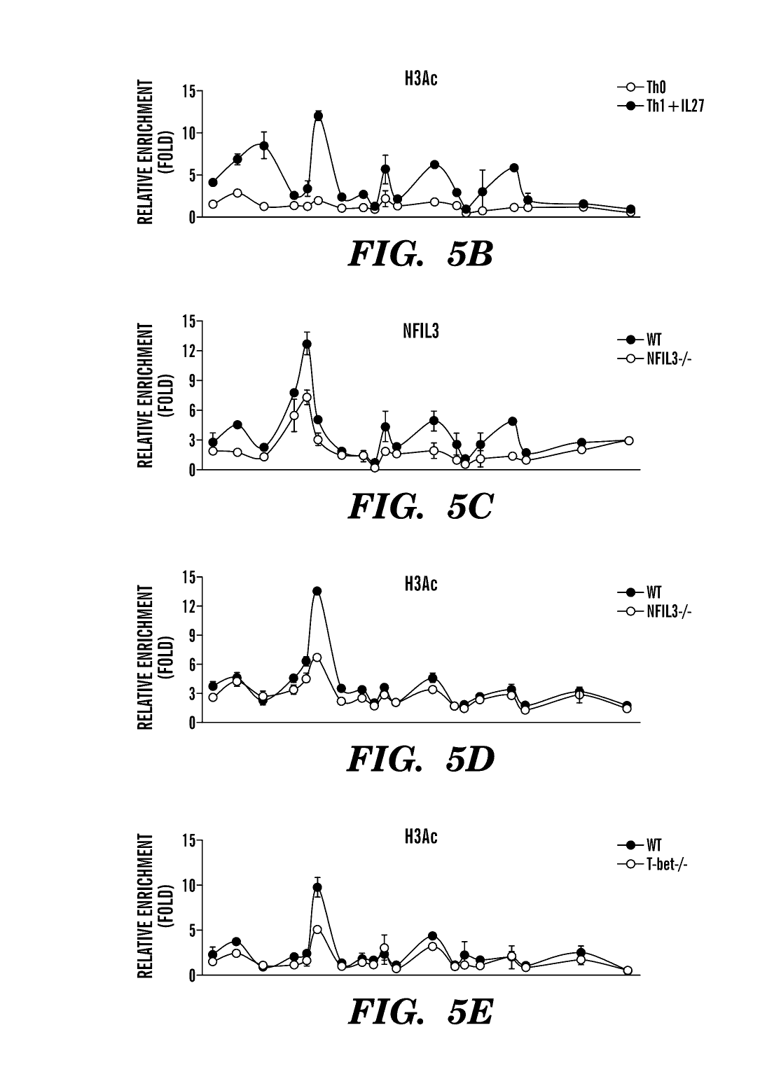

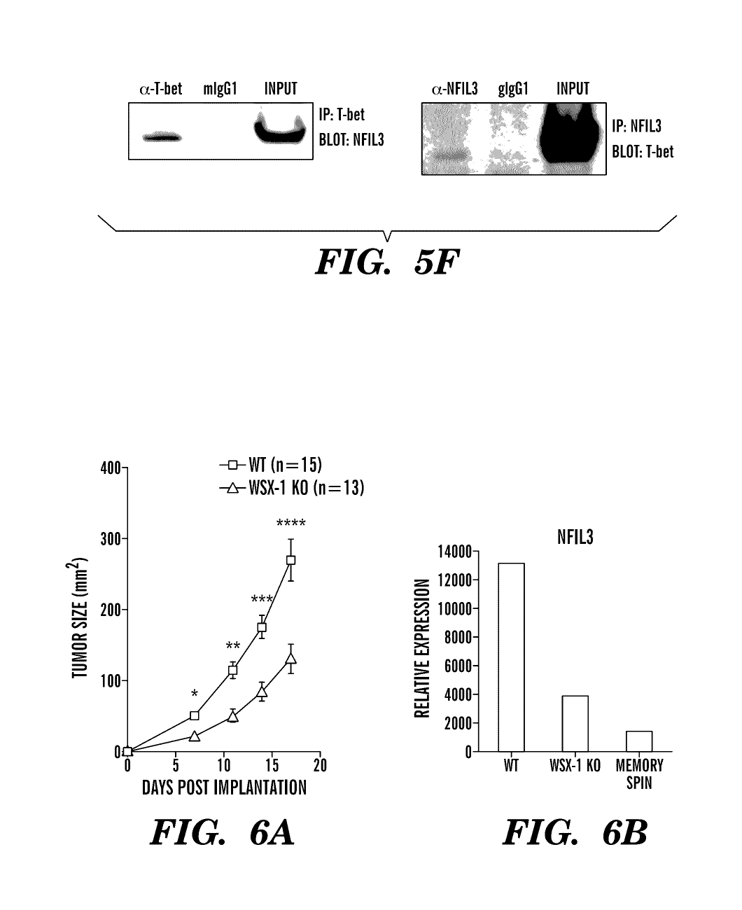

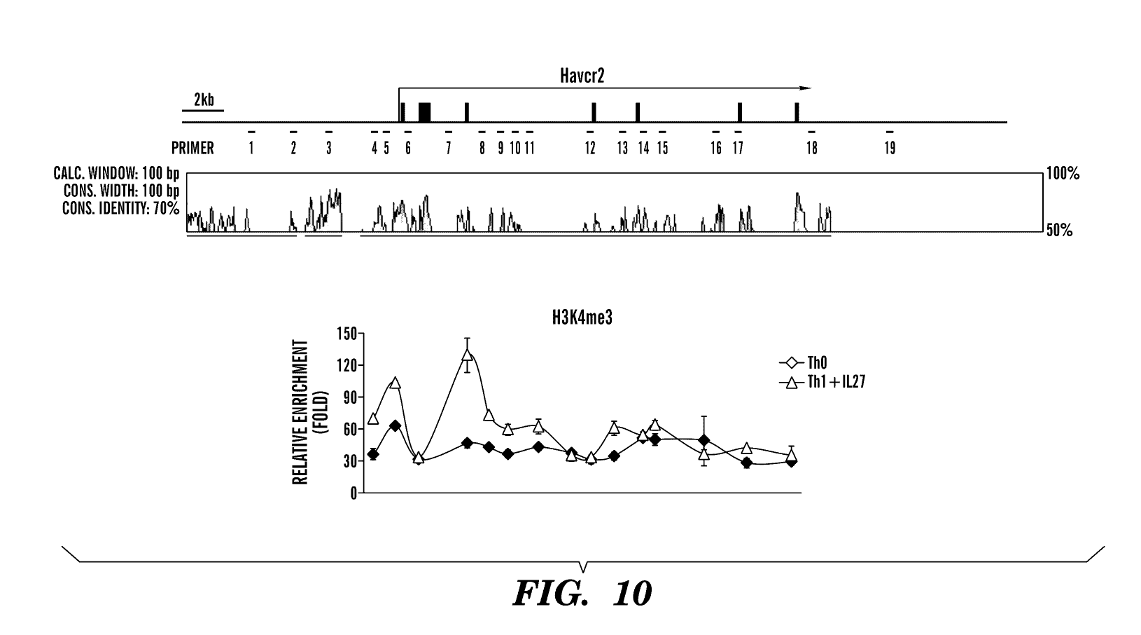

To understand the mechanism of IL-27-mediated activation of Tim-3 transcription, chromatin immunoprecipitation (ChIP) assays were performed to examine histone modification of the Tim-3 locus. The ChIP products were subjected to quantitative PCR using primer sets that cover the whole Tim-3 locus (FIG. 5A, Table 1). After comparing histone H3 acetylation (H3Ac) of the Tim-3 locus using chromatin prepared from in vitro differentiated Th0- and IL-27-treated Th1 cells, it was found that IL-27-treated Thl cells exhibited significantly higher enrichment of histone H3 acetylated at N-terminal lysine residues (H3Ac) in the promoter region and in introns 1, 3, and 5 of the Tim-3 locus (FIG. 5B). Similarly, IL-27-treated Thl cells also exhibited increased enrichment of histone H3 trimethylation at lysine 4 (H3K4me3), another permissive chromatin modeling marker, in the Tim-3 locus (FIG. 10).

TABLE 1
TIM-3 ChIP PCR Primers

| SEQ | | SEQ | |
|---|---|---|---|
| ID | | ID | |
| NO.: | Forward primers | NO.: | Reverse primers |
| 6 | ATTCAGTCCACCGTCTTTGC | 7 | GAACACAATGTAATTTTATCGTAATGG |
| 8 | CCTGAAGCTCACCAAACCTC | 9 | TGGCAGTCTTTGCTTCCTTT |
| 10 | GATCCCGCATTTTTAAGAGG | 11 | GCTGCACTGTTGCGACTTC |
| 12 | CTCTCAGGAAGGGCTGACAC | 13 | GGAAGGGGGACTTTGAACAT |
| 14 | AGTGCCTTGCAGGGTGTATC | 15 | TCCTGAGTCCCCAGAATCAC |
| 16 | AAGGAGGAGGGATGTCCTGT | 17 | ACCAGACCAGGAACGATGAC |
| 18 | TGTCAACTGGTTGCTTGCTC | 19 | AAGATGCCGCAGGATATTTG |
| 20 | AAGGCTCACAGCATCGTCTT | 21 | CTTCTGGGACAGCTTTCAGC |
| 22 | aacaaaaccaaaTCAAACCAAA | 23 | TCCTGGGGAACTCAAGACTG |
| 24 | GTTGCTGGGTGAAGCTCTTG | 25 | CCGCAACTGTTCTAAAGGGTA |
| 26 | AAAAGACTGCGAACCACCAT | 27 | GCTTGGGACCACCCTAATCT |
| 28 | CTAGGCACCTCAGCCTTTTG | 29 | GGAGGGTCACCAGTGTCTGT |
| 30 | GGGGGCAGGTGAGATAAAAT | 31 | CCCTAGTTCAAATCCCAGCA |
| 32 | TGTGGGCACATAAAATAAAGG | 33 | AACTGGCAGCATTTGGAAAG |
| 34 | AGATCCCAATGTTCCCATCA | 35 | TGAACACCAGAGATGGCCTA |
| 36 | CATGTGTTGCTTGCTTGCTT | 37 | GATCGGGTTGGTGTCAAAAC |
| 38 | AAGGGCTGTCCTGAAAGTCA | 39 | TCCTAAGCACGAGGCTTGTT |
| 40 | CATTCCTGGAGGAAACTGGA | 41 | ATAGATGGGAGCCAGCACAG |
| 42 | ACTGCCCAGGAGTCATCTGT | 43 | CCCAAAGATTTGATCCCTCA |

To To test whether NFIL3 is required for permissive chromatin modification in the Tim-3 locus in IL-27-treated Th1 cells, we compared H3Ac enrichment in the Tim-3 locus in IL-27-treated Th1 cells derived from WT and NFIL3−/− mice. The absence of NFIL3 resulted in profound attenuation of H3Ac enrichment in intron 1 (FIG. 5C). Interestingly, similar reduction of H3Ac enrichment in intron 1 was also found in the absence of T-bet (FIG. 5D), indicating that intron 1 is critical for the regulation of Tim-3 transcription.

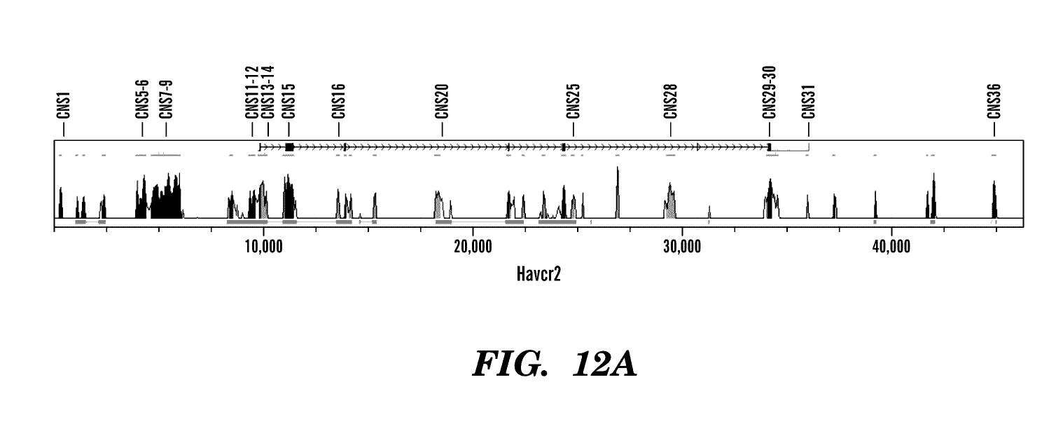

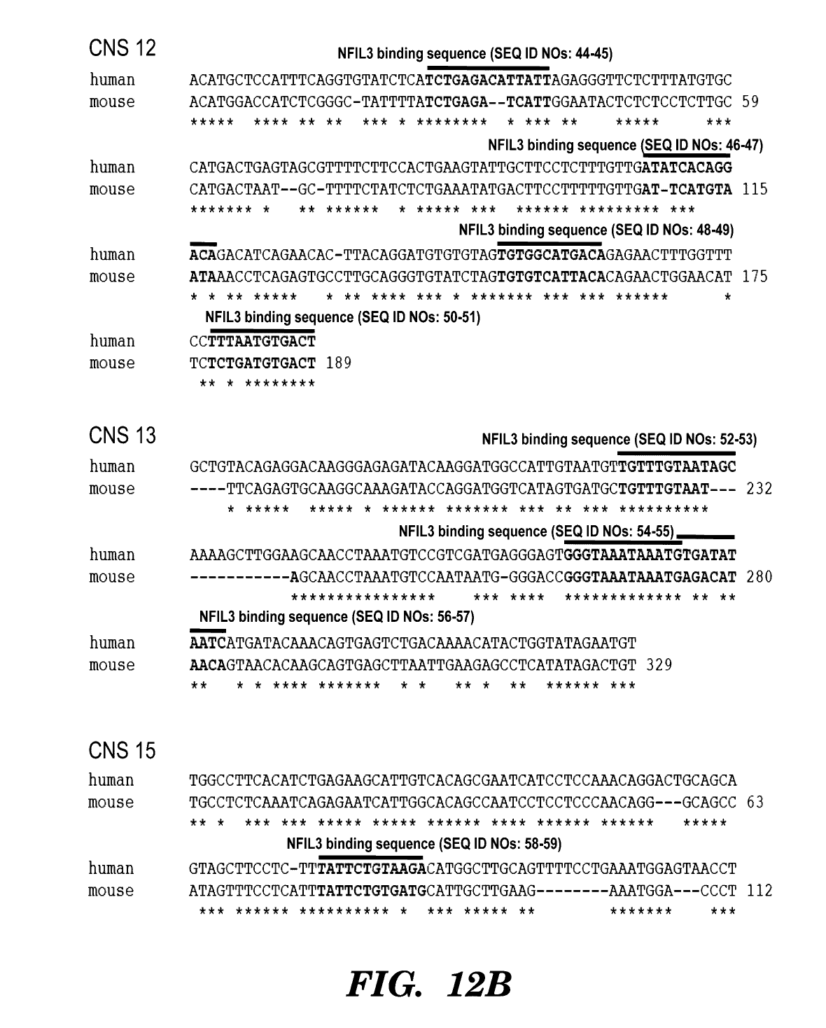

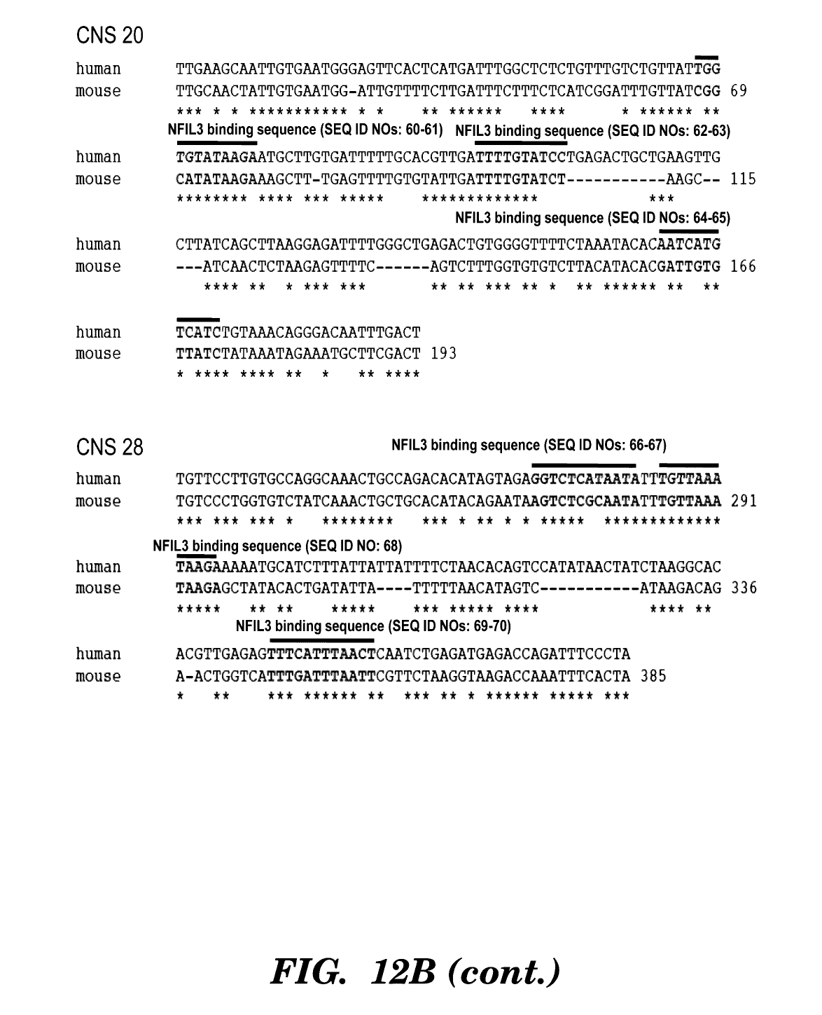

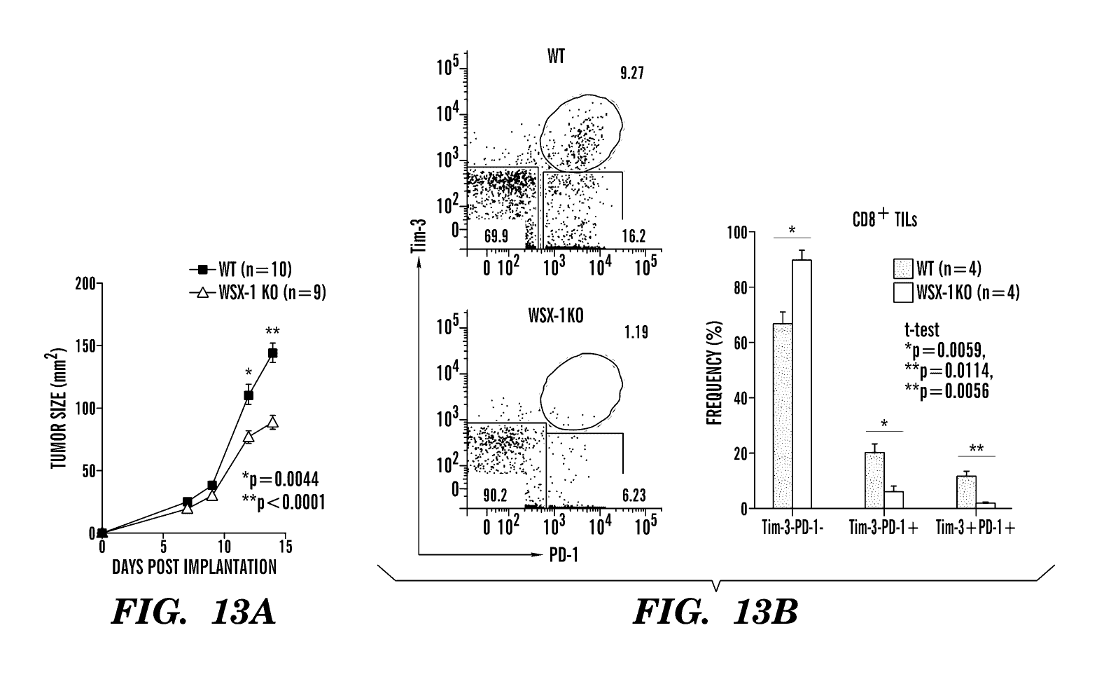

Our previous Chromatin co-immunoprecipitation (ChIP)-quantitative PCR (QPCR) demonstrated that T-bet binds to the Tim-3 proximal promoter region (T-bet, a Th1 transcription factor regulates the expression of Tim-3, Anderson AC, et al., Eur J Immunol. 2010 March; 40(3):859-66). To find out the NFIL3 binding region in the Tim-3 locus, we analyzed approximately 40 kb of genomic DNA sequence in the mouse Tim-3 locus aligned with the human homologous sequence. We identified multiple conserved non-coding sequence (CNS)s, defined as having 70% or greater identity over at least 100 bp stretches upstream and down-stream of the Tim-3 locus (FIG. 12A). To validate these putative binding regions in the Tim-3 locus, we conducted ChIP-QPCR to determine NFIL3 enrichment in the mouse Tim-3 locus using chromatin samples prepared from WT and NFIL3−/− CD4+ Th1 cells cultured in the presence of IL-27. We found about five potential NFIL-3 binding sites scattered in the Tim-3 proximal promoter region and introns 1, 3, 4, and 5 (FIG. 5E). Interestingly, these putative NFIL3 interaction sites overlap or are adjacent to regions that are involved in IL-27/IL-12-mediated permissive chromatin modification in the Tim-3 locus (FIGS. 5B, 5C). Many of the NFIL3 enrichment regions, including these in proximal promoter, introns 1, 3, and 5 overlapped with putative NFIL3 binding sites found in CNSs 12, 13, 15, 20, and 28 (FIGS. 5E, 12A, 12B; SEQ ID NOs: 36-62), indicating that NFIL3 directly regulatesTim-3 transcriptional regulation for both human and mouse Tim-3 expression. The fact that the majority of NFIL3 binding sites were found in the introns indicate that NFIL3 is predominantly related with the permissive chromatin remodeling for the optimal transcription of Tim-3. This is also supported by our observation of the reduced H3Ac enrichment in the Tim-3 locus in the absence of NFIL3 (FIG. 5B).

Since both NFIL3 and T-bet binding sites are found in the proximal promoter region, and NFIL3−/− and T-bet−/− T cells both showed reduced H3Ac enrichment in the adjacent intron 1, we then performed co-immunoprecipitation assays. The results indicate that NFIL3 and T-bet physically interact each other (FIG. 5F). Therefore, such interactions provide a physical basis for the functional synergy between these two transcription factors to drive Tim-3, and possibly IL-10, transcription. Taken together, the data described herein demonstrate that IL-27-enhanced Tim-3 expression in Thl cells is dependent on induction of NFIL3 and T-bet, which then interact with each other and bind to cis-regulatory regions in the Tim-3 locus thereby facilitating histone H3 acetylation for optimal expression of Tim-3.

### IL-27R Deficient Mice (WSX-1−/−) are Resistant to Tumor Growth

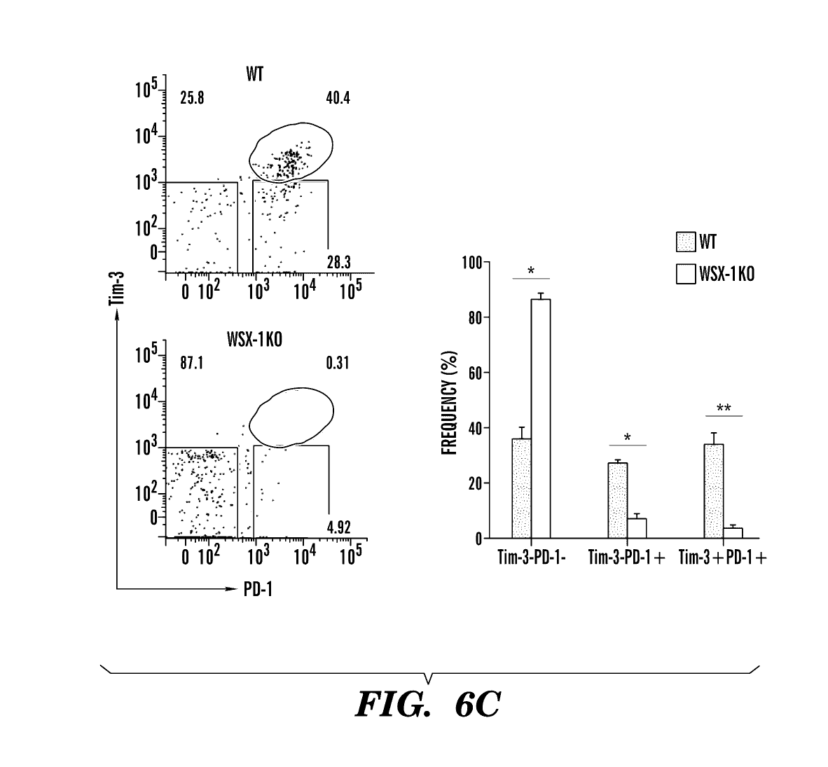

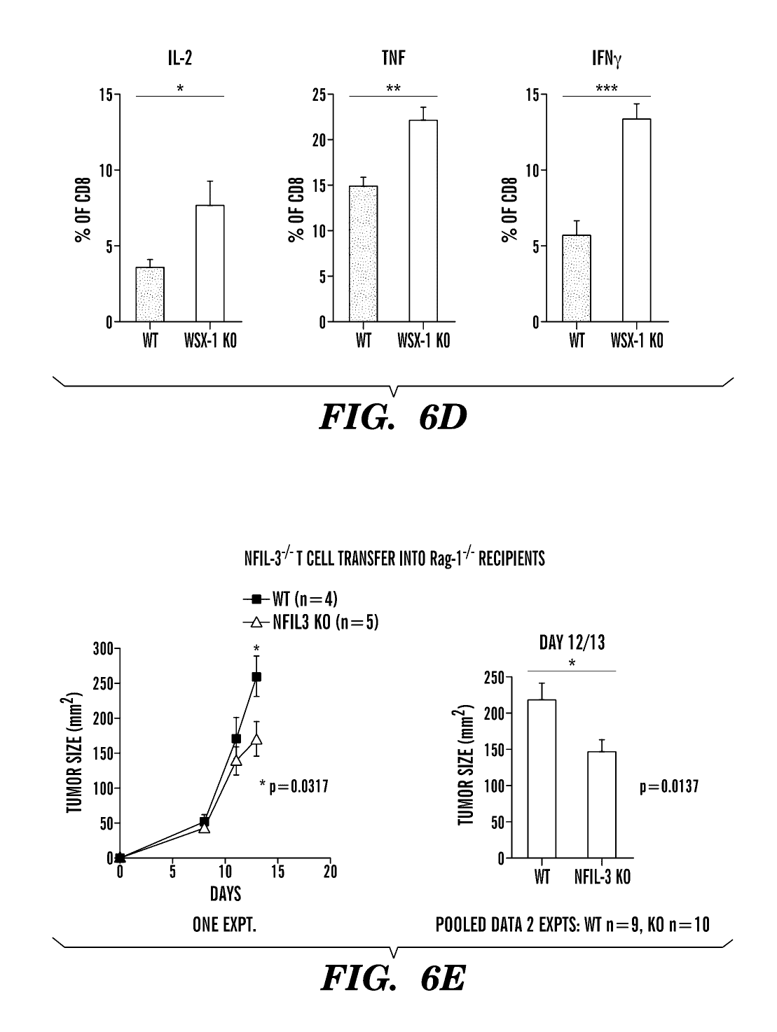

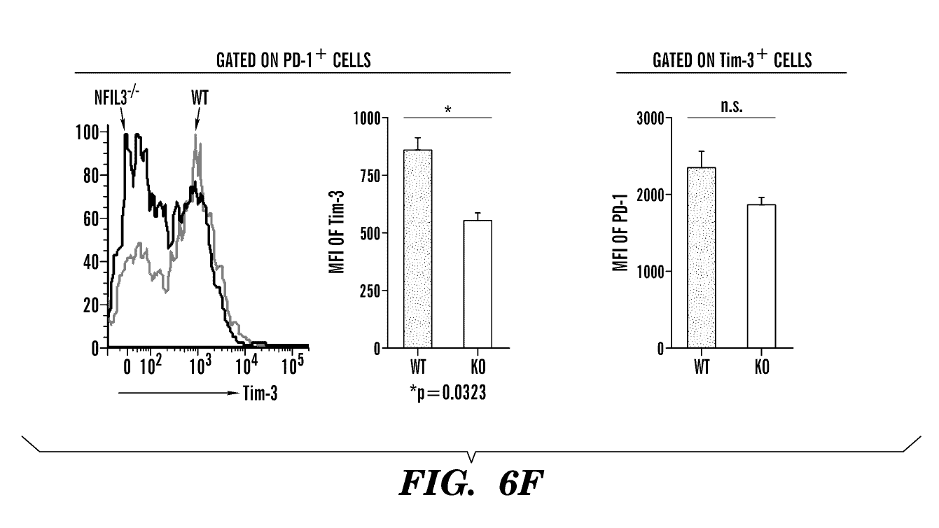

The in vitro studies described herein identify a mechanism of IL-27-mediated inhibition of effector Th1 and Tc1 cells by which IL-27-induced NFIL3 drives Tim-3 and IL-10, but suppresses IL-2 expression. Recently, Tim-3 has been reported to play a key role the development of exhausted T cells in chronic conditions such as cancer (14-16). Given that exhausted T cells fail to produce IL-2 but increased the expression of IL-10, the role of IL-27 in regulation of T cell responses in cancer was examined. To do this, Bl6F10 melanoma cells were implanted into C57BL/6 and WSX-1−/− mice and tumor growth in the recipient mice monitored. WSX-1−/− mice exhibited dramatically reduced tumor burden (FIG. 6A) and the tumor-infiltrating lymphoctyes (TILs) from these mice exhibit significantly reduced NFIL3 expression (FIG. 6B). Moreover, while CD8+ TILs from wild type tumor-bearing mice exhibited Tim-3+PD-1+CD8+ TILs that have been shown to exhibit exhausted phenotype, the same population could not be detected in WSX-1−/− recipients (FIG. 6C). Importantly, peripheral CD8+ T cells from WSX-1−/− mice exhibited much higher production of IL-2, IFN-γ and TNF, indicating that WSX-1−/− CD8+ T cells have more robust activation than wild type CD8+ T cells from control recipients (FIG. 6D). Thus, in the absence of IL-27 signaling, TILs in tumor-bearing animals failed to induce Tim-3 expression and to develop T cell exhaustion. Consequently, WSX1−/− mice better control tumor burden.

The role of NFIL3 in the regulation of exhaustion of tumor infiltrated T cells (TILs) was further studies. Since NFIL3−/− mice lack NK cells, adoptive transfer of total T cells into Rag-1−/− recipients was performed and subsequently implanted with Bl6F10 melanoma. Results showed that mice that received NFIL3−/− T cells had reduced tumor burden (FIG. 6E). In addition, TILs derived from NFIL3−/− T cell-transferred recipients exhibited lower percentage of exhausted Tim-3+PD-1+ population (FIG. 6F). These results demonstrate that NFIL-3 is required to the induction of T cell exhaustion.

### Ectopic Expression of NFIL-3 in CD4+ T Cells Reduces the Severity of Gut Inflammation.

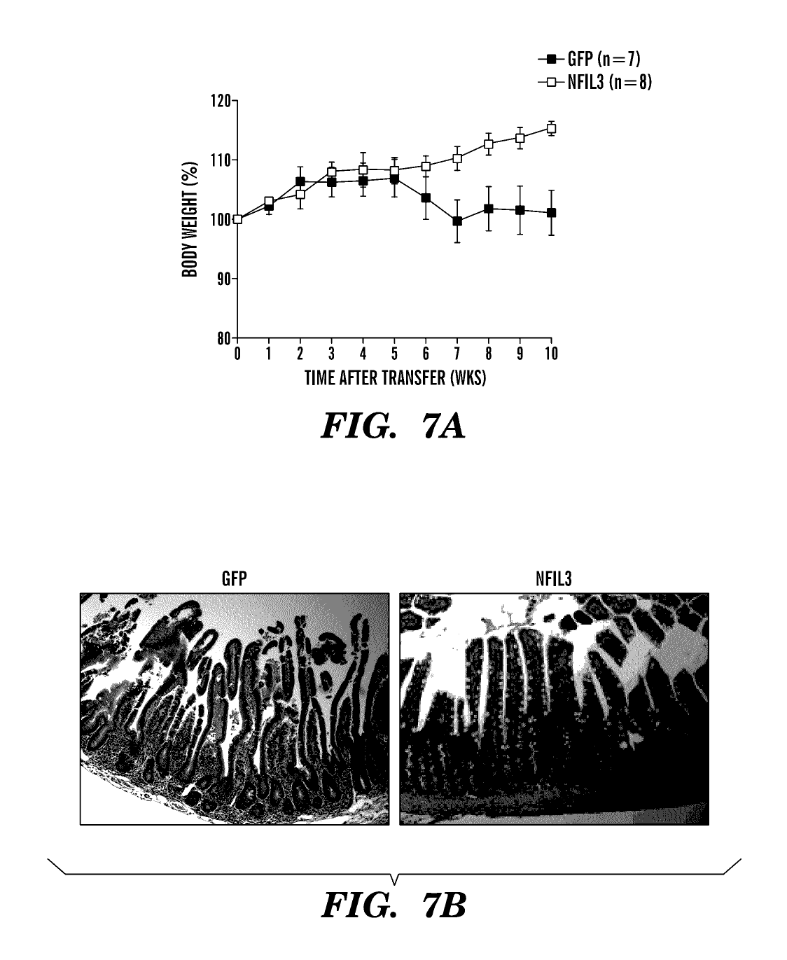

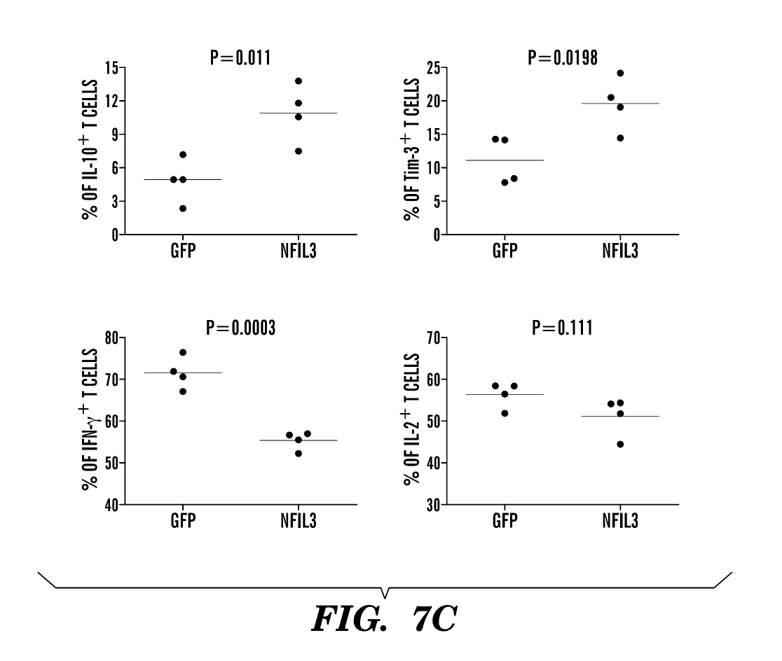

Our data described herein demonstrates that NFIL-3 plays a key role in the IL-27 signaling pathway to regulate the suppressive effect during the development of T cell exhaustion. Since ectopic expression of NFIL-3 in CD4+ T cells induced Tim-3 and IL-10 expression, we tested whether overexpression of NFIL-3 can dampen T cell immunity by inducing an exhaustion-like phenotype. We therefore transduced naïve CD4+ T cells with NFIL-3-expressing retrovirus (NFIL-3) and transferred these cells into Rag1−/− recipient mice to induce gut inflammation. Recipient mice that received NFIL-3-transduced CD4+ T cells, but not empty virus-transduced (GFP) CD4+ T cells, failed to develop wasting disease 10 weeks after transfer (FIG. 7A). Histological analysis further revealed that 5 out of 7 recepient mice that received empty virus-tranduced CD4+ T cells showed inflammation in small intestine, whereas only 2 out of 8 mice receiving NFIL-3-transduced CD4+ T cells showed similar inflammation in small intestine (FIG. 7B). We also found that NFIL-3-transduced T cells were localized in Peyer's patches, indicating that failure to induce inflammation in the small intestine was not due to NFIL-3 expression-induced cells death. In addition, when we harvested GFP- and NFIL-3-transduced cells from recipient mice 6 weeks post transfer, we found that NFIL-3-transduced cells from mesenteric lymph nodes exhibited significantly higher Tim-3 expression and IL-10 production, but IFN-γ production was reduced. There was a clear trend of reduction of IL-2 (FIG. 7C). Collectively, the date described herein demonstrate that effector function of NFIL-3-transduced CD4+ T cells was suppressed in vivo, thereby attenuating homeostatic proliferation-induced inflammation in gut.

It has been well established that interaction between Tim-3 and its ligand galectin-9 inhibits Th1 responses (11, 23, 24) and induces peripheral tolerance (25, 26). More importantly, Tim-3 plays a key role of in the regulation of T cell exhaustion during chronic viral infections. Elevated expression of Tim-3 helps to maintain exhausted phenotype in HIV-specific CD4+ and CD8+ T cells from individuals with progressive chronic HIV infection. Blockade of the interaction between Tim-3 and its ligand galectin-9 enhanced proliferation and cytokine production in HIV-1-specific CD8+ T cell (12). Similar role of Tim-3/galectin-9 signaling also involves the suppression of tumor infiltrating lymphocytes in cancers (14-16), further highlighting the biology of Tim-3 in controlling T cell immunity during chronic conditions.

A previous study indicated that IL-12 is required for Tim-3 expression (17). Such IL-12-induced Tim-3 expression was later found associated with T cell exhaustion in follicular B cell non-Hodgkin lymphoma (27). However, as the key transcription factor that is induced by IL-12, T-bet is unlikely the driving factor for the increased Tim-3 expression during chronic infection, since T-bet is critical for effector T cell differentiation and its expression is actually downregulated during chronic viral infection (22). T-bet, as a priming factor, can be necessary for permissive chromatin modification in the Tim-3 locus during the early stage of T cell activation. Optimal induction of Tim-3 expression still needs additional regulators. Indeed, the common y-chain (ye) cytokines such as IL-2, IL-7, and IL-21 were found to induce Tim-3 expression on human peripheral T cells via PI3K-Akt dependent pathway, providing another mechanism associated with Tim-3 expression during chronic viral infection (28).

IL-27 is demonstrated herein as the most potent Tim-3 inducer on naïve T cells. Mechanistically, IL-27 induces Tim-3 expression through the induction of T-bet, which overlaps the pathway with IL-12 signaling via STAT1-dependent way. Further, IL-27 also strongly induces the expression of NFIL-3, which involves the induction of Tim-3 via STAT3-dependent pathway. Interestingly, elevated NFIL-3 expression was found in terminal differentiated Th1 cells (21). NFIL-3 and T-bet synergistically induce Tim-3 expression by introducing permissive chromatin modification in the Tim-3 locus. As one of the most potent cytokines to induce NFIL-3 expression, IL-27-induced Tim-3 expression is likely due to its potency to induce both T-bet and NFIL-3. Importantly, the data described herein as well as others' also demonstrated that T-bet and NFIL-3 are essential for IL-27-mediated IL-10 expression (20, 21). Increased IL-10 expression was recently found as an important cytokine to suppress viral antigen-specific CD8+ T cells and induction of T cells exhaustion during chronic viral infection (29, 30). Given the fact that IL-27-induced IL-10 expression in Th1 cells is highly associated with Tim-3+ population, Tim-3 and IL-10 work together to provide a strong inhibitory signal to dampen T cell immunity. Indeed, as demonstrated herein, IL-27R deficient mice exhibited reduced tumor burden, which was accompanied by enhanced CTL function, increased proinflammatory cytokine production, and downregulated expression of Tim-3 and PD-1. Such in vivo effect is at least partially mediated by NFIL-3 at the downstream of IL-27 signaling.

NFIL-3 was identified as a master transcription factor for NK cell and CD8+ dendritic cell development (31-33). Recent studies began to reveal the regulatory function of NFIL-3 in T cell immunity. It has been known that NFIL-3 involves Th2 cytokine production (34) and IL-4-mediated IgE class switching (35). One interesting phenotype in NFIL-3 deficient mice is a profound defect in IL-10 production in various T cell subsets, indicating NFIL-3 can serve as an important anti-inflammatory regulator (21). Indeed, NFIL-3 deficiency resulted in more severe EAE and adoptively transferred colitis (21). By contrast, as demonstrated herein, it was found that forced expression of NFIL-3 in T cells prevented gut pathology in adoptive transfer colitis by inducing exhaustion like phenotype in transferred T cells. The anti-inflammatory effect of NFIL-3 is important for suppressing T cell function. Indeed, NFIL-3 is required for expression of Tim-3 and other inhibitory receptors including LAG3. Interestingly, the regulatory function of NFIL-3 recapitulates the down stream events of IL-27 signaling IFN-y producing T cells. Therefore, NFIL-3 is an important functional modulator of IL-27-mediated anti-inflammatory effect.

IL-27 has been known for its anti-inflammatory function to control T cell immunity in autoimmune diseases, bacterial infection, and CTL functions during acute viral infection (36). Various mechanisms have been found involving the suppressive effect of IL-27. Targeting master transcription regulators such as RORgt (37), GATA3 (38) by IL-27 signaling suppresses differentiation of effector Th17 and Th2 cells. IL-27 also induces PD-Ll expression on naïve CD4+ T cells suppress Th17 cells in trans through a PD-1-PD-L1 interaction (39). Importantly, IL-27-mediated IL-10 production is critical for suppression of a variety of effector T cell subsets (19, 37, 40). Herein, it is demonstrated that IL-27-induced Tim-3 expression can serve a key mechanism of suppressing IFNγ-producing T cells. Providing a critical role of Tim-3 in the induction of T cell exhaustion in cancers, the studies described herein provide a yet unappreciated role of IL-27 signaling in anti-tumor immunity. Thus, targeting the IL-27 pathway can used as a therapeutic approach in cancer treatment.

### Materials and Methods

Mice. STAT1−/− mice and 129S wild type mice were purchased from Taconic. Rag-1−/− mice, T-bet−/− mice, and C57BL/6 mice were purchased from The Jackson Laboratory. STAT3fl/fl×CD4-Cre conditional knockout (STAT3 cko) mice were provided by Dr. John O'Shea at NIH. NFIL-3−/− mice were provided by Dr. Tak Mak at University of Toronto. WSX-1−/− mice are commercially available from The Jackson Laboratory.

All mice were bred and kept in pathogenic free conditions. Animal experiments were done in accordance with the guidelines of the Institutional Animal Care and Use Committee (IACUC) at Harvard Medical School.

Cell isolation and culture. Total CD4+ T cells from different lines of mice were first enriched by positive selection using CD4⁺ T cell isolation reagent from Miltenyi Biotec. Naïve CD4⁺ (CD4⁺CD62L⁺) T cells were stained by PE-anti-CD4 and APC-anti-62L antibodies and were sorted by BD FACSARIA (BD Biosciences). The cells were then activated with plate-bound anti-CD3 (1 mg/ml; 145-2C11) and anti-CD28 (1 mg/ml; PV-1) (both were made in house) for 2 days. Th1 cells were cultured under the presence of IL-12 (10 ng/ml). In some conditions, 25 ng/ml of IL-27 was added.

Retroviral transduction. cDNAs encoding mouse NFIL-3 and T-bet were subcloned into modified pMSCV vector that bicistronically expresses GFP (for NFIL-3), and Thy1.1 (for Thy1.1). Retroviruses were packed in 293 T cells and were used to transduce mouse naïve CD4⁺ T cells activated by plate-bound anti-CD3 and anti-CD28 antibodies.

Intracellular cytokine staining. Naïve CD4+ T cells were activated by plate-bound anti-CD3 and anti-CD28 antibodies for 2 days. Cells were then rested for 3 days, and restimulated with 0.1 mg/ml of plate-bound anti-CD3 and anti-CD28 for 24 hours before they were subjected to PMA and ionomycin stimulation in the presence of GOLGI STOP™ (BD Biosciences) for intracellular cytokine detection. All data were collected on LSR II (BD BIOSCIENCES) or CALIBOr (BD BIOSCIENCES) and analyzed by FLOWJO software (TREE STAR, INC).

ChIP assays. Naive CD4⁺ T cells from C57BL/6 mice were purified by naïve CD4+ T cell negative selection kit (Miltenyi Biotec), and were activated by plate-bound anti-CD3 and anti-CD8 (2 mg/ml each) under Th0 or Th1+IL-27 condition for 2 days. Cells were rested for additional 3 days and were restimulated with 0.1 mg/ml of plate-bound anti-CD3 and anti-CD28 for 24 hours before they were subjected to chromatin preparation for the ChIP analysis. Chromatin fractions and chromatin IP were performed using SIMPLECHIP™ Enzymatic Chromatin IP Kit (CELL SIGNALING TECHNOLOGY). Antibody against NFIL-3 (C-18) were purchased from SANTA CRUZ BIOTECHNOLOGY; anti-acetylated Histone 3 antibody was purchased from MILLIPORe (06-599); and anti-Histone H3 trimethyl-lysine 4 antibody was purchased from ABCAM (ab8580).

Real-time PCR analysis. RNA was extracted with RNEASY PLUS kits (QIAGEN) and cDNA was made by ISCRIPT (BIORAD). All of the Real-time PCR probes were purchased from APPLIED BIOSYSTEMS. Quantitative PCR were performed by the GENEAMP7500 Sequence Detection System and VIIA™ 7 Real-Time PCR System (APPLIED BIOSYSTEMS).

Tumor challenge and phenotypic/functional studies. B16F10 melanoma (CRL-6475) cell line was purchased from ATCC. 1×10⁵ or 5×10⁵ cells were injected subcutaneously at the flanks of the mice. Tumors were measured in two dimensions by caliper as the product of two perpendicular diameters. TILS were isolated as previously described on day 14 to day 20 post tumor implantation as they were reaching 200 mm² in size (14). Tumors dissected from the mice were dissociated either manually or by GENTLEMACS dissociator (MILTENYI BIOTEC, CA) and then treated with collagenase D before PERCOLL gradient separation. Lymphocytes from ipsilateral inguinal lymph nodes (draining lymph nodes; DLN) were also separated in some experiments. Single cells suspensions were stained for CD8, CD4, Tim-3, and PD-1. For functional assay, intracellular cytokine staining was conducted as described before (14). For gene expression analysis, CD8⁺7AAD⁻ TILs of B16 melanoma on WT C57BL/c mice or WSX-1^{-/-} mice were sorted by BD FACSAria after magnetic separation by DYNABEADS FLOWCOMP Mouse CD8 (INVITROGEN). CD44^{low} CD62L^{high} memory CD8⁺ splenocytes from B6 non-tumor bearing mice were also sorted as a control. RNA from sorted CD8⁺ cells were then extracted and reverse transcribed to cDNA. Gene expressions were quantified by TAQMAN PCR.

Colitis model. Naïve CD4⁺ T cells from C57BL/6 mice were subjected to TcR activation by anti-CD3 and anti-CD28 antibodies. Cells were subsequently transduced with NFIL-3-expression retrovirus or GFP empty retrovirus in the next day. On day 5 after activation, GFP positive T cells were sorted by BD FACSARIA (BD BIOSCIENCES) and were transferred intraperitoneally into Rag-1^{-/-} recipient mice. Body weight and symptoms of disease were monitored up to 10 weeks. At the end point of the experiment, mice were sacrificed. Intestines were fixed with 10% Formalin and sections were stained with hematoxylin and eosin. To analyze Tim-3 expression and cytokine production, NFIL-3 or GFP transduced cells were i.p. injected to C57BL/6 mice. Mice were sacrificed 6 weeks after injection. Cells from spleen and mesenteric lymph nodes were restimulated with PMA/ionomycin in the presence of GOLGI STOP™ (BD BIOSCIENCES) for detection of cytokine production and Tim-3 expression by flow cytometry.

### REFERENCES

1. Wherry, E. J. T cell exhaustion. Nat Immunol 12:492-499.
2. Shin, H., and E. J. Wherry. 2007. CD8 T cell dysfunction during chronic viral infection. Curr Opin Immunol 19:408-415.
3. Virgin, H. W., E. J. Wherry, and R. Ahmed. 2009. Redefining chronic viral infection. Cell 138:30-50.
4. Barber, D. L., E. J. Wherry, D. Masopust, B. Zhu, J. P. Allison, A. H. Sharpe, G. J. Freeman, and R. Ahmed. 2006. Restoring function in exhausted CD8 T cells during chronic viral infection. Nature 439:682-687.
5. Petrovas, C., J. P. Casazza, J. M. Brenchley, D. A. Price, E. Gostick, W. C. Adams, M. L. Precopio, T. Schacker, M. Roederer, D. C. Douek, and R. A. Koup. 2006. PD-1 is a regulator of virus-specific CD8+ T cell survival in HIV infection. J Exp Med 203:2281-2292.
6. Trautmann, L., L. Janbazian, N. Chomont, E. A. Said, S. Gimmig, B. Bessette, M. R. Boulassel, E. Delwart, H. Sepulveda, R. S. Balderas, J. P. Routy, E. K. Haddad, and R. P. Sekaly. 2006. Upregulation of PD-1 expression on HIV-specific CD8+ T cells leads to reversible immune dysfunction. Nat Med 12:1198-1202.
7. Day, C. L., D. E. Kaufmann, P. Kiepiela, J. A. Brown, E. S. Moodley, S. Reddy, E. W. Mackey, J. D. Miller, A. J. Leslie, C. DePierres, Z. Mncube, J. Duraiswamy, B. Zhu, Q. Eichbaum, M. Altfeld, E. J. Wherry, H. M. Coovadia, P. J. Goulder, P. Klenerman, R. Ahmed, G. J. Freeman, and B. D. Walker. 2006. PD-1 expression on HIV-specific T cells is associated with T-cell exhaustion and disease progression. Nature 443:350-354.
8. Blackburn, S. D., H. Shin, W. N. Haining, T. Zou, C. J. Workman, A. Polley, M. R. Betts, G. J. Freeman, D. A. Vignali, and E. J. Wherry. 2009. Coregulation of CD8+ T cell exhaustion by multiple inhibitory receptors during chronic viral infection. Nat Immunol 10:29-37.
9. Jin, H. T., A. C. Anderson, W. G. Tan, E. E. West, S. J. Ha, K. Araki, G. J. Freeman, V. K. Kuchroo, and R. Ahmed. Cooperation of Tim-3 and PD-1 in CD8 T-cell exhaustion during chronic viral infection. Proc Natl Acad Sci USA 107:14733-14738.
10. Monney, L., C. A. Sabatos, J. L. Gaglia, A. Ryu, H. Waldner, T. Chernova, S. Manning, E. A. Greenfield, A. J. Coyle, R. A. Sobel, G. J. Freeman, and V. K. Kuchroo. 2002. Th1-specific cell surface protein Tim-3 regulates macrophage activation and severity of an autoimmune disease. Nature 415:536-541.
11. Zhu, C., A. C. Anderson, A. Schubart, H. Xiong, J. Imitola, S. J. Khoury, X. X. Zheng, T. B. Strom, and V. K. Kuchroo. 2005. The Tim-3 ligand galectin-9 negatively regulates T helper type 1 immunity. Nat Immunol 6:1245-1252.
12. Jones, R. B., L. C. Ndhlovu, J. D. Barbour, P. M. Sheth, A. R. Jha, B. R. Long, J. C. Wong, M. Satkunarajah, M. Schweneker, J. M. Chapman, G. Gyenes, B. Vali, M. D. Hyrcza, F. Y. Yue, C. Kovacs, A. Sassi, M. Loutfy, R. Halpenny, D. Persad, G. Spotts, F. M. Hecht, T. W. Chun, J. M. McCune, R. Kaul, J. M. Rini, D. F. Nixon, and M. A. Ostrowski. 2008. Tim-3 expression defines a novel population of dysfunctional T cells with highly elevated frequencies in progressive HIV-1 infection. J Exp Med 205:2763-2779.
13. McMahan, R. H., L. Golden-Mason, M. I. Nishimura, B. J. McMahon, M. Kemper, T. M. Allen, D. R. Gretch, and H. R. Rosen. Tim-3 expression on PD-1+ HCV-specific human CTLs is associated with viral persistence, and its blockade restores hepatocyte-directed in vitro cytotoxicity. J Clin Invest 120:4546-4557.
14. Sakuishi, K., L. Apetoh, J. M. Sullivan, B. R. Blazar, V. K. Kuchroo, and A. C. Anderson. Targeting Tim-3 and PD-1 pathways to reverse T cell exhaustion and restore anti-tumor immunity. J Exp Med 207:2187-2194.
15. Zhou, Q., M. E. Munger, R. G. Veenstra, B. J. Weigel, M. Hirashima, D. H. Munn, W. J. Murphy, M. Azuma, A. C. Anderson, V. K. Kuchroo, and B. R. Blazar. Coexpression of Tim-3 and PD-1 identifies a CD8+ T-cell exhaustion phenotype in mice with disseminated acute myelogenous leukemia. Blood 117:4501-4510.
16. Fourcade, J., Z. Sun, M. Benallaoua, P. Guillaume, I. F. Luescher, C. Sander, J. M. Kirkwood, V. Kuchroo, and H. M. Zarour. Upregulation of Tim-3 and PD-1 expression is associated with tumor antigen-specific CD8+ T cell dysfunction in melanoma patients. J Exp Med 207:2175-2186.
17. Anderson, A. C., G. M. Lord, V. Dardalhon, D. H. Lee, C. A. Sabatos-Peyton, L. H. Glimcher, and V. K. Kuchroo. T-bet, a Th1 transcription factor regulates the expression of Tim-3. Eur J Immunol 40:859-866.
18. Awasthi, A., Y. Carrier, J. P. Peron, E. Bettelli, M. Kamanaka, R. A. Flavell, V. K. Kuchroo, M. Oukka, and H. L. Weiner. 2007. A dominant function for interleukin 27 in generating interleukin 10-producing anti-inflammatory T cells. Nat Immunol 8:1380-1389.
19. Batten, M., N. M. Kljavin, J. Li, M. J. Walter, F. J. de Sauvage, and N. Ghilardi. 2008. Cutting edge: IL-27 is a potent inducer of IL-10 but not FoxP3 in murine T cells. J Immunol 180:2752-2756.
20. Fitzgerald, D. C., G. X. Zhang, M. El-Behi, Z. Fonseca-Kelly, H. Li, S. Yu, C. J. Saris, B. Gran, B. Ciric, and A. Rostami. 2007. Suppression of autoimmune inflammation of the central nervous system by interleukin 10 secreted by interleukin 27-stimulated T cells. Nat Immunol 8:1372-1379.
21. Motomura, Y., H. Kitamura, A. Hijikata, Y. Matsunaga, K. Matsumoto, H. Inoue, K. Atarashi, S. Hori, H. Watarai, J. Zhu, M. Taniguchi, and M. Kubo. The transcription factor E4BP4 regulates the production of IL-10 and IL-13 in CD4+ T cells. Nat Immunol 12:450-459.
22. Kao, C., K. J. Oestreich, M. A. Paley, A. Crawford, J. M. Angelosanto, M. A. Ali, A. M. Intlekofer, J. M. Boss, S. L. Reiner, A. S. Weinmann, and E. J. Wherry. Transcription factor T-bet represses expression of the inhibitory receptor PD-1 and sustains virus-specific CD8+ T cell responses during chronic infection. Nat Immunol 12:663-671.
23. Klibi, J., T. Niki, A. Riedel, C. Pioche-Durieu, S. Souquere, E. Rubinstein, S. Le Moulec, J. Guigay, M. Hirashima, F. Guemira, D. Adhikary, J. Mautner, and P. Busson. 2009. Blood diffusion and Th1-suppressive effects of galectin-9-containing exosomes released by Epstein-Barr virus-infected nasopharyngeal carcinoma cells. Blood 113:1957-1966.
24. Sehrawat, S., A. Suryawanshi, M. Hirashima, and B. T. Rouse. 2009. Role of Tim-3/galectin-9 inhibitory interaction in viral-induced immunopathology: shifting the balance toward regulators. J Immunol 182:3191-3201.
25. Sanchez-Fueyo, A., J. Tian, D. Picarella, C. Domenig, X. X. Zheng, C. A. Sabatos, N. Manlongat, O. Bender, T. Kamradt, V. K. Kuchroo, J. C. Gutierrez-Ramos, A. J. Coyle, and T. B. Strom. 2003. Tim-3 inhibits T helper type 1-mediated auto- and alloimmune responses and promotes immunological tolerance. Nat Immunol 4:1093-1101.
26. Sabatos, C. A., S. Chakravarti, E. Cha, A. Schubart, A. Sanchez-Fueyo, X. X. Zheng, A. J. Coyle, T. B. Strom, G. J. Freeman, and V. K. Kuchroo. 2003. Interaction of Tim-3 and Tim-3 ligand regulates T helper type 1 responses and induction of peripheral tolerance. Nat Immunol 4:1102-1110.
27. Yang, Z. Z., D. M. Grote, S. C. Ziesmer, T. Niki, M. Hirashima, A. J. Novak, T. E. Witzig, and S. M. Ansell. IL-12 upregulates TIM-3 expression and induces T cell exhaustion in patients with follicular B cell non-Hodgkin lymphoma. J Clin Invest 122:1271-1282.
28. Mujib, S., R. B. Jones, C. Lo, N. Aidarus, K. Clayton, A. Sakhdari, E. Benko, C. Kovacs, and M. A. Ostrowski. Antigen-independent induction of Tim-3 expression on human T cells by the common gamma-chain cytokines IL-2, IL-7, IL-15, and IL-21 is associated with proliferation and is dependent on the phosphoinositide 3-kinase pathway. J Immunol 188:3745-3756.
29. Ejrnaes, M., C. M. Filippi, M. M. Martinic, E. M. Ling, L. M. Togher, S. Crotty, and M. G. von Herrath. 2006. Resolution of a chronic viral infection after interleukin-10 receptor blockade. J Exp Med 203:2461-2472.
30. Brooks, D. G., M. J. Trifilo, K. H. Edelmann, L. Teyton, D. B. McGavern, and M. B. Oldstone. 2006. Interleukin-10 determines viral clearance or persistence in vivo. Nat Med 12:1301-1309.
31. Gascoyne, D. M., E. Long, H. Veiga-Fernandes, J. de Boer, O. Williams, B. Seddon, M. Coles, D. Kioussis, and H. J. Brady. 2009. The basic leucine zipper transcription factor E4BP4 is essential for natural killer cell development. Nat Immunol 10:1118-1124.
32. Kamizono, S., G. S. Duncan, M. G. Seidel, A. Morimoto, K. Hamada, G. Grosveld, K. Akashi, E. F. Lind, J. P. Haight, P. S. Ohashi, A. T. Look, and T. W. Mak. 2009. Nfi13/E4bp4 is required for the development and maturation of NK cells in vivo. J Exp Med 206:2977-2986.
33. Kashiwada, M., N. L. Pham, L. L. Pewe, J. T. Harty, and P. B. Rothman. NFIL3/E4BP4 is a key transcription factor for CD8alpha dendritic cell development. Blood 117:6193-6197.
34. Kashiwada, M., S. L. Cassel, J. D. Colgan, and P. B. Rothman. NFIL3/E4BP4 controls type 2 T helper cell cytokine expression. EMBO J 30:2071-2082.
35. Kashiwada, M., D. M. Levy, L. McKeag, K. Murray, A. J. Schroder, S. M. Canfield, G. Traver, and P. B. Rothman. IL-4-induced transcription factor NFIL3/E4BP4 controls IgE class switching. Proc Natl Acad Sci USA 107:821-826.
36. Sun, J., H. Dodd, E. K. Moser, R. Sharma, and T. J. Braciale. CD4+ T cell help and innate-derived IL-27 induce Blimp-1-dependent IL-10 production by antiviral CTLs. Nat Immunol 12:327-334.
37. Diveu, C., M. J. McGeachy, K. Boniface, J. S. Stumhofer, M. Sathe, B. Joyce-Shaikh, Y. Chen, C. M. Tato, T. K. McClanahan, R. de Waal Malefyt, C. A. Hunter, D. J. Cua, and R. A. Kastelein. 2009. IL-27 blocks RORc expression to inhibit lineage commitment of Th17 cells. J Immunol 182:5748-5756.
38. Lucas, S., N. Ghilardi, J. Li, and F. J. de Sauvage. 2003. IL-27 regulates IL-12 responsiveness of naive CD4+ T cells through Stat1-dependent and -independent mechanisms. Proc Natl Acad Sci USA 100:15047-15052.
39. Hirahara, K., K. Ghoreschi, X. P. Yang, H. Takahashi, A. Laurence, G. Vahedi, G. Sciume, A. O. Hall, C. D. Dupont, L. M. Francisco, Q. Chen, M. Tanaka, Y. Kanno, H. W. Sun, A. H. Sharpe, C. A. Hunter, and J. J. O'Shea. Interleukin-27 Priming of T Cells Controls IL-17 Production In trans via Induction of the Ligand PD-L1. Immunity 36:1017-1030.
40. Stumhofer, J. S., J. S. Silver, A. Laurence, P. M. Porrett, T. H. Harris, L. A. Turka, M. Ernst, C. J. Saris, J. J. O'Shea, and C. A. Hunter. 2007. Interleukins 27 and 6 induce STAT3-mediated T cell production of interleukin 10. Nat Immunol 8:1363-1371.

We claim:

1. A method for decreasing T-cell exhaustion in a subject in need thereof, comprising administering to a subject an effective amount of a pharmaceutical composition comprising an IL-27 inhibitor.

2. The method of claim 1, wherein the IL-27 inhibitor decreases IL-27 mediated NFIL-3 (nuclear factor, interleukin-3 regulated) induction or activation.

3. The method of claim 1, wherein the IL-27 inhibitor decreases NFIL-3 binding to a sequence at the TIM-3 locus selected from any one of SEQ ID NO: 46-SEQ ID NO: 70.

4. The method of claim 1, wherein the IL-27 inhibitor decreases histone acetylation at a sequence at the TIM-3 locus selected from any one of SEQ ID NO: 46-SEQ ID NO: 70.

5. The method of claim 1, wherein the IL-27 inhibitor is an anti-IL-27 antibody or antigen-binding fragment thereof, a small molecule IL-27 inhibitor, an RNA or DNA aptamer that binds or physically interacts with IL-27 or IL-27R, an IL-27 or IL-27 receptor structural analog, a soluble IL-27 receptor, an IL-27 specific antisense molecule, or an IL-27 specific siRNA molecule.

6. The method of claim 1, wherein the subject being administered the IL-27 inhibitor is diagnosed as having a cancer or tumor.

7. The method of claim 1, wherein the subject being administered the IL-27 inhibitor has a chronic immune condition that comprises a population of functionally exhausted T cells.


SEQUENCE LISTING (81 sequences)

SEQ ID NO: 1 - protein, 243 aa, Homo sapiens
MGQTAGDLGWRLSLLLLPLLLVQAGVWGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSALQGPAQVSWPQLLSTYRLLHSLELVLSRAVRELLLLSKAGHSVWPLGFPTLSPQP

SEQ ID NO: 2 - protein, 229 aa, Homo sapiens
MTPQLLLALVLWASCPPCSGRKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 3 - protein, 302 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTLQRDFTAAFPRMLTTRGHGPPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 4 - DNA, 10 bp, Unknown
  Description of Unknown Cellular or viral promoter sequence
rttaygtaay

SEQ ID NO: 5 - protein, 462 aa, Homo sapiens
MQLRKMQTVKKEQASLDASSNVDKMMVLNSALTEVSEDSTTGEELLLSEGSVGKNKSSACRRKREFIPDEKKDAMYWEKRRKNNEAAKRSREKRRLNDLVLENKLIALGEENATLKAELLSLKLKFGLISSTAYAQEIQKLSNSTAVYFQDYQTSKSNVSSFVDEHEPSMVSSSCISVIKHSPQSSLSDVSEVSSVEHTQESSVQGSCRSPENKFQIIKQEPMELESYTREPRDDRGSYTASIYQNYMGNSFSGYSHSPPLLQVNRSSSNSPRTSETDDGVVGKSSDGEDEQQVPKGPIHSPVELKHVHATVVKVPEVNSSALPHKLRIKAKAMQIKVEAFDNEFEATQKLSSPIDMTSKRHFELEKHSAPSMVHSSLTPFSVQVTNIQDWSLKSEHWHQKELSGKTQNSFKTGVVEMKDSGYKVSDPENLYLKQGIANLSAEVVSLKRLIATQPISASDSG

SEQ ID NO: 6 - DNA, 20 bp, Artificial Sequence
  Description of Artificial Sequence Synthetic primer
attcagtccaccgtctttgc

SEQ ID NO: 7 - DNA, 27 bp, Artificial Sequence
  Description of Artificial Sequence Synthetic primer
gaacacaatgtaattttatcgtaatgg

SEQ ID NO: 8 - DNA, 20 bp, Artificial Sequence
  Description of Artificial Sequence Synthetic primer
cctgaagctcaccaaacctc

SEQ ID NO: 9 - DNA, 20 bp, Artificial Sequence
  Description of Artificial Sequence Synthetic primer
tggcagtctttgcttccttt

SEQ ID NO: 10 - DNA, 20 bp, Artificial Sequence
  Description of Artificial Sequence Synthetic primer
gatcccgcatttttaagagg

SEQ ID NO: 11 - DNA, 19 bp, Artificial Sequence
  Description of Artificial Sequence Synthetic primer
gctgcactgttgcgacttc

SEQ ID NO: 12 - DNA, 20 bp, Artificial Sequence
  Description of Artificial Sequence Synthetic primer
ctctcaggaagggctgacac

SEQ ID NO: 13 - DNA, 20 bp, Artificial Sequence
  Description of Artificial Sequence Synthetic primer
ggaagggggactttgaacat

SEQ ID NO: 14 - DNA, 20 bp, Artificial Sequence
  Description of Artificial Sequence Synthetic primer
agtgccttgcagggtgtatc

SEQ ID NO: 15 - DNA, 20 bp, Artificial Sequence
  Description of Artificial Sequence Synthetic primer
tcctgagtccccagaatcac

SEQ ID NO: 16 - DNA, 20 bp, Artificial Sequence
  Description of Artificial Sequence Synthetic primer
aaggaggagggatgtcctgt

SEQ ID NO: 17 - DNA, 20 bp, Artificial Sequence
  Description of Artificial Sequence Synthetic primer
accagaccaggaacgatgac

SEQ ID NO: 18 - DNA, 20 bp, Artificial Sequence
  Description of Artificial Sequence Synthetic primer
tgtcaactggttgcttgctc

SEQ ID NO: 19 - DNA, 20 bp, Artificial Sequence
  Description of Artificial Sequence Synthetic primer
aagatgccgcaggatatttg

SEQ ID NO: 20 - DNA, 20 bp, Artificial Sequence
  Description of Artificial Sequence Synthetic primer
aaggctcacagcatcgtctt

SEQ ID NO: 21 - DNA, 20 bp, Artificial Sequence
  Description of Artificial Sequence Synthetic primer
cttctgggacagctttcagc

SEQ ID NO: 22 - DNA, 22 bp, Artificial Sequence
  Description of Artificial Sequence Synthetic primer
aacaaaaccaaatcaaaccaaa

SEQ ID NO: 23 - DNA, 20 bp, Artificial Sequence
  Description of Artificial Sequence Synthetic primer
tcctggggaactcaagactg

SEQ ID NO: 24 - DNA, 20 bp, Artificial Sequence
  Description of Artificial Sequence Synthetic primer
gttgctgggtgaagctcttg

SEQ ID NO: 25 - DNA, 21 bp, Artificial Sequence
  Description of Artificial Sequence Synthetic primer
ccgcaactgttctaaagggta

SEQ ID NO: 26 - DNA, 20 bp, Artificial Sequence
  Description of Artificial Sequence Synthetic primer
aaaagactgcgaaccaccat

SEQ ID NO: 27 - DNA, 20 bp, Artificial Sequence
  Description of Artificial Sequence Synthetic primer
gcttgggaccaccctaatct

SEQ ID NO: 28 - DNA, 20 bp, Artificial Sequence
  Description of Artificial Sequence Synthetic primer
ctaggcacctcagccttttg

SEQ ID NO: 29 - DNA, 20 bp, Artificial Sequence
  Description of Artificial Sequence Synthetic primer
ggagggtcaccagtgtctgt

SEQ ID NO: 30 - DNA, 20 bp, Artificial Sequence
  Description of Artificial Sequence Synthetic primer
gggggcaggtgagataaaat

SEQ ID NO: 31 - DNA, 20 bp, Artificial Sequence
  Description of Artificial Sequence Synthetic primer
ccctagttcaaatcccagca

SEQ ID NO: 32 - DNA, 21 bp, Artificial Sequence
  Description of Artificial Sequence Synthetic primer
tgtgggcacataaaataaagg

SEQ ID NO: 33 - DNA, 20 bp, Artificial Sequence
  Description of Artificial Sequence Synthetic primer
aactggcagcatttggaaag

SEQ ID NO: 34 - DNA, 20 bp, Artificial Sequence
  Description of Artificial Sequence Synthetic primer
agatcccaatgttcccatca

SEQ ID NO: 35 - DNA, 20 bp, Artificial Sequence
  Description of Artificial Sequence Synthetic primer
tgaacaccagagatggccta

SEQ ID NO: 36 - DNA, 20 bp, Artificial Sequence
  Description of Artificial Sequence Synthetic primer
catgtgttgcttgcttgctt

SEQ ID NO: 37 - DNA, 20 bp, Artificial Sequence
  Description of Artificial Sequence Synthetic primer
gatcgggttggtgtcaaaac

SEQ ID NO: 38 - DNA, 20 bp, Artificial Sequence
  Description of Artificial Sequence Synthetic primer
aagggctgtcctgaaagtca

SEQ ID NO: 39 - DNA, 20 bp, Artificial Sequence
  Description of Artificial Sequence Synthetic primer
tcctaagcacgaggcttgtt

SEQ ID NO: 40 - DNA, 20 bp, Artificial Sequence
  Description of Artificial Sequence Synthetic primer
cattcctggaggaaactgga

SEQ ID NO: 41 - DNA, 20 bp, Artificial Sequence
  Description of Artificial Sequence Synthetic primer
atagatgggagccagcacag

SEQ ID NO: 42 - DNA, 20 bp, Artificial Sequence
  Description of Artificial Sequence Synthetic primer
actgcccaggagtcatctgt

SEQ ID NO: 43 - DNA, 20 bp, Artificial Sequence
  Description of Artificial Sequence Synthetic primer
cccaaagatttgatccctca

SEQ ID NO: 44 - DNA, 14 bp, Homo sapiens
tctgagacattatt

SEQ ID NO: 45 - DNA, 12 bp, Mus sp.
tctgagatcatt

SEQ ID NO: 46 - DNA, 13 bp, Homo sapiens
atatcacaggaca

SEQ ID NO: 47 - DNA, 12 bp, Mus sp.
attcatgtaata

SEQ ID NO: 48 - DNA, 12 bp, Homo sapiens
tgtggcatgaca

SEQ ID NO: 49 - DNA, 12 bp, Mus sp.
tgtgtcattaca

SEQ ID NO: 50 - DNA, 12 bp, Homo sapiens
tttaatgtgact

SEQ ID NO: 51 - DNA, 12 bp, Mus sp.
tctgatgtgact

SEQ ID NO: 52 - DNA, 13 bp, Homo sapiens
tgtttgtaatagc

SEQ ID NO: 53 - DNA, 10 bp, Mus sp.
tgtttgtaat

SEQ ID NO: 54 - DNA, 13 bp, Homo sapiens
gggtaaataaatg

SEQ ID NO: 55 - DNA, 13 bp, Mus sp.
gggtaaataaatg

SEQ ID NO: 56 - DNA, 11 bp, Homo sapiens
gtgatataatc

SEQ ID NO: 57 - DNA, 11 bp, Mus sp.
gagacataaca

SEQ ID NO: 58 - DNA, 12 bp, Homo sapiens
tattctgtaaga

SEQ ID NO: 59 - DNA, 12 bp, Mus sp.
tattctgtgatg

SEQ ID NO: 60 - DNA, 12 bp, Homo sapiens
tggtgtataaga

SEQ ID NO: 61 - DNA, 12 bp, Mus sp.
cggcatataaga

SEQ ID NO: 62 - DNA, 10 bp, Homo sapiens
ttttgtatcc

SEQ ID NO: 63 - DNA, 10 bp, Mus sp.
ttttgtatct

SEQ ID NO: 64 - DNA, 12 bp, Homo sapiens
aatcatgtcatc

SEQ ID NO: 65 - DNA, 12 bp, Mus sp.
gattgtgttatc

SEQ ID NO: 66 - DNA, 12 bp, Homo sapiens
ggtctcataata

SEQ ID NO: 67 - DNA, 12 bp, Mus sp.
agtctcgcaata

SEQ ID NO: 68 - DNA, 12 bp, Homo sapiens
tgttaaataaga

SEQ ID NO: 69 - DNA, 12 bp, Homo sapiens
tttcatttaact

SEQ ID NO: 70 - DNA, 12 bp, Mus sp.
tttgatttaatt

SEQ ID NO: 71 - DNA, 12 bp, Mus sp.
tgttaaataaga

SEQ ID NO: 72 - DNA, 193 bp, Homo sapiens
acatgctccatttcaggtgtatctcatctgagacattattagagggttctctttatgtgccatgactgagtagcgttttcttccactgaagtattgcttcctctttgttgatatcacaggacagacatcagaacacttacaggatgtgtgtagtgtggcatgacagagaactttggtttcctttaatgtgact

SEQ ID NO: 73 - DNA, 187 bp, Mus sp.
acatggaccatctcgggctattttatctgagatcattggaatactctctcctcttgccatgactaatgcttttctatctctgaaatatgacttcctttttgttgattcatgtaataaacctcagagtgccttgcagggtgtatctagtgtgtcattacacagaactggaacattctctgatgtgact

SEQ ID NO: 74 - DNA, 169 bp, Homo sapiens
gctgtacagaggacaagggagagatacaaggatggccattgtaatgttgtttgtaatagcaaaagcttggaagcaacctaaatgtccgtcgatgagggagtgggtaaataaatgtgatataatcatgatacaaacagtgagtctgacaaaacatactggtatagaatgt

SEQ ID NO: 75 - DNA, 150 bp, Mus sp.
ttcagagtgcaaggcaaagataccaggatggtcatagtgatgctgtttgtaatagcaacctaaatgtccaataatggggaccgggtaaataaatgagacataacagtaacacaagcagtgagcttaattgaagagcctcatatagactgt

SEQ ID NO: 76 - DNA, 119 bp, Homo sapiens
tggccttcacatctgagaagcattgtcacagcgaatcatcctccaaacaggactgcagcagtagcttcctctttattctgtaagacatggcttgcagttttcctgaaatggagtaacct

SEQ ID NO: 77 - DNA, 106 bp, Mus sp.
tgcctctcaaatcagagaatcattggcacagccaatcctcctcccaacagggcagccatagtttcctcatttattctgtgatgcattgcttgaagaaatggaccct

SEQ ID NO: 78 - DNA, 207 bp, Homo sapiens
ttgaagcaattgtgaatgggagttcactcatgatttggctctctgtttgtctgttattggtgtataagaatgcttgtgatttttgcacgttgattttgtatcctgagactgctgaagttgcttatcagcttaaggagattttgggctgagactgtggggttttctaaatacacaatcatgtcatctgtaaacagggacaatttgact

SEQ ID NO: 79 - DNA, 183 bp, Mus sp.
ttgcaactattgtgaatggattgttttcttgatttctttctcatcggatttgttatcggcatataagaaagctttgagttttgtgtattgattttgtatctaagcatcaactctaagagttttcagtctttggtgtgtcttacatacacgattgtgttatctataaatagaaatgcttcgact

SEQ ID NO: 80 - DNA, 170 bp, Homo sapiens
tgttccttgtgccaggcaaactgccagacacatagtagaggtctcataatatttgttaaataagaaaaatgcatctttattattattttctaacacagtccatataactatctaaggcacacgttgagagtttcatttaactcaatctgagatgagaccagatttcccta

SEQ ID NO: 81 - DNA, 154 bp, Mus sp.
tgtccctggtgtctatcaaactgctgcacatacagaataagtctcgcaatatttgttaaataagagctatacactgatattatttttaacatagtcataagacagaactggtcatttgatttaattcgttctaaggtaagaccaaatttcacta